(19) United States
(12) Patent Application Publication
(10) Pub. No.: US 2025/0263448 A1
(43) Pub. Date: Aug. 21, 2025

(54) VETERINARY VACCINES AND METHODS FOR THE TREATMENT OF PASTEURELLA MULTOCIDA INFECTIONS IN FOOD PRODUCTION ANIMALS

(71) Applicant: ENGINEERED ANTIGENS INC., Calgary (CA)

(72) Inventors: Trevor MORAES, Toronto (CA); Anthony Bernard SCHRYVERS, Calgary (CA); Jamie FEGAN, Toronto (CA); Epshita ISLAM, Toronto (CA); Scott GRAY-OWEN, Oakville (CA); Rafael FRANDOLOSO, Rio Grande do Sul (BR)

(21) Appl. No.: 18/858,473

(22) PCT Filed: Apr. 20, 2023
(86) PCT No.: PCT/CA2023/050537

§ 371 Date: Oct. 21, 2024

Related U.S. Application Data
(60) Provisional application No. 63/332,966, filed on Apr. 20, 2022.

(51) Int. Cl. C07K 14/285; A61K 39/00; A61P 31/04; C12N 15/70

(52) U.S. Cl. CPC C07K 14/285; A61P 31/04; C12N 15/70; A61K 39/00; A61K 2039/552; A61K 2039/55505; A61K 2039/55561; A61K 2039/55566

(57) ABSTRACT

Disclosed are novel veterinary vaccine compositions comprising a P. multocida PmSLP protein or an immunogenically equivalent portion thereof. The vaccine compositions may be used to ameliorate, treat or prevent pathogenic infections of food production animals, such as bovine and porcine animals, caused by Pasteurella multocida. Related methods and uses are also disclosed.

### RELATED APPLICATION

This application claims the benefit of priority U.S. Provisional Application No. 63/332,966 filed Apr. 20, 2022; the entire contents of U.S. Provisional Application No. 63/332,966 are hereby incorporated by reference.

### INCORPORATION OF SEQUENCE LISTING

A computer readable form of the Sequence Listing “21806-P64683PC01_SequenceListing.xml” (148,616 bytes), was created on Apr. 19, 2023, is filed herewith by electronic submission and is incorporated by reference. herein.

### FIELD OF THE DISCLOSURE

The methods, uses and compositions disclosed herein relate to the treatment of infectious diseases. In particular, the methods, uses and compositions disclosed herein relate to veterinary vaccines to prevent, treat or ameliorate infections in food production animals caused by the infectious Gram-negative bacterium Pasteurella multocida.

### BACKGROUND OF THE DISCLOSURE

The following paragraphs are provided by way of background to the present disclosure. They are not however an admission that anything discussed therein is prior art or part of the knowledge of persons skilled in the art.

Pasteurella multocida is a Gram-negative bacterium that is able to colonize and infect a large array of different animals, including mammals and birds. The resultant diseases vary by animal species and include respiratory tract diseases in ruminants, bovine respiratory disease (BRD) and hemorrhagic septicemia (HS) in cattle and Bovinae species, porcine pneumonic pasteurellosis and porcine atrophic rhinitis (PAR) in swine, and fowl cholera in avian species.

P. multocida is a rod-shaped, non-flagellated bacterium frequently found in the oral, nasal, and respiratory tract of animals. However, disease is often associated with further organic or systemic dissemination of the bacterium and can include symptoms such as pneumonia, atrophic rhinitis, dermonecrosis, cellulitis, abscesses, meningitis, and hemorrhagic septicemia. Furthermore, P. multocida infections can be either chronic or acute (Harper M. et al., 2006, FEMS Microbiol. Letters, 265 (1), 1-10. doi: 10.1111/j. 1574-6968.2006.00442.x; Wilson, B. & Ho, M., 2013, Clinical Microbiol. Rev., 26 (3), 631. doi: 10.1128/CMR.00024-13).

Taxonomically, P. multocida can be subdivided into three subspecies: P. multocida subspecies multocida, P. multocida subspecies gallicida, and P. multocida subspecies septica (Mutters R. et al., 1985, Intern. J. of Syst. Evol. Microbiol., 35 (3), 309-322. doi.org/10.1099/00207713-35-3-309). Furthermore, P. multocida can be classified in accordance with serogroups, representing different types of extracellular capsular polysaccharides. In this respect, five serogroups (serogroup A, B, D, E, and F) are commonly distinguished (Carter, G. 1955, Rev Sci Tech, 19 (2), 626-637. doi: 10.20506/rst.19.2.1236). P. multocida strains can yet further be stratified into 16 serotypes (serotype 1-16) based on the antigenic membrane lipopolysaccharide (LPS) constituents different P. multocida strains may exhibit (Heddleston, K., 1972, Avian Dis, 16 (4), 925-936).

Bovine-associated diseases caused by P. multocida include bovine respiratory disease complex (BRD) and hemorrhagic septicemia (HS), with BRD being common in feedlots across North America and Europe, while HS is a frequent cause of disease in smallholder farmers across Asia and Africa. BRD, alternatively known as “shipping fever” in feedlot cattle, is overall considered the leading cause of cattle morbidity and mortality in feedlots and has been estimated to cause between 45-55% of all feedlot deaths (Johnson, K. & Pendell, D., 2017, Frontiers in Vet. Sci., 4 (189). doi: 10.3389/fvets.2017.00189). Furthermore, BRD has been estimated to be one of the costliest diseases in commercial North American feedlots (Griffin, D., 1997, Vet. Clin. North Am. Food Animal Practice, 13 (3), 367-377. doi: 10.1016/s0749-0720 (15) 30302-9. In this respect, BRD is commonly associated with infections of the lungs, causing pneumonia in recently weaned and feedlot cattle, nursing beef calves, housed dairy calves, and lactating dairy cows. BRD is more common among herds kept in tight quarters such as feedlots or in large herds kept on a small number of acres. BRD is additionally more typical among stressed animals and in animals with pre-existing infections, for example, pre-weaned calves and calves shortly following weaning, shipped to new locations (Wilson et al., B. K. 2017 J. Animal Sci., 95 (5), 2170-2182. doi: 10.2527/jas.2016.1006; Dubrovsky, S. et al., J Dairy Sci., 2019, 102 (8): 7320-7328. doi: 10.3168/jds.2018-15463).

Hemorrhagic septicemia (HS) is a rapidly progressing, highly fatal septicemic disease of cattle and buffaloes. HS causes significant economic losses in tropical regions of the world, especially in low and middle-income countries in Africa and Asia. HS is particularly devastating to smallholder farmers where husbandry and preventive practices are often inadequate. HS causing P. multocida can colonize the tonsils of a small proportion of healthy water buffalo and cattle (carriers) and can be shed during periods of stress, such as high temperature and humidity (Shivachandra, S. et al., 2011, Animal Health Res. Rev., 12 (1), 67-82. doi: 10.1017/S146625231100003X). Outbreaks of disease are most prevalent during the rainy season. Infection occurs by contact with infected oral or nasal secretions from either healthy carrier animals or animals with disease, or possibly by ingestion of contaminated feed or water (OIE Manual of Diagnostic Tests and Vaccines for Terrestrial Animals, 8th Edition, 2018, Chapter 3.4.10, 1125-1138). The development of disease from colonized cattle is incompletely understood but typically there is a combination of local involvement in the neck region and systemic spread which culminates in dissemination to various organs, tissue injury, cytokine storm, and toxic shock. Clinical signs can appear 1-3 days after the organism is first detected, and death can occur as rapidly as within 8-24 hours after the onset of symptoms. The economic loss due to HS has been estimated at almost $800 million USD per year in India (Singh, B. et al., 2014, Agric. Econ. Res. Rev., 27 (347-2016-17135), 271-279).

Current veterinary vaccines available for ruminant-associated P. multocida infections are limited and mainly focused on traditional bacterin formulations (killed whole bacteria) or live attenuated bacteria (streptomycin-dependent mutant strains). Both of these vaccines have unreliable efficacy profiles due to limited protection, partially due to bacterial polysaccharide capsule specificity of these vaccines, and the potential of unmatched circulating strains, but even when the capsule is matched the efficacy is incomplete and not well documented in the literature (Dabo, S. et al., 2007, Anim. Health Res. Rev., 8 (2), 129-150). Moreover, due to the limited effectiveness of existing vaccines against P. multocida and other BRD causing bacteria, prophylactic and metaphylactic use of antimicrobials is pervasive in cattle farming and is a potential important source of the development of antimicrobial resistance in pathogens (Cameron, A. & McAllister T., 2016, J. Animal Sci. and Biotechn., 7 (1), 68. doi: 10.1186/s40104-016-0127-3).

P. multocida infections in swine can cause porcine pneumonic pasteurellosis and progressive atrophic rhinitis (PAR) infections which are of considerable economic importance worldwide (Adlam C. & Rutter J, 1989, Pasteurella multocida: molecular biology, toxins and infection (Vol. 361): Springer Science & Business Media). PAR is associated with certain toxigenic strains of P. multocida, which are frequently serogroup D and, to a lesser extent, serogroup A (Eamens G. et al., 1988, Aust. Vet. J, 65 (4), 120-123. doi: 10.1111/j.1751-0813.1988.tb14430.x; Foged N. et al., 1989, Vet. Rec., 125, 7-11; Fussing, V. et al., Vet. Microbiol, 65 (1), 61-74. doi: 10.1016/s0378-1135 (98) 00288-0; and Sakano T. et al., 1992, J. Vet. Med. Sci., 54 (3), 403-407. doi: 10.1292/jvms.54.403), while pneumonic pasteurellosis is generally caused by non-toxigenic strains but also encompasses serogroups A and D (Djordjevic, S. et al., 1998, J. Med. Microbiol., 47 (8), 679-688. doi.org/10.1099/00222615-47-8-679; Pijoan, C. et al., 1983, J. Clin. microbiol., 17 (6), 1074-1076; and Zhao, G. et al., 1992, Infect. Immun., 60 (4), 1401-1405). P. multocida-associated swine infections occur globally, including in North America, Europe, and Asia (VanderWaal D. & Deen J., 2018, PNAS 115 (45), 11495. doi: 10.1073/pnas. 1806068115). As is the case in cattle rearing, P. multocida-associated infections are of significant economic burden and management of these infections leads to increased antibiotic usage. Swine vaccines against P. multocida are generally either bacterins of toxigenic and non-toxigenic strains or toxoid-based vaccines (OIE Manual of Diagnostic Tests and Vaccines for Terrestrial Animals, 8^{th} Edition, 2018, Chapter 3.8.2, 1540-1550). Vaccines against pneumonic pasteurellosis in swine do not appear to be wildly available.

P. multocida is also the causative agent of fowl cholera and is associated with a wide variety of infections. Acute infections can progress rapidly, and often sudden death of birds within flocks is the only sign that infection is occurring within a facility, however other symptoms can include discharge from the mouth and nose, cyanosis, general depression, and diarrhea (Christensen, J. & Bisgaard, M. 2000, Rev. Sci. Tech., 19 (2), 626-637. doi: 10.20506/rst. 19.2.1236). Fowl cholera can effect a variety of avian species, with chicken, turkey, duck, and quail being of the most economic importance (Glisson, J. 1998, Poultry Science, 77 (8), 1139-1142. doi.org/10.1093/ps/77.8.1139). Fowl cholera is mostly caused by serogroup A strains of P. multocida, however serogroups F and D have also been reported (Dziva, F. et al., 2008, Vet. Microbiol., 128 (1-2), 1-22. doi: 10.1016/j.vetmic.2007.10.018). Available inactivated bacterins and live attenuated vaccines. Inactivated vaccines elicit a serotype specific response, thus offering limited protection. On the other hand, live inactivated vaccines do appear to elicit a broad, cross-serotype response. However live attenuated vaccines have the potential to induce chronic fowl cholera in chickens and turkeys (Glisson, J. 1998, Poultry Science, 77 (8), 1139-1142. doi.org/10.1093/ps/77.8.1139).

In light of the foregoing, there exists a need in the art for improved methods and compositions to treat P. multocida infections. In particular, there is a need in the art for improved vaccines to prevent disease caused by Pasteurella multocida infections in food production animals, including but not limited to ruminant such as bovine, porcine, and avian food production animals.

### SUMMARY OF THE DISCLOSURE

The following paragraphs are intended to introduce the reader to the more detailed description, not to define or limit the claimed subject matter of the present disclosure.

In one aspect, the present disclosure relates to vaccine formulations.

In another aspect, the present disclosure relates to vaccine formulations to prevent or ameliorate diseases in food production animals that are caused by the bacterial pathogen Pasteurella multocida (P. multocida).

The inventors have discovered that the veterinary vaccine formulations of the present disclosure can provide protection to food production animals against infection by multiple P. multocida strains, including in a cross-protective manner, using a single immunogenic active agent. The immunogenic agents discovered to be effective when administered to a food production animal in a veterinary vaccine formulation are, in particular, proteins selected from a class of P. multocida proteins, known as PmSLP proteins.

Accordingly, the present disclosure provides, in accordance with the teachings herein, in at least one embodiment, a veterinary vaccine formulation for the prevention or amelioration of P. multocida infection in a food production animal susceptible to P. multocida infection, the vaccine formulation comprising an effective amount of at least one PmSLP protein, or an immunogenically equivalent portion thereof.

In at least one embodiment, in an aspect, the vaccine formulation can comprise at least one P. multocida PmSLP protein, or an immunogenically equivalent portion thereof selected from the same phylogenetic cluster as a PmSLP protein present in the infecting P. multocida strain.

In at least one embodiment, in an aspect, the vaccine formulation can comprise a P. multocida PmSLP protein, or an immunogenically equivalent portion thereof, selected from the group of phylogenetic clusters consisting of PmSLP-1, PmSLP-2, PmSLP-3, PmSLP-4.1, and PmSLP-4.2, wherein the selected P. multocida PmSLP protein, or an immunogenically equivalent portion thereof, belongs to the same phylogenetic cluster as a PmSLP protein present in the infecting P. multocida strain.

In at least one embodiment, in an aspect, the food production animal can be a ruminant animal susceptible to infection by a P. multocida strain causing a respiratory tract disease, and the vaccine formulation comprises a P. multocida PmSLP protein from a respiratory tract disease causing P. multocida strain, or an immunogenically equivalent portion thereof, wherein the PmSLP protein is selected from the group of phylogenetic clusters consisting of PmSLP-1, PmSLP-2, PmSLP-3, and PmSLP-4.2, wherein the selected P. multocida PmSLP protein, or an immunogenically equivalent portion thereof, belongs to the same phylogenetic cluster as a PmSLP protein present in the infecting P. multocida strain.

In at least one embodiment, in an aspect, the food production animal can be a bovine animal susceptible to infection by a P. multocida strain causing BRD, and the vaccine formulation comprises a P. multocida PmSLP protein from a BRD causing P. multocida strain, or an immunogenically equivalent portion thereof, wherein the PmSLP protein is selected from the group of phylogenetic clusters consisting of PmSLP-1, PmSLP-2, and PmSLP-4.2, wherein the selected P. multocida PmSLP protein, or an immunogenically equivalent portion thereof, belongs to the same phylogenetic cluster as a PmSLP protein present in the infecting P. multocida strain.

In at least one embodiment, in an aspect, the food production animal can be a bovine animal susceptible to infection by a P. multocida strain causing HS, and the vaccine formulation comprises a P. multocida PmSLP protein from an HS causing P. multocida strain, or an immunogenically equivalent portion thereof, wherein the PmSLP protein is selected from the phylogenetic cluster PmSLP-3 wherein the selected P. multocida PmSLP protein, or an immunogenically equivalent portion thereof, belongs to the same phylogenetic cluster as a PmSLP protein present in the infecting P. multocida strain.

In at least one embodiment, in an aspect, the food production animal can be a porcine animal susceptible to infection by a P. multocida strain causing porcine atrophic rhinitis (PAR), and the vaccine formulation comprises a P. multocida PmSLP protein from a PAR causing P. multocida strain, or an immunogenically equivalent portion thereof, wherein the PmSLP protein is selected from the group of phylogenetic clusters consisting of PmSLP-2, PmSLP-4.1, and PmSLP-4.2, wherein the selected P. multocida PmSLP protein, or an immunogenically equivalent portion thereof, belongs to the same phylogenetic cluster as a PmSLP protein present in the infecting P. multocida strain.

In at least one embodiment, in an aspect, the food production animal can be a porcine animal susceptible to infection by a P. multocida strain causing pneumonic pasteurellosis, and the vaccine formulation comprises a P. multocida PmSLP protein from a pneumonic pasteurellosis causing P. multocida strain, or an immunogenically equivalent portion thereof, wherein the PmSLP protein is selected from the group of phylogenetic clusters consisting of PmSLP-2, PmSLP-4.1, and PmSLP-4.2, wherein the selected P. multocida PmSLP protein, or an immunogenically equivalent portion thereof, belongs to the same phylogenetic cluster as a PmSLP protein present in the infecting P. multocida strain.

In at least one embodiment, in an aspect, the food production animal can be an avian animal susceptible to infection by a P. multocida strain causing fowl cholera, and the vaccine formulation comprises a P. multocida PmSLP protein from a fowl cholera causing P. multocida strain, or an immunogenically equivalent portion thereof, wherein the PmSLP protein is selected from the group of phylogenetic clusters consisting of PmSLP-3 and PmSLP-4.2, wherein the selected P. multocida PmSLP protein, or an immunogenically equivalent portion thereof, belongs to the same phylogenetic cluster as a PmSLP protein present in the infecting P. multocida strain.

In at least one embodiment, in an aspect, the vaccine formulation can comprise a P. multocida PmSLP protein, or an immunogenically equivalent portion thereof, selected from the group of phylogenetic clusters consisting of PmSLP-1, PmSLP-2, PmSLP-3, PmSLP-4.1, and PmSLP-4.2, wherein the selected P. multocida PmSLP protein, or an immunogenically equivalent portion thereof, belongs to the same phylogenetic cluster as a PmSLP protein present in the infecting P. multocida strain, and wherein the selected P. multocida PmSLP protein, or an immunogenically equivalent portion thereof, further is of a P. multocida strain belonging to a serogroup selected from the group consisting of serogroup A, B, D, E, and F, wherein the serogroup is the same as the serogroup of the infecting P. multocida strain.

In at least one embodiment, in an aspect, the at least one PmSLP protein, or immunologically equivalent portion thereof, can be a protein expressed by a nucleic acid sequence selected from the group of nucleic acid sequences consisting of:
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence having at least 70% identity with any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a chimeric nucleic acid obtained by a fusion between at least two nucleic acid sequences of (a), (b), (c), and (d), or a portion thereof;
(f) a nucleic acid sequence that is complementary to any one of the nucleic acid sequences of (a);
(g) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(h) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereto; and
(i) a nucleic acid sequence that hybridizes under stringent conditions to any one of the nucleic acid sequences set forth in (a), (b), (c), (d), (e), (f), (g), or (h).

In at least one embodiment, in an aspect, the at least one P. multocida PmSLP protein, or an immunogenically equivalent portion thereof, can comprise any one of the amino acid sequences set forth in SEQ. ID NO: 2, SEQ. ID NO: 4, SEQ. ID NO: 6, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 65, SEQ. ID NO: 67, SEQ. ID NO: 69, SEQ. ID NO: 71, SEQ. ID NO: 73, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, or SEQ. ID NO: 91, or an immunogenically equivalent portion thereof, or is a chimeric protein obtained by fusion of any one of said sequences or portion thereof.

In at least one embodiment, in an aspect, the food production animal can be a ruminant species, and the P. multocida infection causes respiratory tract disease.

In at least one embodiment, in an aspect, the food production animal can be a bovine species, and the P. multocida infection causes bovine respiratory disease (BRD) or hemorrhagic septicemia (HS).

In at least one embodiment, in an aspect, the food production animal can be a porcine species and the P. multocida infection causes porcine pneumonic pasteurellosis or porcine atrophic rhinitis (PAR).

In at least one embodiment, in an aspect, the food production animal can be an avian species, and the P. multocida infection causes fowl cholera.

In at least one embodiment, in an aspect, the food production animal can be a ruminant species, wherein the P. multocida infection causes a respiratory tract disease, and wherein the PmSLP protein comprises SEQ. ID NO: 2, SEQ. ID NO: 4, SEQ. ID NO: 6, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 65, SEQ. ID NO: 67, SEQ. ID NO: 69, SEQ. ID NO: 71, SEQ. ID NO: 73, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, or SEQ. ID NO: 91, or an immunogenically equivalent portion thereof, to prevent or ameliorate a P. multocida infection causing the respiratory tract disease.

In at least one embodiment, in an aspect, the food production animal can be a bovine species, wherein the P. multocida infection causes BRD, and wherein the PmSLP protein comprises SEQ. ID NO: 2, SEQ. ID NO: 4, SEQ. ID NO: 10, SEQ. ID NO: 12, SEQ. ID NO: 14 SEQ. ID NO: 16, SEQ. ID NO: 18, SEQ. ID. NO: 22, SEQ. ID. NO: 26, SEQ. ID. NO: 28, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 65, SEQ. ID NO: 67, SEQ. ID NO: 69, SEQ. ID NO: 71, SEQ. ID NO: 73, SEQ. ID NO: 77, or SEQ. ID NO: 91, or an immunogenically equivalent portion thereof, to prevent or ameliorate a P. multocida infection causing BRD.

In at least one embodiment, in an aspect, the food production animal can be a bovine species, wherein the P. multocida infection causes HS, and wherein the PmSLP protein comprises SEQ. ID NO: 6, SEQ. ID NO: 20, SEQ. ID NO: 24, SEQ. ID NO: 30, SEQ. ID NO: 32, SEQ. ID NO: 34, SEQ. ID NO: 36, SEQ. ID NO: 53, SEQ. ID NO: 79, SEQ. ID NO: 81, SEQ. ID NO: 83, SEQ. ID NO: 85, SEQ. ID NO: 87, or SEQ. ID NO: 89, or an immunogenically equivalent portion thereof, to prevent or ameliorate a P. multocida infection causing HS.

In at least one embodiment, in an aspect, the food production animal can be a porcine species, and the P. multocida infection causes pneumonic pasteurellosis, and wherein the PmSLP protein comprises SEQ. ID NO: 4, SEQ. ID NO: 8, SEQ. ID NO: 10, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 75, SEQ. ID NO: 77, or SEQ. ID NO: 91, or an immunogenically equivalent portion thereof, to prevent or ameliorate a P. multocida infection causing pneumonic pasteurellosis.

In at least one embodiment, in an aspect, the food production animal can be a porcine species, wherein the P. multocida infection causes PAR, and wherein the PmSLP protein comprises SEQ. ID NO: 4, SEQ. ID NO: 8, SEQ. ID NO: 10, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 75, SEQ. ID NO: 77, or SEQ. ID NO: 91, or an immunogenically equivalent portion thereof, to prevent or ameliorate a P. multocida infection causing PAR.

In at least one embodiment, in an aspect, the food production animal can be an avian species, wherein the P. multocida infection causes fowl cholera, and wherein the PmSLP protein comprises SEQ. ID NO: 6, SEQ. ID NO: 10, SEQ. ID NO: 20, SEQ. ID NO: 24, SEQ. ID NO: 30, SEQ. ID NO: 32, SEQ. ID NO: 34, SEQ. ID NO: 36, SEQ. ID NO: 40, SEQ. ID NO: 53, SEQ. ID NO: 55, SEQ. ID NO: 61, SEQ. ID NO: 77, SEQ. ID NO: 79, SEQ. ID NO: 81, SEQ. ID NO: 83, SEQ. ID NO: 85, SEQ. ID NO: 87, or SEQ. ID NO: 89, or an immunogenically equivalent portion thereof, to prevent or ameliorate a P. multocida infection causing pneumonic pasteurellosis.

In at least one embodiment, in an aspect, the PmSLP protein, or immunogenically equivalent portion thereof, can be recombinantly produced in a microbial host organism.

In at least one embodiment, in an aspect, the veterinary vaccine formulation can be a cross-protective vaccine formulation comprising a PmSLP protein, or immunogenically equivalent portion thereof, obtained from a first P. multocida strain, and the vaccine formulation is a formulation for the administration to the food production animal to prevent or ameliorate an infection caused by another P. multocida strain.

In at least one embodiment, in an aspect, the vaccine formulation can be substantially free of other P. multocida constituents.

In at least one embodiment, in an aspect, the PmSLP protein, or immunologically equivalent portion thereof, can be a recombinantly produced protein, wherein the vaccine formulation is substantially free of host cell constituents.

In at least one embodiment, in an aspect, wherein the vaccine formulation furthers can comprise a veterinary pharmaceutically acceptable adjuvant.

In at least one embodiment, in an aspect, wherein the vaccine formulation can further comprise a veterinary pharmaceutically acceptable excipient, carrier, or diluent.

In at least one embodiment, in an aspect, wherein the vaccine formulation can comprise from about 0.001% to about 20% by weight of the PmSLP protein or the immunogenically equivalent portion thereof, and a veterinary pharmaceutically acceptable adjuvant constituting from about 0.1% to about 60% by weight or volume of the vaccine formulation.

In at least one embodiment, in an aspect, the vaccine formulation can comprise a second P. multocida PmSLP protein, or immunologically equivalent portion thereof.

In at least one embodiment, in an aspect, the vaccine formulation can comprise a fusion polypeptide comprising the first and second P. multocida PmSLP protein, or immunologically equivalent portion thereof.

In at least one embodiment, in an aspect, the second P. multocida PmSLP protein, or immunologically equivalent portion thereof, can belong to the same or a different phylogenetic cluster as the first P. multocida PmSLP protein, or immunologically equivalent portion thereof.

In at least one embodiment, in an aspect, the fusion polypeptide can comprise a fusion polypeptide selected from the group consisting of a (i) PmSLP protein, or immunologically equivalent portion thereof, belonging to phylogenetic cluster 1 and a PmSLP protein, or immunologically equivalent portion thereof, belonging to phylogenetic cluster 3; (ii) PmSLP protein, or immunologically equivalent portion thereof, belonging to phylogenetic cluster 1 and a PmSLP protein, or immunologically equivalent portion thereof, belonging to phylogenetic cluster 2; (iii) a PmSLP protein, or immunologically equivalent portion thereof, belonging to phylogenetic cluster 1 and a PmSLP protein, or immunologically equivalent portion thereof, belonging to phylogenetic cluster 4.1; and (iv) a PmSLP protein, or immunologically equivalent portion thereof, belonging to phylogenetic cluster 1 and a PmSLP protein, or immunologically equivalent portion thereof, belonging to phylogenetic cluster 4.2.

In at least one embodiment, in an aspect, the second P. multocida PmSLP protein, or immunologically equivalent portion thereof, can be obtained from a P. multocida strain belonging to the same or a different serogroup as the P. multocida strain of the first P. multocida PmSLP protein, or immunologically equivalent portion thereof.

In another aspect, the present disclosure provides, in accordance with the teachings herein, in at least one embodiment, a use of a veterinary vaccine formulation for the prevention, treatment or amelioration of P. multocida infection in a food production animal susceptible to P. multocida infection, the vaccine formulation comprising an effective amount of at least one PmSLP protein, or an immunogenically equivalent portion thereof. In another embodiment, the present disclosure provides a veterinary vaccine formulation for use in the prevention, treatment or amelioration of P. multocida infection in a food production animal susceptible to P. multocida infection, the vaccine formulation comprising an effective amount of at least one PmSLP protein, or an immunogenically equivalent portion thereof.

In at least one embodiment, in an aspect, the vaccine formulation can cause an improved trending towards normal of one or more clinical parameters selected from the group consisting of (i) rectal temperature, (ii) animal demeanor, (iii) nasal discharge pattern, (iv) coughing pattern, (v) respirational pattern, and (vi) overall clinical health, relative to an animal production animal not having been administered the veterinary vaccine formulation.

In at least one embodiment, in an aspect, the vaccine formulation can be capable of eliciting an immune response in the food production animal, wherein anti-PmSLP antibodies are detectable in the blood serum of the food production animal at least in the period starting 7 days and ending 52 weeks from the date of use of the vaccine formulation.

In another aspect, the present disclosure provides, in accordance with the teachings herein, in at least one embodiment, a method for prevention, treatment or amelioration of P. multocida infection in a food production animal susceptible to Pasteurella multocida (P. multocida) infection, the method comprising administering to the food production animal a veterinary vaccine formulation comprising a P. multocida PmSLP protein, or an immunogenically equivalent portion thereof, wherein the vaccine formulation is administered in an effective amount to prevent, treat or ameliorate the P. multocida infection.

In at least one embodiment, in an aspect, the amelioration of the P. multocida infection can comprise the reduction of clinical signs of any disease caused by the P. multocida infection.

In at least one embodiment, in an aspect, the clinical signs dan be selected from the group consisting of: (i) rectal temperature, (ii) animal demeanor, (iii) nasal discharge pattern, (iv) coughing pattern, (v) respirational pattern, and (vi) overall clinical health, relative to an animal production animal not having been administered the veterinary vaccine formulation.

In at least one embodiment, in an aspect, the food production animal susceptible to Pasteurella multocida infection can be selected from the group consisting of ruminant, porcine, and avian species.

In at least one embodiment, in an aspect, the disease caused by Pasteurella multocida infection can be selected from the group consisting of respiratory tract disease, bovine respiratory disease (BRD), hemorrhagic septicemia (HS), porcine atrophic rhinitis (PAR), and fowl cholera.

In at least one embodiment, in an aspect, the food production animal can be a ruminant susceptible to P. multocida infection, and wherein the veterinary vaccine formulation administered in an effective amount to the ruminant comprises at least one PmSLP protein from a P. multocida strain causing respiratory tract disease, or an immunogenically equivalent portion thereof.

In at least one embodiment, in an aspect, the food production animal susceptible to P. multocida infection can be a bovine species, and wherein the veterinary vaccine formulation administered in an effective amount to the bovine species comprises at least one PmSLP protein from a P. multocida strain causing BRD, or an immunogenically equivalent portion thereof.

In at least one embodiment, in an aspect, the food production animal susceptible to P. multocida infection can be a ruminant, and wherein the veterinary vaccine formulation administered in an effective amount to the ruminant comprises at least one PmSLP protein from a P. multocida strain causing HS, or an immunogenically equivalent portion thereof.

In at least one embodiment, in an aspect, the food production animal susceptible to infection by a P. multocida strain can be a porcine animal, and wherein the veterinary vaccine formulation administered in an effective amount to the porcine animal comprises at least one PmSLP protein from a P. multocida causing porcine atrophic rhinitis (PAR), or an immunogenically equivalent portion thereof.

In at least one embodiment, in an aspect, the food production animal susceptible to infection by a P. multocida strain can be an avian animal, and wherein the veterinary vaccine formulation administered in an effective amount to the avian animal comprises at least one PmSLP protein from a fowl cholera causing P. multocida strain, or an immunogenically equivalent portion thereof.

In at least one embodiment, in an aspect, the vaccine formulation administered in an effective amount to the food production animal can comprise at least one P. multocida PmSLP protein, or an immunogenically equivalent portion thereof, selected from the group of phylogenetic clusters consisting of PmSLP-1, PmSLP-2, PmSLP-3, PmSLP-4.1, and PmSLP-4.2.

In at least one embodiment, in an aspect, the veterinary vaccine formulation can comprise at least one P. multocida PmSLP protein, or an immunogenically equivalent portion thereof, which confers to the food production animal susceptible to infection by P. multocida a homologous protection against a P. multocida strain, possessing a PmSLP protein from the same phylogenetic cluster.

In at least one embodiment, in an aspect, the veterinary vaccine formulation can comprise at least one P. multocida PmSLP protein, or an immunogenically equivalent portion thereof, which confers to the food animal susceptible to infection by P. multocida heterologous protection against a P. multocida strain, possessing a PmSLP protein from a different phylogenetic cluster.

In at least one embodiment, in an aspect, the vaccine formulation can comprise at least one P. multocida PmSLP protein, or an immunogenically equivalent portion thereof, selected from a protein encoded by a nucleic acid sequence selected from the group of nucleic acid sequences consisting of:
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence having at least 70% identity with any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a chimeric nucleic acid obtained by a fusion between at least two nucleic acid sequences of (a), (b), (c), and (d) or portion thereof;
(f) a nucleic acid sequence that is complementary to any one of the nucleic acid sequences of (a);
(g) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(h) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereto; and
(i) a nucleic acid sequence that hybridizes under stringent conditions to any one of the nucleic acid sequences set forth in (a), (b), (c), (d), (e), (f), (g), or (h).

In at least one embodiment, in an aspect, the vaccine formulation can comprise at least one P. multocida PmSLP protein, or an immunogenically equivalent portion thereof, having any one of amino acid sequences set forth in SEQ. ID NO: 2, SEQ. ID NO: 4, SEQ. ID NO: 6, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 65, SEQ. ID NO: 67, SEQ. ID NO: 69, SEQ. ID NO: 71, SEQ. ID NO: 73, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, or SEQ. ID NO: 91, or an immunogenically equivalent portion thereof, or a chimeric protein obtained by a fusion between any one of said sequences or portion thereof.

In at least one embodiment, in an aspect, the PmSLP protein, or immunogenically equivalent portion thereof can be recombinantly produced in a microbial host organism.

In at least one embodiment, in an aspect, the PmSLP protein, or immunologically equivalent portion thereof, can be substantially free of other P. multocida constituents.

In at least one embodiment, in an aspect, the PmSLP protein, or immunologically equivalent portion thereof, can be a recombinantly produced protein, wherein the PmSLP protein, or immunologically equivalent portion thereof, is substantially free of host cell constituents.

In at least one embodiment, in an aspect, the vaccine formulation further can comprise a veterinary pharmaceutically acceptable adjuvant.

In at least one embodiment, in an aspect, the vaccine formulation can comprise a veterinary pharmaceutically acceptable excipient, carrier, or diluent.

In another aspect, the present disclosure provides, in accordance with the teachings herein, in at least one embodiment, a method for preparing a veterinary vaccine formulation for the prevention or amelioration of P. multocida infection in a food production animal susceptible to P. multocida infection, the method comprising:
(i) diagnosing a P. multocida infection in a food production animal;
(ii) identifying the phylogenetic cluster to a which a PmSLP protein present in the infecting P. multocida belongs, the phylogenetic cluster being selected from PmSLP-1, PmSLP-2, PmSLP-3, PmSLP-4.1 or PmSLP-4.2; and
(iii) preparing a vaccine formulation comprising a P. multocida PmSLP protein, or immunogenically equivalent portion thereof, which belongs to the identified phylogenetic cluster together with a veterinary pharmaceutically acceptable adjuvant to form a veterinary vaccine formulation comprising an effective amount of the P. multocida PmSLP protein or the immunogenically equivalent portion thereof to treat a food production animal susceptible to P. multocida infection.

In at least one embodiment, in an aspect, the method additionally can comprise identifying the serogroup of the infecting P. multocida strain, the serogroup being selected from the group consisting of serogroup A, B, D, E, and F, and the vaccine being prepared using a P. multocida PmSLP protein, or an immunogenically equivalent portion thereof, from the same or another P. multocida strain belonging to the selected serogroup.

In another aspect, the present disclosure provides, in accordance with the teachings herein, in at least one embodiment, a method for preparing a veterinary vaccine formulation comprising a P. multocida PmSLP protein, or an immunogenically equivalent portion thereof, the method comprising:
(a) providing a chimeric nucleic acid sequence comprising as operably linked components:
   (i) a nucleic acid sequence encoding a P. multocida PmSLP protein, or an immunogenically equivalent portion thereof; and
   (ii) one or more nucleic acid sequences capable of controlling expression of the nucleic acid sequence encoding the PmSLP protein, or an immunogenically equivalent portion thereof in a host cell;
(b) introducing the chimeric nucleic acid sequence into a host cell;
(c) growing the host cell to produce the P. multocida PmSLP protein or the immunogenically equivalent portion thereof; and
(d) recovering the P. multocida PmSLP protein or the immunogenically equivalent portion thereof; and
(e) formulating the P. multocida PmSLP protein or the immunogenically equivalent portion thereof together with a veterinary pharmaceutically acceptable adjuvant to form a veterinary vaccine formulation comprising an effective amount of the P. multocida PmSLP protein or the immunogenically equivalent portion thereof to treat a food production animal susceptible to P. multocida infection.

In at least one embodiment, in an aspect, the nucleic acid sequence can be a nucleic acid sequence selected from the group of nucleic acid sequences consisting of:
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence having at least 70% identity with any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a chimeric nucleic acid obtained by a fusion between at least two nucleic acid sequences of (a), (b), (c), and (d), or a portion thereof;
(f) a nucleic acid sequence that is complementary to any one of the nucleic acid sequences of (a);
(g) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(h) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereto; and
(i) a nucleic acid sequence that hybridizes under stringent conditions to any one of the nucleic acid sequences set forth in (a), (b), (c), (d), (e), (f), (g), or (h).

In another aspect, the present disclosure provides, in accordance with the teachings herein, in at least one embodiment, an expression vector comprising:
(a) a nucleic acid sequence encoding a P. multocida PmSLP protein, or an immunogenically equivalent portion thereof; and
(b) a nucleic acid sequence capable of controlling expression of the nucleic acid sequence encoding the P. multocida PmSLP protein, or an immunogenically equivalent portion thereof in a host cell.

In at least one embodiment, in an aspect, the nucleic acid sequence can be a nucleic acid sequence selected from the group of nucleic acid sequences consisting of:
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence having at least 70% identity with any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a chimeric nucleic acid obtained by a fusion between any nucleic acid sequence of (a), (b), (c), and (d) or portion thereof;
(f) a nucleic acid sequence that is complementary to any one of the nucleic acid sequences of (a);
(g) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(h) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereto; and
(i) a nucleic acid sequence that hybridizes under stringent conditions to any one of the nucleic acid sequences set forth in (a), (b), (c), (d), (e), (f), (g), or (h).

In another aspect, the present disclosure provides, in accordance with the teachings herein, in at least one embodiment, a host cell comprising a chimeric nucleic acid comprising:
(a) a nucleic acid sequence encoding a P. multocida PmSLP protein, or an immunogenically equivalent portion thereof; and
(b) a nucleic acid sequence capable of controlling expression of the nucleic acid sequence encoding the P. multocida PmSLP protein, or an immunogenically equivalent portion thereof in a host cell.

In at least one embodiment, in an aspect, the nucleic acid sequence can be a nucleic acid sequence selected from the nucleic acid sequences consisting of:
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence having at least 70% identity with any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a chimeric nucleic acid obtained by a fusion between any nucleic acid sequence of (a), (b), (c), and (d) or portion thereof;
(f) a nucleic acid sequence that is complementary to any one of the nucleic acid sequences of (a);
(g) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(h) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereto; and
(i) a nucleic acid sequence that hybridizes under stringent conditions to any one of the nucleic acid sequences set forth in (a), (b), (c), (d), (e), (f), (g), or (h).

In another aspect, the present disclosure provides, in accordance with the teachings herein, in at least one embodiment, a use of a P. multocida PmSLP protein, or an immunogenically equivalent portion thereof, to prepare a veterinary vaccine formulation comprising the P protein or an immunogenically equivalent portion thereof together with a veterinary pharmaceutically acceptable adjuvant.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIGS. 1A, 1B, 1C, and 1D depict certain aspects of an example production and purification workflow for recombinant PmSLP proteins and example analytical data and results obtained during different steps in the performance of an example process for the purification of PmSLP-3. FIG. 1A is a flowchart showing a schematic workflow for the purification of recombinant PmSLP. FIG. 1B is a photographic image of a sodium dodecyl sulfate polyacrylamide gelelectrophoresis (SDS-PAGE) gel in which sample material collected at different stages (1-6) of nickel nitrilotriacetic acid (NTA) purification of his-tagged PmSLP-3 (SEQ. ID NO: 12) from E. coli lysate are shown. FIG. 1C is a S75 gel filtration chromatogram of size exclusion chromatography after removal of the poly-histidine tag of PmSLP-3 (SEQ. ID NO: 14). FIG. 1D is a photographic image of an SDS-PAGE gel of purified PmSLP-3 after the performance of a polishing step with the MonoQ. It is noted that PmSLP-3 migrates in the gel at approximately 35 kDa after tag removal.

FIG. 2 is a phylogenetic tree of PmSLP protein sequences collected from a certain local collection of PmSLP sequences combined with publicly available sequences from online databases, and certain information related to the phylogenetic tree. Annotations regarding host species, confirmed disease status, geographical region, and serotype, for each PmSLP variant, are based on information available in the National Center for Biotechnology Information (NCBI) BioSample database, literature searches and genomic sequence analysis. Black circles on the phylogenetic branches of the tree indicate sequences originating from bovine species; small light grey shaded dots represent other hosts. Further depicted, peripheral to the phylogenetic tree, are four ring structures, representing: (1) host (species), (2) disease, (3) (geographical) region, and (4) capsule. Each ring structure is constituted of multiple rectangular pieces, each corresponding with a single PmSLP variant of the phylogenetic tree. Thus, moving directly outwardly from a specific selected rectangular piece on the inner most ring structure, representing a specific PmSLP variant sequence, are: (1) host species (the selected rectangular piece of the inner ring structure, for example, rectangular piece (a)); (2) disease (the rectangular piece moving directly outward and immediately adjacent to the piece selected under (1), for example, rectangular piece (b)); (3) geographic region (the rectangular piece moving further directly outward and immediately adjacent to the piece identified under (2), for example, rectangular piece (c)); and (4) capsule (the rectangular piece moving further directly outward and immediately adjacent to the piece identified under (3), for example, rectangular piece (d)). Grey scale indicators for the rectangular pieces are as follows, in the innermost ring structure, representing host species: black, bovine species (including rectangular piece (a)); hatched, porcine species; intermediate grey shade, avian species; light grey shade, other species; and white, missing. For the ring structure immediately peripheral relative to the most centrally positioned ring, representing disease, grey scale indicators for the rectangular pieces are as follows: black, bovine hemorrhagic septicemia (HS); intermediate grey shade, bovine respiratory disease (BRD) (including rectangular piece (b)); and white, disease status unconfirmed/unknown. For the ring structure immediately central relative to the most peripherally positioned ring, representing the geographical region from which the samples were obtained, grey scale indicators for the rectangular pieces are as follows: black, North America; intermediate grey shade, Asia; light grey shade, Europe (including rectangular piece (c)); and white, unknown region. For the most peripherally positioned ring structure, indicating the capsular serogroup of P. multocida (where known), grey scale indicators are as follows: black, serogroup A (including rectangular piece (d)); hatched pattern, serogroup B; intermediate grey shade, serogroup D; light grey shade, serogroup F; and white, untypable. Thus, the example selected PmSLP sequence corresponding with example rectangular pieces (a), (b), (c), (d) corresponds with a bovine PmSLP isolated from a P. multocida strain causing BRD in Europe, the P. multocida strain having serogroup A. It is noted that the phylogenetic tree of PmSLP variants can be seen to separate into five phylogenetic clusters (PmSLP-1, PmSLP-2, PmSLP-3, and PmSLP-4.1, and PmSLP-4.2).

FIG. 3 is a graph depicting results obtained in the performance of a certain experiment, notably an experiment to evaluate antibody responses in mice having been administered a PmSLP vaccine formulation. In the graph endpoint IgG titre is plotted as a function of time. Serum IgG titre from mice having received 2 doses of vaccine was measured in serum samples collected over a 26-week period. Data points in the graph represent mean, error bars depict standard error. The dotted line at the 8,000 endpoint IgG titre signifies the limit of detection, which is the lowest serum dilution assayed. The IgG titre remained stable for the entire duration of the experiment and none of the groups showed any significant signs of waning. The experimental results demonstrate that long-lived antibody responses can be elicited using PmSLP-1 (SEQ. ID NO: 14) containing vaccine formulations.

FIGS. 4A, 4B, 4C, 4D, and 4E are further graphs depicting results obtained in the performance of certain experiments, notably an experiment to evaluate immune responses in mice having been administered a PmSLP vaccine formulation. In this example experiment, the efficacy of a PmSLP-1 (SEQ. ID NO: 14) vaccine formulation was evaluated against a bovine respiratory disease (BRD) isolate of P. multocida strain H246 that harbours a pmSLP gene from the same phylogenetic cluster. The graph shown in FIG. 4A depicts the percentage survival as a function of time, notably the percentage survival of mice after infection with P. multocida BRD strain H246 (the confirmed sequence of the PmSLP in this strain is defined in SEQ. ID NO: 22). PmSLP-1 vaccinated mice were 100% protected. The graph shown in FIG. 4B depicts clinical score as a function of time, notably the clinical score of mice immunized with PmSLP-1 vaccine separated by individual animal. The graph shown in FIG. 4C depicts clinical score as a function of time, notably the clinical score of mice immunized with adjuvant only separated by individual animal. The graph shown in FIG. 4D shows clinical score as a function of time, notably the mean clinical score of mice from both groups; error bars depict standard error. For FIGS. 4B, 4C, and 4D, a clinical score cut off of 10 is considered the humane endpoint at which point animals are euthanized. The graph shown in FIG. 4E shows bacterial recovery as a function of time, notably bacterial recovery from tail vein bleeds during the infection separated by individual animal. The experimental results demonstrate PmSLP vaccines can be efficacious against relevant bovine P. multocida disease isolates in an acute mouse infection model.

FIGS. 5A and 5B are further graphs depicting results obtained in the performance of certain experiments, notably an experiment to evaluate the immune response in mice having been administered a PmSLP vaccine formulation. Serum samples were obtained from mice immunized with two doses of either PmSLP-1 (SEQ. ID NO: 14) vaccine or adjuvant prior to being challenged. The graph shown in FIG. 5A depicts α-PmSLP-1 IgG titre against the purified antigen as measured by protein ELISA. The graph shown in FIG. 5B depicts α-PmSLP-1 IgG titre against whole bacteria as measured by heat inactivated whole cell ELISA. P. multocida BRD strain H246 (containing a PmSLP defined in SEQ. ID NO: 22, which harbours a pmSLP gene from the same phylogenetic cluster as the vaccine antigen was used. For both FIG. 5A and FIG. 5B, individual points depict individual animals. Bars depict mean, error bars depict standard error. The experimental results demonstrate that α-PmSLP-1 antibodies elicited in vaccinated mice can bind purified antigen as well as the antigen on the bacterial surface.

FIGS. 6A, 6B, 6C, 6D, and 6E are further graphs depicting results obtained in the performance of certain experiments, notably an experiment to evaluate the immune response in mice having been administered a PmSLP vaccine formulation. In this example, the efficacy of a PmSLP-3 (SEQ. ID NO: 20) vaccine was evaluated against a porcine disease isolate that harbours a pmSLP gene from the same phylogenetic cluster. The graph shown in FIG. 6A depicts percentage survival as a function of time, notably the percentage survival of mice after infection with P. multocida porcine strain H229 (containing a PmSLP with the sequence defined in SEQ. ID NO: 24). PmSLP-3 vaccinated mice were 100% protected. The graph shown in FIG. 6B depicts the clinical score as a function of time, notably the clinical score of mice immunized with PmSLP-3 vaccine separated by individual animal. The graph shown in FIG. 6C depicts the clinical score as a function of time, notably the clinical score of mice immunized with adjuvant only separated by individual animal. The graph shown in FIG. 6D depicts the clinical score as a function of time, notably the mean clinical score of mice from both groups; error bars depict standard error. For FIGS. 6B, 6C, and 6D, a clinical score cut off of 10 is considered the humane endpoint at which point animals are euthanized. The graph shown in FIG. 6E depicts bacterial recovery as a function of time, notably bacterial recovery from tail vein bleeds during the infection separated by individual animal. The experimental results demonstrate that PmSLP containing vaccines can be efficacious against relevant porcine P. multocida disease isolates in an acute mouse infection model.

FIGS. 7A and 7B are further graphs depicting results obtained in the performance of certain experiments, notably an experiment to evaluate the immune response in mice having been administered a PmSLP vaccine formulation. Serum samples were obtained from mice immunized with two doses of either PmSLP-3 (SEQ. ID NO: 20) vaccine or adjuvant prior to being challenged. The graph shown in FIG. 7A depicts α-PmSLP-3 IgG titre against the purified antigen as measured by protein ELISA. The graph shown in FIG. 7B depicts α-PmSLP-3 IgG titre against whole bacteria as measured by heat inactivated whole cell ELISA. P. multocida porcine isolate of P. multocida strain H229, which harbours a pmSLP gene from the same phylogenetic cluster as the vaccine antigen was used (the confirmed sequence of the PmSLP in this strain is defined in SEQ. ID NO: 24). For both FIG. 7A and FIG. 7B, individual points depict individual animals. Bars depict mean, error bars depict standard error. The experimental results demonstrate that α-PmSLP-3 antibodies elicited in vaccinated mice can bind purified antigen as well as the antigen on the bacterial surface.

FIGS. 8A and 8B are further graphs depicting results obtained in the performance of certain experiments, notably an experiment to evaluate the stability of a PmSLP polypeptide. The intrinsic fluorescence given off by tryptophan and tyrosine residues (Ratio 350 nm/330 nm) (graphs shown in FIG. 8A) was measured to generate thermal profiles and calculate the thermal inflection temperatures (Ti) (graph shown in FIG. 8B) for PmSLP-1 protein samples stored under the indicated storage conditions. The results demonstrate that purified PmSLP-3 protein (SEQ. ID NO: 20) can be stable under various storage conditions after lyophilization for one year.

FIGS. 9A, 9B, 9C, 9D, and 9E are further graphs depicting results obtained in the performance of certain experiments, notably an experiment to evaluate the protection from lethal challenge in mice having been administered PmSLP vaccine formulations. The graph shown in FIG. 9A depicts the percentage survival as a function of time, notably the percentage survival of mice after infection with antigen-matched P. multocida porcine strain H229 (the confirmed sequence of the PmSLP of this strain is defined in SEQ. ID NO: 24) wherein all mice that received PmSLP-3 vaccine formulations were fully protected from lethal challenge. The graphs shown in FIGS. 9B, 9C, 9D, and 9E show the clinical score for individual mice as a function of time, notably the clinical scores of mice immunized with Adjuvant, Vaccine 1 (freshly formulated vaccine with protein stored at −80° C. until formulation), Vaccine 2 (freshly formulated vaccine with lyophilized protein stored at 4° C. until formulation), and Vaccine 3 (formulated vaccine prepared prior to dose 1 and stored at 4° C. until dose 2) respectively. Each line depicts the clinical score of an individual mouse over the duration of the experimental challenge with clinical monitoring performed at multiple timepoints over the 36 hours post infection. The dotted line at clinical score 10 depicts clinical score cut off which is considered the humane endpoint at which point animals are euthanized. The experimental results demonstrate that PmSLP-3 (SEQ. ID NO: 20) vaccine preparations, stored under various conditions, can be efficacious in a mouse model.

FIG. 10 is a further graph depicting results obtained in the performance of certain experiments, notably an experiment to evaluate the immune response in a ruminant (bovine) species having been administered a PmSLP vaccine formulation. In this example, Zebu cattle were immunized sub-cutaneously with either PmSLP-1 (SEQ. ID NO: 14) vaccine or adjuvant. Serum samples obtained at baseline or 2-3 weeks after the indicated dose and assayed using protein-based ELISA. Data points represent individual animal, bars represent the mean α-PmSLP-1 IgG titre, error bars represent standard error. The experimental results demonstrate that a PmSLP vaccine can be immunogenic in an animal host species that is relevant in the food production industry and affected by P. multocida infections.

FIG. 11 is a further graph depicting results obtained in the performance of certain experiments, notably an experiment to evaluate the immune response in a ruminant (bovine species) having been administered a PmSLP vaccine formulation. In this example, beef cattle were immunized via intramuscular route with either PmSLP-1 (SEQ. ID NO: 14) vaccine or adjuvant. Serum samples obtained at baseline or 2-3 weeks after the indicated dose and assayed using protein-based ELISA. Data points represent individual animal, bars represent the mean α-PmSLP-1 IgG titre, error bars represent standard error. The experimental results demonstrate that a PmSLP vaccine can be immunogenic in an animal host species that is relevant in the food production industry and affected by P. multocida infections.

FIGS. 12A, 12B, and 12C are further graphs depicting results obtained in the performance of certain experiments, notably an experiment to evaluate the immune response in a ruminant (bovine) species having been administered a PmSLP vaccine formulation. In this example, zebu cattle were immunized sub-cutaneously with PmSLP-3 (SEQ. ID NO: 20) formulated with two different adjuvants in a prime-boost schedule. The graph shown in FIG. 12A demonstrates serum samples obtained at baseline, prior to the booster dose, and prior to challenge and assayed using protein-based ELISA. Data points represent individual animals, bars represent the mean α-PmSLP-3 IgG titre, and error bars represent the standard error. The graph shown in FIG. 12B shows animal survival as a function of time, notably animal survival following lethal challenge. The graph shown in FIG. 12C documents any local reactogenicity that was evaluated following administration of either vaccine dose. The example experimental results demonstrate that a PmSLP vaccine can be immunogenic and safe in an animal host species that is relevant in the food production industry and affected by P. multocida infections, as well as that those vaccine formulations can be protective against experimental challenge of a lethal dose of P. multocida.

FIGS. 13A and 13B are a diagram illustrating certain experimental techniques (FIG. 13A) and a graph representing certain results obtained in the performance of certain experimental evaluations (FIG. 13B), notably, a diagram illustrating the experimental groups formed to determine the lethal dose of 3 clinical strains of Pasteurella multocida serogroup A (AFK 300/23, 143/23 and 283/23), all of which were isolated from pigs with severe lung disease, all of which possessing a pfhA gene, a virulence marker for P. multocida (FIG. 13A); and a graph representing results relating to the survival analysis of mice challenged with 3 different strains of Pasteurella multocida serogroup A. Mice of each group (n=5) were challenged intraperitoneally with 0.2 mL of PBS containing 10², 10³ or 10⁴ bacteria. The number of dead/alive mice was monitored and is represented as the percentile of survival (FIG. 13B).

FIGS. 14A and 14B are graphs representing certain results obtained in the performance of certain experimental evaluations, notably, a graph representing results relating to the titration of murine anti-PmSLP-2 IgG, wherein aera samples collected from two experimental groups were titrated in an Indirect ELISA based on the PmSLP 2 recombinant protein, and statistical analysis was performed using the ANOVA test, wherein statistical differences (p<0.05), when existing, are indicated (FIG. 14A); and a graph representing survival analysis of mice immunized with PmSLP2-based vaccine and challenged with Pasteurella multocida A strain AFK 143/23, wherein strain AFK 143/23 expresses PmSLP cluster 2, therefore this challenge is considered homologous (FIG. 14B).

FIG. 15 is a graph representing certain results obtained in the performance of certain experimental evaluations, notably, a graph representing results relating to the survival analysis of pigs challenged with 2 different strains of Pasteurella multocida serogroup A, wherein pigs of each group (n=3) were challenged intranasally with 3 mL of PBS containing 109 or 108 bacteria, and the number of dead/alive pigs was monitored and is represented in the graph as the percentile of survival.

FIGS. 16A, 16B, and 16C are a diagram illustrating certain experimental techniques (FIG. 16A) and a graphs representing certain results obtained in the performance of certain experimental evaluations (FIGS. 16B and 16C), notably, a diagram illustrating an Immunization protocol, used to immunize groups of 4 mice each with different PmSLP-based vaccines or inoculated with sterile PBS at experimental day 0 and 14, and to challenge, at experimental day 28, all mice with P. multocida (102 bacteria/mouse) to evaluate homologous protection (FIG. 16A); a graph representing results relating to the titration of murine anti-PmSLP-1 and PmSLP-4.2 IgG, wherein sera samples collected from four experimental groups were titrated in an Indirect ELISA based on the PmSLP-1 or PmSLP-4.2 recombinant proteins, and wherein statistical analysis was performed using the two-way ANOVA test, and wherein Statistical differences (p<0.05), when existing, are indicated (FIG. 16B); and two graphs representing results relating to survival analysis of mice immunized and challenged with 2 different strains of Pasteurella multocida serogroup A, wherein the graph in Panel (I) shows a first graph representing results of an experiment wherein mice (n=8) of each group (n=2) were challenged intraperitoneally with 0.2 mL of PBS containing 102 P. multocida A, AFK 300/23 strain, and wherein the graph in Panel (II) shows a second graph representing results of an experiment wherein mice (n=8) of each group (n=2) were challenged intraperitoneally with 0.2 mL of PBS containing 102 P. multocida A, AFK 283/23 strain, and wherein, in each case, the number of dead/alive mice was monitored and is represented in the graphs as the percentile of survival, and the different groups and strains used are indicated the challenges are described in Panel (I) and Panel (II) (FIG. 16C).

The figures together with the following detailed description make apparent to those skilled in the art how the disclosure may be implemented in practice.

### DETAILED DESCRIPTION

Various compositions, methods, or processes will be described below to provide an example of an embodiment of each claimed subject matter. No embodiment described below limits any claimed subject matter and any claimed subject matter may cover processes, compositions or methods that differ from those described below. The claimed subject matter is not limited to compositions, processes or methods having all of the features of any one composition, method or process described below or to features common to multiple or all of the compositions, methods or processes described below. It is possible that a composition, method, or process described below is not an embodiment of any claimed subject matter. Any subject matter disclosed in a composition, method, or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either”.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore, any range of values described herein is intended to specifically include the limiting values of the range, and any intermediate value or sub-range within the given range, and all such intermediate values and sub-ranges are individually and specifically disclosed (e.g., a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only and is not intended to limit the scope of the present invention, which is defined solely by the claims.

All publications, patents and patent applications are herein incorporated by reference in their entirety to the same extent as if each individual publication, patent, or patent application was specifically and individually indicated to be incorporated by reference in its entirety.

### Terms and Definitions

The terms “nucleic acid”, or “nucleic acid sequence”, as used herein, refer to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acids of the present disclosure may be deoxyribonucleic nucleic acids (DNA) or ribonucleic acids (RNA) and may include naturally occurring bases including adenine, guanine, cytosine, thymidine, and uracil. The nucleic acids may also contain modified bases. Examples of such modified bases include aza and deaza adenine, guanine, cytosine, thymidine and uracil, and xanthine and hypoxanthine. A sequence of nucleotide or nucleoside monomers may be referred to as a polynucleotide sequence, nucleic acid sequence, a nucleotide sequence, or a nucleoside sequence.

The terms “polypeptide” and “protein”, as may be used interchangeably herein, in conjunction with a reference SEQ. ID NO, refer to any and all polypeptides and proteins comprising a sequence of amino acid residues which is (i) substantially identical to the amino acid sequence constituting the polypeptide having such reference SEQ. ID NO, or (ii) encoded by a nucleic acid sequence capable of hybridizing under at least moderately stringent conditions to any nucleic acid sequence encoding the polypeptide having such reference SEQ. ID NO, but for the use of synonymous codons. A sequence of amino acid residues may be referred to as an amino acid sequence, or polypeptide sequence.

The terms “nucleic acid sequence encoding a polypeptide” and “nucleic acid sequence encoding a protein”, as used herein in conjunction with a reference SEQ. ID NO, refer to any and all nucleic acid sequences encoding a polypeptide or protein having such reference SEQ. ID NO. Nucleic acid sequences encoding a polypeptide, in conjunction with a reference SEQ. ID NO, further include any and all nucleic acid sequences which (i) encode polypeptides that are substantially identical to the polypeptide having such reference SEQ. ID NO; or (ii) hybridize to any nucleic acid sequences encoding polypeptides having such reference SEQ. ID NO under at least moderately stringent hybridization conditions or which would hybridize thereto under at least moderately stringent conditions but for the use of synonymous codons.

The terms “nucleic acid sequence encoding PmSLP”, and “nucleic acid sequence encoding a “PmSLP polypeptide”, “nucleic acid sequence encoding a PmSLP protein” as may be used interchangeably herein, refer to any and all nucleic acid sequences encoding a PmSLP polypeptide, including, for example, SEQ. ID NO: 1. Nucleic acid sequences encoding a PmSLP polypeptide further include any and all nucleic acid sequences which (i) encode polypeptides that are substantially identical to the PmSLP polypeptide sequences set forth herein; or (ii) hybridize to any PmSLP nucleic acid sequences set forth herein under at least moderately stringent hybridization conditions or which would hybridize thereto under at least moderately stringent conditions but for the use of synonymous codons.

The terms “PmSLP protein” or “PmSLP polypeptide”, as may be used herein, interchangeably refer to any and all protein comprising a sequence of amino acid residues which is (i) substantially identical to the amino acid sequences constituting any PmSLP polypeptide set forth herein, including, for example, SEQ. ID NO: 2, or (ii) encoded by a nucleic acid sequence capable of hybridizing under at least moderately stringent conditions to any nucleic acid sequence encoding any PmSLP protein set forth herein, but for the use of synonymous codons. PmSLP proteins may also be numbered in order to facilitate distinguishing different PmSLP proteins referred to herein, e.g., PmSLP-1, PmSLP-2, PmSLP-3, and so on.

By the term “substantially identical” it is meant that two amino acid sequences preferably are at least 70% identical, and more preferably are at least 85% or 90% identical, and most preferably at least 95% identical, for example 96%, 97%, 98%, 99%, 99.5%, 99.6%, 99.7%, 99.8%, or 99.9% identical. In order to determine the percentage of identity between two amino acid sequences the amino acid sequences of such two sequences are aligned, using for example the alignment method of Needleman and Wunsch (J. Mol. Biol., 1970, 48:443), as revised by Smith and Waterman (Adv. Appl. Math., 1981, 2:482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12:387) BLASTP, BLASTN and FASTA (Altschul et al., J. Mol. Biol., 1990:215:403). A particularly preferred method for determining the percentage identity between two polypeptides involves the Clustal W algorithm (Thompson, J. D., Higgines, D. G. and Gibson T. J/, 1994, Nucleic Acid Res 22 (22): 4673-4680 together with the BLOSUM 62 scoring matrix (Henikoff S & Henikoff, J G, 1992, Proc. Natl. Acad. Sci. USA 89:10915-10919 using a gap opening penalty of 10 and a gap extension penalty of 0.1, so that the highest order match obtained between two sequences wherein at least 50% of the total length of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g., 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41 (% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation)−5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts, and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptide set forth in SEQ. ID NO: 2, refers to a polypeptide capable of performing the same function as the polypeptide set forth in SEQ. ID NO: 2. Functional variants include modified a polypeptide wherein, relative to a noted reference polypeptide, the modification includes a substitution, deletion, or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine, and valine, (iv) asparagine and glutamine, and (v) tryptophan, tyrosine, and phenylalanine.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example, a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

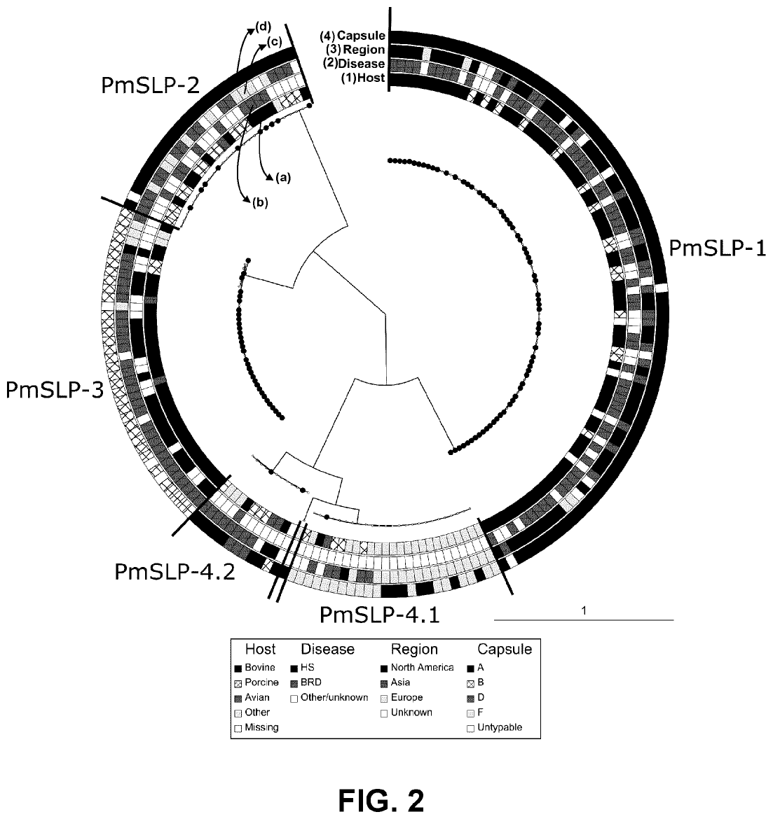

The term “phylogenetic cluster” refers to a group of evolutionary related polypeptide sequences. In order to determine whether two polypeptides belong to the same phylogenetic cluster, an evolutionary tree containing multiple branches (for example, at least 5, 7, 10, 15, or 20 branches), can be constructed using multiple more or less similar polypeptide sequences (for example, preferably at least 25, at least 50, at least 100, or at least 1,000, polypeptide sequences). Upon inspection of the phylogenetic tree, the evolutionary relationship of the polypeptide sequences can be evaluated. Polypeptides belonging to the same phylogenetic cluster are polypeptides located on a particular branch descended from a common ancestor on a phylogenetic tree. Those skilled in the art will be familiar with software programs to assist in the automated generation of phylogenetic trees based on polypeptide sequence input. Suitable phylogenetic tree construction software includes, for example, sequence alignment software such as MAFFT (v7.450) (Katoh, K. et al., 2002, Nucleic Acids Research, 30 (14), 3059-3066) which can, for example, be using the G-INS-I algorithm; evolutionary modeling software to identify an appropriate evolutionary model such as ProtTest (v3.4.2) (Darriba D, Taboada G L, Doallo R, Posada D. ProtTest 3: fast selection of best-fit models of protein evolution. Bioinformatics, 27:1164-1165, 2011); and phylogenetic tree assembly software such as PhyML (v3.3.20190909) (Guindon S., et al., 2010, Systematic Biology, 59 (3): 307-21, 2010) and RAxML (Stamakis, A. Bioinformatics, Volume 30, Issue 9, May 2014, Pages 1312-1313, doi.org/10.1093/bioinformatics/btu033). An example phylogenetic tree of PmSLP polypeptide sequences showing example phylogenetic clusters PmSLP-1, PmSLP-2, PmSLP-3, PmSLP-4.1, and PmSLP-4.2 includes the phylogenetic tree shown in FIG. 2. Example phylogenetic cluster PmSLP-1 includes, for example, at least a PmSLP polypeptide sequence having SEQ. ID NO: 2. Example phylogenetic cluster PmSLP-2 includes, for example, at least a PmSLP polypeptide sequence having SEQ. ID NO: 4. Example phylogenetic cluster PmSLP-3 includes, for example, at least a PmSLP polypeptide sequence having SEQ. ID NO: 6. Example phylogenetic cluster PmSLP-4.1 includes, for example, at least a PmSLP polypeptide sequence having SEQ. ID NO: 8. Example phylogenetic cluster PmSLP-4.2 includes, for example, at least a PmSLP polypeptide sequence having SEQ. ID NO: 10.

The term “animal”, as used herein, refers to all species belonging to the kingdom Animalia, excluding humans.

The term “avian”, as used herein, refers to any animal species belonging to the class Aves, including, for example, poultry, such as chickens, turkeys, geese, ducks, and quails.

The term “bovine”, as used herein, refers to any animal species belonging to the subfamily Bovinae including, for example, cows, cattle, oxen, bison, and buffalo.

The term “porcine”, as used herein, refers to any animal species belonging to the family Suidae including, for example, pigs, hogs, and boars.

The term “ruminant”, as used herein, refers to herbivorous mammalian animals having a digestive system comprising multi-compartment stomach system, including typically a rumen, reticulum, omasum, and abomasum, capable of digesting plant materials through microbial fermentation processes. Ruminants include cows, cattle, oxen, goats, sheep, bison, and buffalo.

The term “food production animal”, as used herein, refers to animals raised and farmed by humans for human food production. Food production animals include, without limitation, ruminant species, bovine species, porcine species, and avian species.

The terms “Pasteurella multocida” or “P. multocida”, as used herein, refer to any bacteria belonging the bacterial species taxonomically classified as such and include any subspecies thereof, including P. multocida subspecies multocida, P. multocida subspecies gallicida, and P. multocida subspecies septica, and further include any P. multocida strains, variants, serogroups (including serogroups A, B, D, E and F), serotypes (including serotypes 1-16), or genotypes. It is noted that P. multocida strains may be referred to by serogroup and serotype. Thus, for example, a strain referred to as P. multocida A: 3, denotes a P. multocida strain of serogroup A and serotype 3.

The term “effective amount”, as used herein, refers to an amount of an active agent or veterinary pharmaceutical formulation, including a veterinary vaccine formulation, sufficient to induce a desired biological or therapeutic effect, including a prophylactic effect. Such effect can include an effect with respect to the signs, symptoms or causes of a disorder, or disease or any other desired alteration of a biological system. The effective amount can vary depending, for example, on the health condition, injury stage, disorder stage, or disease stage, of the animal being treated, timing of the administration, manner of the administration, age of the animal, size of the animal, and the like, all of which can be determined by those of skill in the art.

The term “immunologically equivalent”, as used herein, refers to a molecule that is capable of eliciting a humoral immune response in the form of the production of native polyclonal antibodies in a subject animal when administered thereto, wherein the binding specificity to the native polyclonal antibodies is comparable to the specificity of native polyclonal antibodies produced when a reference molecule is administered to the subject animal. For example, immunologically equivalent portions of a reference full length PmSLP polypeptide include immunogenic portions of a PmSLP polypeptide which when administered to a subject animal elicit a humoral immune response in the form of the production of native antibodies with a specificity to a PmSLP polypeptide which is comparable to the binding specificity for a PmSLP polypeptide of native antibodies obtained when the reference full length PmSLP is administered to the animal. Immunologically equivalent portions of full length PmSLP polypeptides can vary in length and may, for example, include polypeptides comprising or consisting of at least 10, at least or up to 15, at least or up to 20, at least or up to 30, at least or up to 50, or at least or up to 60 consecutive amino acid residues which are identical to a portion of a PmSLP polypeptide. Furthermore, immunologically equivalent portions of full length PmSLP polypeptides include polypeptides which are at least 90%, at least 95%, at least 96%, at least 97%, at least 98%, or at least 99%, at least 99.5%, at least 99.6%, at least 99.7%, at least 99.8%, at least 99.9% identical to a full length PmSLP polypeptide. To compare binding specificity between a reference molecule and an immunologically equivalent molecule a radioimmune assay (RIA) may be used, and the extent of binding may be measured. The dissociation constant of an immunologically equivalent molecule is preferably at least 50%, at least 60%, at least 70%, at least 80%, at least 90%, or at least 95% of the dissociation constant of a reference molecule.

The term “cross-protective”, as used herein, refers to a vaccine formulation capable of providing protection against infection by multiple strains (e.g., 2, 3, 4, 5, or more strains) of a pathogenic microbial organism, for example, strains belonging to multiple serogroups or serotypes. A cross-protective vaccine formulation may comprise multiple antigenic substances, or a single antigenic substance, for example, multiple immunogenic polypeptides or a single immunogenic polypeptide. To evaluate cross-protection, a vaccine formulation comprising an immunogenic substance obtained from a selected strain of a microbial organism, a PmSLP protein, for example, may be used to immunize a subject animal. The subject animal then may be exposed to (challenged with) another pathogenic strain of the pathogenic microbial organism, and the immunological response and the development of disease symptoms of the animal may be evaluated. In the event the infection results in the development of an improved immune response, or less severe or no disease symptoms in the infected animal than in an unvaccinated animal exposed to the same infection, the vaccine formulation can be said to be cross-protective.

The term “veterinary pharmaceutically acceptable”, as used herein, refers to materials, including carriers, diluents, or auxiliary agent that are compatible with other materials in a veterinary pharmaceutical formulation, including a veterinary vaccine formulation, and within the scope of reasonable medical judgement suitable for use in contact with animals without excessive toxicity, allergic response, irritation, or other adverse response commensurate with a reasonable risk/benefit ratio.

The terms “treating” and “treatment”, and the like, as used herein, are intended to mean obtaining a desirable physiological, pharmacological, or biological effect. The effect may result in the prevention (i.e., prophylactic treatment), inhibition, amelioration, attenuation, reversal of a sign, symptom or cause of a disorder, or disease, attributable to the disorder, or disease. Clinical evidence of the treatment may vary with the disorder, or disease, the animal, and the selected treatment. In the context of the treatment of an indication a physiological effect may include, for example, an improved respiratory capacity or lung function, reduced hemorrhaging, reduced mucoid nasal or oral discharge, improved rectal temperatures, improved animal demeanor, reduced coughing, or a reduction in lung lesions.

The term “respiratory disease”, as used herein refers to the accepted veterinary medical definition of a respiratory disease and includes any disease involving invasion and colonization by P. multocida species of the respiratory tract, including the upper and lower respiratory tract of an animal, and includes, for example, BRD and HS.

The terms “bovine respiratory disease” or “BRD”, as used herein, refer to the accepted medical veterinary definition of bovine respiratory disease, and include, in general, a respiratory disease state in a bovine animal caused by a pathogenic P. multocida infection.

The terms “hemorrhagic septicemia” or “HS”, as used herein, refer to refer to the accepted medical veterinary definition of hemorrhagic septicemia, and include, in general, a respiratory disease state in a bovine animal caused by a pathogenic P. multocida infection.

The term “porcine atrophic rhinitis” or “PAR”, as used herein, refer to refer to the accepted medical veterinary definition of porcine atrophic rhinitis and includes, in general, a respiratory disease state in a porcine animal caused by a pathogenic P. multocida infection.

The term “pneumonic pasteurellosis”, as used herein, refers to the accepted medical veterinary definition of as used herein, refer to refer to the accepted medical veterinary definition of pneumonic pasteurellosis and includes, in general, a respiratory disease state in a porcine animal caused by a pathogenic P. multocida infection.

The term “fowl cholera”, as used herein, refers to refer to the accepted medical veterinary definition of fowl cholera, and includes, in general, a respiratory disease state in an avian animal caused by a pathogenic P. multocida infection.

The terms “vaccine”, “vaccine formulation”, “veterinary vaccine”, and “veterinary vaccine formulation” as used herein, refer to a veterinary pharmaceutically acceptable preparation that may be administered to an animal to induce a humoral immune response (including eliciting a soluble antibody response) and/or cell-mediated immune response (including eliciting a cytotoxic T lymphocyte (CTL) response).

The term “homologous protection”, as used herein, refers to protection conferred by a vaccine against infection by a strain of a microbial species, wherein the infecting strain possesses an antigen, for example a PmSLP protein antigen, which belongs to the same phylogenetic cluster as an antigen, for example, a PmSLP protein antigen, or an immunologically equivalent portion thereof, included in the vaccine formulation.

The term “heterologous protection”, as used herein, refers to protection conferred by a vaccine against infection by a strain of a microbial species, wherein the infecting strain possesses an antigen, for example a PmSLP protein antigen, which belongs to a different phylogenetic cluster as an antigen, for example, a PmSLP protein antigen, or an immunologically equivalent portion thereof, included in the vaccine formulation.

The terms “substantially pure” and “isolated”, as may be used interchangeably herein describe a compound, e.g., a polypeptide, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides, by chromatography, gel electrophoresis or HPLC analysis.

### General Implementation

As hereinbefore mentioned, the present disclosure relates to veterinary vaccine formulations. In general, the herein provided methods and compositions can be used to prevent or ameliorate Pasteurella multocida infections in food production animals, including ruminant animal species, bovine animal species, porcine animal species, and avian animal species. In this respect, the methods, and compositions of the present disclosure, in particular, may be used to treat bovine respiratory disease or hemorrhagic septicemia in bovine species, pneumonic pasteurellosis or progressive atrophic rhinitis in swine, or fowl cholera in poultry. The veterinary vaccine formulations of the present disclosure can provide long-term, effective protection against P. multocida infections.

The inventors have discovered that the veterinary vaccine formulations of the present disclosure are cross-protective and can provide protection to food production animals against infection by multiple P. multocida strains. Surprisingly, the veterinary vaccine formulations of the present disclosure can be cross-protective using a single immunogenic active agent.

Furthermore, the vaccine formulations of the present disclosure can provide long-term protection, with antibodies against the immunizing agent being detectable in the blood serum of a subject food production animal, for example, at least 26 weeks from administration of the vaccine formulation.

Furthermore, the vaccine formulations of the present disclosure involve the use of polypeptide-based immunogenic active agents, and as such, the compositions and methods of the present disclosure do not involve the use of live attenuated microbial species, and thus avoid infection risks associated with the use of live vaccines.

Furthermore, the vaccine formulations of the present disclosure can limit the administration of antibiotics to food production animals, and thus limit the development of antibiotic resistant microbial strains. In addition, some consumers have a preference for food products obtained from animals which have not been treated with antibiotics.

Furthermore, the polypeptide-based immunogenic active agents included in the vaccine formulations of the present disclosure may be prepared using a convenient recombinant production system, and the immunogenic active agents may be stably stored.

The present inventors have discovered, in particular, that proteins selected from a class of proteins known as PmSLP proteins may be used as immunogenic active agents in the formulation of veterinary vaccines to prevent or ameliorate P. multocida infections in food production animals. The PmSLP proteins can be selected and obtained from a P. multocida strain. The P. multocida strain from which the PmSLP protein is selected preferably is a P. multocida strain which belongs to the same phylogenetic cluster as the P. multocida strain causing the infection in the food production animal. Thus, the PmSLP protein does not need to be selected from the same P. multocida strain as the infecting P. multocida infecting strain provided however, said another strain belongs to the same phylogenetic cluster.

In what follows example embodiments of the compositions and methods of the present disclosure are described.

Thus, the present disclosure provides, in at least one aspect, and in at least one embodiment, a veterinary vaccine formulation for the prevention or amelioration of P. multocida infection in a food production animal susceptible to P. multocida infection, the vaccine formulation comprising an effective amount of a PmSLP protein or an immunogenically equivalent portion thereof.

In another aspect, and in at least one other embodiment, the present disclosure further provides a method for treatment of a food production animal susceptible to P. multocida infection, the method comprising administering to the food production animal a veterinary vaccine formulation comprising a PmSLP protein or an immunogenically equivalent portion thereof, wherein the vaccine formulation is administered in an effective amount to prevent or ameliorate the P. multocida infection.

In general, according to an aspect, veterinary vaccine formulations comprising a selected PmSLP protein or an immunogenically equivalent portion thereof are prepared or obtained. These formulations are administered in effective amounts to a food production animal in need thereof. Thus, in what follows next suitable preparations comprising a selected PmSLP protein, or an immunogenically equivalent portion thereof, will be described, as well as methods of making a selected PmSLP protein, or an immunogenically equivalent portion thereof. Thereafter, veterinary vaccine formulations comprising a selected PmSLP protein or an immunogenically equivalent portion thereof, and methods of preparing veterinary vaccine formulations and administering the same to a food production animal in need thereof will be described.

Thus, initially, considering PmSLP protein preparations, in an aspect, preparations containing a selected PmSLP protein or an immunogenically equivalent portion thereof can be prepared biosynthetically using a host cell system. In this respect, an isolated nucleic acid encoding an amino acid sequence corresponding with a PmSLP protein or an immunogenically equivalent portion thereof can be introduced in host cells and expressed therein.

In general, any PmSLP protein may be used in accordance herewith. PmSLP proteins can be obtained from P. multocida bacteria, including any PmSLP comprising strain thereof, including any PmSLP comprising P. multocida strain belonging to any serogroup or serotype. In this respect, it is noted, as is known to those of skill in the art, that P. multocida strains may be classified as belonging to different serogroups and/or serotypes. Serogroups in this respect include serogroups A, B, D, E, and F, and serotypes include 1, 2, 3, 4, 5, 6, 7, 8, 9, 10, 11, 12, 13, 14, 15, and 16. When referring to different P. multocida strains, serogroups and serotypes may be referred to accordingly. For example, P. multocida strain A: 3 can be classified as belonging to serogroup A and serotype 3, P. multocida strain E: 2 strain can be classified as belonging to serogroup E and serotype 2, and so on. Thus, in accordance herewith the PmSLP protein can be a PmSLP protein obtained from a P. multocida strain selected from serogroup A, B, D, E, and F P. multocida strains. Furthermore, the PmSLP protein can be a PmSLP protein obtained from a P. multocida strain selected from serotype 1, 2, 3, 4, 5, 6, 7, 8, 9, 10, 11, 12, 13, 14, 15, and 16 P. multocida strains. Thus, it is to be understood that the PmSLP protein, in accordance herewith, can be selected from PmSLP proteins obtainable or obtained from a P. multocida strain belonging to any serogroup or any serotype, or any combination thereof.

According to an aspect, in an example embodiment, a nucleic acid sequence encoding a PmSLP protein may be selected, wherein such nucleic acid includes SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96 set forth herein. Selected example PmSLP proteins include SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, which are polypeptides encoded by SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96, respectively.

In some embodiments, the PmSLP protein can be a protein expressed by a nucleic acid sequence selected from the group of nucleic acid sequences consisting of
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(f) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof; and
(g) a nucleic acid sequence that hybridizes under stringent conditions to any one of the nucleic acid sequences set forth in (a), (b), (c), (d), (e) or (f).

PmSLP proteins that may be used in accordance herewith include naturally occurring PmSLP proteins, as well as PmSLP proteins that may have been modified. Modifications in this respect include modifications made to the amino acid sequence of a PmSLP polypeptide, including for example, a modification in one or more specific individual amino acids, which may be referred to as “site-directed mutations”, such as for example a PmSLP protein having SEQ. ID NO: 65 and SEQ. ID NO: 67.

Further modifications that may be made to PmSLP proteins include modifications which diminish binding of the PmSLP protein to a native host protein, (i.e., a protein in a food production animal susceptible to P. multocida infection). Such diminished binding can generally be assessed by evaluating the affinity of a PmSLP protein for a native host protein. The affinity may be quantitatively evaluated by experimentally determining the dissociation constant (Kd) between the PmSLP protein and the native host protein. Thus, for example, in this respect, the Kd between a native PmSLP protein and a native host protein, for example between PmSLP-1 and bovine complement factor I, may be compared with the Kd between a modified PmSLP protein and the same native host protein. In general, the higher the Kd value the weaker the affinity of the PmSLP protein for the native host protein. In some embodiments, the Kd between a modified PmSLP and a native host protein may exceed the Kd between the native non-modified PmSLP and the native host protein by a factor of at least 2×, at least 5×, at least 10×, at least 25×, at least 50×, at least 100×, at least 250×, or at least 500×. Techniques to determine the Kd between two proteins are well known to those of skill in the art and include, for example isothermal calorimetry, surface plasmon resonance and biolayer interferometry (see further, for example, Rich R. et al. 2007, Anal. Biochem 361:1-6; Abdiche, Y. et al., 2008, Anal. Biochem. 377:209-217; and Velazquez-Campoy, A. et al., 2004, Methods Mol. Biol. 261:35-54).

Examples of PmSLP polypeptides that may be used in this respect include PmSLP polypeptides having SEQ. ID NO: 65 and SEQ. ID NO: 67 (encoded by nucleic acid sequences having SEQ. ID NO: 64 and SEQ. ID NO: 66, respectively). In this respect, the PmSLP polypeptides having SEQ. ID NO: 65, and SEQ. ID NO: 67 encode PmSLP-1 polypeptides (SEQ. ID NO: 2) comprising a V214D mutation (substitution of valine amino acid residue 214 by an aspartic acid amino acid residue), and E240A mutation (substitution of glutamic acid amino acid residue 240 by an alanine acid amino acid residue). The Kd between each modified PmSLP having SEQ. ID NO: 65, and SEQ. ID NO: 67 and a native host protein was determined to be less than 2,000 nM, with the Kd between the wild type PmSLP-1 protein (SEQ. ID NO: 2) and the native host protein being 30 nM±10 nM. Thus, the Kd between these two example modified PmSLP proteins and a native host protein exceeds the Kd between the native non-modified PmSLP and the native host protein by a factor of at least 500×.

According to an aspect, suitable nucleic acid sequences include nucleic acid sequences encoding an immunogenically equivalent portion of a PmSLP polypeptide, notably, in particular, PmSLP polypeptide portions which are at least immunologically equivalent to full length PmSLP polypeptides, including the polypeptides set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97. Thus, further example nucleic acid sequences that may be used in selected embodiments include nucleic acid sequences encoding an amino acid sequence which is at least immunologically equivalent to a PmSLP polypeptide, the amino acid sequence corresponding with at least 10 consecutive amino acids and up to 150 amino acids, including 10, 20, 30, 40, 50, 55, 60, 70, 80, 90, 100, 110, 120, 130, 140, or 150 amino acids of a full length PmSLP polypeptide, including the PmSLP polypeptides set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97.

In a further embodiment, the PmSLP protein or immunogenically equivalent portion thereof may be linked to another polypeptide to form a hybrid polypeptide, including, for example a carrier protein or extension to facilitate detection or purification, such as a polyhistidine extension (HIS-tag) or a FLAG-tag peptide extension, or another immunogenic polypeptide, including, further a second PmSLP or an immunogenically equivalent portion thereof.

Further examples of hybrid polypeptides, are hybrid polypeptides comprising a first and second PmSLP polypeptide, for example, PmSLP-1 and PmSLP-3, and PmSLP-1 and PmSLP-2, such as set forth in SEQ. ID NO: 51 (PmSLP-1 and PmSLP-2 hybrid), SEQ. ID NO: 53 (PmSLP-1, PmSLP-2, PmSLP-3 hybrid), and SEQ. ID NO: 55 (PmSLP-1, PmSLP-2, PmSLP-4.2 hybrid), which can be encoded by the nucleic acid sequences set forth in SEQ. ID NO: 50, SEQ. ID NO: 52, SEQ. ID NO; and SEQ. ID NO: 54, respectively.

Yet further examples of hybrid polypeptides are hybrid polypeptides comprising a first, second, and third PmSLP polypeptide, for example, PmSLP-1, PmSLP-2, and PmSLP-3, and PmSLP-1, PmSLP-2, and PmSLP-4, such as set forth in SEQ. ID NO: 53 (PmSLP-1, PmSLP-2, and PmSLP-3 hybrid polypeptide) and SEQ. ID NO: 55 (PmSLP-1, PmSLP-2, and PmSLP-3 hybrid polypeptide), which can be encoded by the nucleic acid sequences set forth in SEQ. ID. NO: 52 and SEQ. ID NO: 54, respectively.

As is known to those of skill in the art, expression of nucleic acids in a host cell, to produce a protein thereby biosynthetically, can be achieved by providing one or more nucleic acids capable of controlling expression in a host cell, and operably linking the one or more nucleic acids capable of controlling expression in a host cell to the nucleic acid one wishes to express. Such operable linking of a nucleic acid controlling expression generally involves linking in the 5′ to 3′ direction of expression the nucleic acid capable of controlling expression in a host cell to the nucleic acid one wishes to express, i.e., within the context of the instant disclosure, a PmSLP protein. Nucleic acid sequences capable of controlling expression in host cells that may be used herein include any transcriptional promoter capable of controlling expression of polypeptides in host cells. Generally, promoters obtained from bacterial cells are used when a bacterial host cell is selected, while a yeast promoter will be used when a yeast host cell is selected, a plant promoter will be used when a plant cell is selected, and so on. The obtained nucleic acid comprising a promoter and the nucleic acid expressing a PmSLP protein is generally a chimeric nucleic acid. Further nucleic acid elements capable elements of controlling expression in a host cell include transcriptional terminators, enhancers and the like, all of which may be included in the chimeric nucleic acid sequences of the present disclosure.

In accordance with the present disclosure, the chimeric nucleic acid sequences can be integrated into a recombinant expression vector which ensures good expression in the host cell, wherein the recombinant expression vector is suitable for expression in a host cell. The term “suitable for expression in a host cell” means that the recombinant expression vector comprises the chimeric nucleic acid linked to genetic elements required to achieve expression in a cell. As noted, such genetic elements can include transcriptional promoters, terminators, and enhancers, and the like. Further genetic elements that may be included in the expression vector are one or more nucleic acid sequences encoding marker genes, and one or more origins of replication. In some embodiments, the expression vector can freely replicate in the host cell. In other embodiments, the chimeric nucleic acid can be integrated into the host cell's genomic DNA. In some embodiments, the expression vector further can comprise genetic elements required for the integration of the vector or a portion thereof in the host cell's genome, for example, if a plant host cell is used the T-DNA left and right border sequences which facilitate the integration into the plant's nuclear genome can be included in the vector.

Marker genes that may be used in accordance with the present disclosure include all genes that allow the distinction of transformed cells from non-transformed cells, including all selectable and screenable marker genes. A marker gene may be a resistance marker such as an antibiotic resistance marker against, for example, kanamycin, chloramphenicol, methotrexate, or ampicillin. In other instances, a marker gene may be a gene which allows a cell to produce an essential nutrient, for example amino acids.

Thus, in an aspect, the present disclosure provides, in an example embodiment, an expression vector comprising:
(a) a nucleic acid sequence encoding a PmSLP protein or an immunogenically equivalent portion thereof; and
(b) a nucleic acid sequence capable of controlling expression of the nucleic acid sequence encoding the PmSLP protein or an immunogenically equivalent portion thereof in a host cell.

In example embodiments, the expression vector can comprise a chimeric nucleic acid comprising a nucleic acid sequence encoding a promoter linked to a nucleic acid sequence encoding a PmSLP protein or an immunogenically equivalent portion thereof.

Turning now to the host cell, it is noted, initially, that any host cell which upon cultivation expresses the chimeric nucleic acid can be selected and used in accordance with the present disclosure. Suitable host cells in this respect include, for example, microbial cells, such as bacterial cells, yeast cells, for example, and algal cells or plant cells. A variety of techniques and methodologies to manipulate host cells to introduce nucleic acid sequences in cells and attain expression exists and are well known to the skilled artisan. These methods include, for example, cation-based methods, for example, lithium ion or calcium ion-based methods, electroporation, biolistics, and glass beads-based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

One example host cell that conveniently may be used is Escherichia coli. The preparation of the E. coli vectors may be accomplished using commonly known techniques such as restriction digestion, ligation, gel electrophoresis, DNA sequencing, the polymerase chain reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Suitable promoter sequences for use in E. coli include, for example, the T7 promoter, the T5 promoter, tryptophan (trp) promoter, lactose (lac) promoter, tryptophan/lactose (tac) promoter, lipoprotein (lpp) promoter, and λ phage PL promoter. Typically, cloning vectors contain a marker, for example, an antibiotic resistance marker, such as ampicillin or kanamycin resistance marker, allowing selection of transformed cells. Nucleic acid sequences may be introduced in these vectors, and the vectors may be introduced in E. coli by preparing competent cells, electroporation or using other well-known methodologies to a person of skill in the art. E. coli may be grown in an appropriate medium, such as Luria-Broth medium and harvested. Recombinant expression vectors may readily be recovered from cells upon harvesting and lysing of the cells.

Another example host cell that may be conveniently used is a yeast cell. Example yeast host cells that can be used are yeast cells belonging to the genus Candida, Kluyveromyces, Saccharomyces, Schizosaccharomyces, Pichia, Hansenula, and Yarrowia. In specific example embodiments, the yeast cell can be a Saccharomyces cerevisiae cell, a Yarrowia lipolytica cell, or Pichia pastoris cell.

A number of vectors exist for the expression of recombinant proteins in yeast host cells. Examples of vectors that may be used in yeast host cells include, for example, Yip type vectors, YEp type vectors, YRp type vectors, YCp type vectors, pGPD-2, pAO815, pGAPZ, pGAPZa, PHIL-D2, PHIL-S1, pPIC3.5K, pPIC9K, pPICZ, pPICZa, pPIC3K, pHWO10, pPUZZLE and 2 μm plasmids. Such vectors are known to the art and are, for example, described in Cregg et al., Mol Biotechnol. (2000) 16 (1): 23-52. Suitable promoter sequences for use in yeast host cells are also known and described, for example, in Mattanovich et al., Methods Mol. Biol., 2012, 824:329-58, and in Romanos et al., 1992, Yeast 8:423-488. Examples of suitable promoters for use in yeast host cells include promoters of glycolytic enzymes, like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (PPGK), the glycerol aldehyde phosphate dehydrogenase promoter (PGAP), translation elongation factor promoter (PTEF), S. cerevisiae enolase (ENO-1), S. cerevisiae galactokinase (GAL1), S. cerevisiae alcohol dehydrogenase/glyceraldehyde-3-phosphate dehydrogenase (ADH1, ADH2/GAP), S. cerevisiae triose phosphate isomerase (TPI), S. cerevisiae metallothionein (CUP1), and S. cerevisiae 3-phosphoglycerate kinase (PGK), and the maltase gene promoter (MAL). Marker genes suitable for use in yeast host cells are also known to the art. Thus, antibiotic resistance markers, such as ampicillin resistance markers, can be used in yeast, as well as marker genes providing genetic functions for essential nutrients, for example, leucine (LEU2), tryptophan (TRP1 and TRP2), uracil (URA3, URA5, URA6), histidine (HIS3), and the like. Methods for introducing vectors into yeast host cells can, for example, be found in S. Kawai et al., 2010, Bioeng. Bugs 1 (6): 395-403.

Yet other example host cells that may be used in accordance herewith are plant cells. Methods for introducing nucleic acids in plant cells are known to those of skill in the art. Agrobacterium mediated plant cell transformation methods are described, for example, by Gelvin S. in Microbiol. Mol. Biol. Rev., 2003, 67 (1): 16-37, and physical transformation-based methods for plant cells are described by Rivera A. L. et al., 2012, Phys. Life Rev. 9 (3): 308-345. Plant selectable marker genes are known to those of skill in the art and include antibiotic resistance genes, for example kanamycin resistance genes, and herbicide resistance genes, such as the bar and pat genes (Wohlleben et al., 1988, Gene 70:25-37). Screenable markers that may be employed to identify plant transformants through visual inspection include β-glucuronidase (GUS) (U.S. Pat. Nos. 5,268,463 and 5,599,670) and green fluorescent protein (GFP) (Niedz et al., 1995, Plant Cell Rep., 14:403). Plant promoters are also known to those in the art and include, for example, constitutive promoters, such as the 35S cauliflower mosaic virus (CaMV) promoter (Rothstein et al., 1987, Gene 53:153-161), the rice actin promoter (McElroy et al., 1990, Plant Cell 2:163-171; U.S. Pat. No. 6,429,357), a ubiquitin promoter, such as the corn ubiquitin promoter (U.S. Pat. Nos. 5,879,903 and 5,273,894), and the parsley ubiquitin promoter (Kawalleck, P. et al., 1993, Plant Mol. Biol. 21:673-684), and organ specific promoters, such as seed specific promoters, for example, a phaseolin promoter (Sengupta-Gopalan et al., 1985, Proc. Natl. Acad. Sci. USA 82:3320-3324), or an oleosin promoter (U.S. Pat. No. 5,792,922).

Further, guidance with respect to the preparation of expression vectors and introduction thereof into host cells, including in E. coli cells, yeast cells, and other host cells, may be found in, for example: Sambrook et al., Molecular Cloning, a Laboratory Manual, Cold Spring Harbor Laboratory Press, 2012, Fourth Ed.

Thus, in another aspect, the present disclosure provides, one example embodiment, a host cell comprising a chimeric nucleic acid comprising:
(a) a nucleic acid sequence encoding a PmSLP protein or an immunogenically equivalent portion thereof; and
(b) a nucleic acid sequence capable of controlling expression of the nucleic acid sequence encoding the PmSLP protein or an immunogenically equivalent portion thereof in a host cell.

In example embodiments, the expression vector can comprise a chimeric nucleic acid comprising a nucleic acid sequence encoding a promoter linked to a nucleic acid sequence encoding a PmSLP protein or an immunogenically equivalent portion thereof.

Thus, to briefly recap, a host cell comprising a chimeric nucleic acid comprising (i) a nucleic acid sequence encoding a PmSLP protein or an immunogenically equivalent portion thereof; and (ii) a nucleic acid sequence capable of controlling expression of the nucleic acid sequence encoding a PmSLP protein or an immunogenically equivalent portion thereof in a host cell can be prepared in accordance with the present disclosure.

In accordance herewith, host cells are grown to multiply and to express a chimeric nucleic acid. Expression of the chimeric nucleic acid results in the biosynthetic production in the host cell of a PmSLP protein of an immunogenically equivalent portion thereof. Growth media and growth conditions can vary depending on the host cell that is selected, as will be readily appreciated to those of ordinary skill in the art. Growth media typically contain a carbon source, one or several nitrogen sources, essential salts including salts of potassium, sodium, magnesium, phosphate and sulphate, trace metals, water soluble vitamins, and process aids including but not limited to antifoam agents, protease inhibitors, stabilizers, ligands and inducers. Typical carbon sources are e.g., mono-or disaccharides. Typical nitrogen sources are, e.g., ammonia, urea, amino acids, yeast extract, corn steep liquor and fully or partially hydrolyzed proteins. Typical trace metals are e.g., Fe, Zn, Mn, Cu, Mo and H₃BO₃. Typical water-soluble vitamins are e.g., biotin, pantothenate, niacin, thiamine, p-aminobenzoic acid, choline, pyridoxine, folic acid, riboflavin, and ascorbic acid. Further, specific example media include liquid culture media for the growth of yeast cells and bacterial cells including, Luria-Bertani (LB) broth for bacterial cell cultivation, and yeast extract peptone dextrose (YEPD or YPD), for yeast cell cultivation. Further media and growth conditions can be found in Sambrook et al., Molecular Cloning, a Laboratory Manual, Cold Spring Harbor Laboratory Press, 2012, Fourth Ed.

Upon production by the host cells of a PmSLP protein or an immunogenically equivalent portion thereof, the PmSLP protein or the immunogenically equivalent portion thereof may be recovered from the host cells, and separated from other constituents, such as cellular debris, or media constituents, for example. Separation techniques will be known to those of skill in the art and include a variety of different protein purification techniques including, e.g., ion-exchange chromatography, size exclusion chromatography, affinity chromatography, hydrophobic interaction chromatography, reverse phase chromatography, gel filtration, etc. Further general guidance with respect to protein purification may for example be found in: Cutler, P. Protein Purification Protocols, Humana Press, 2004, Second Ed. Thus, substantially pure preparations of PmSLP proteins or immunogenically equivalent portions thereof may be obtained. The recovered PmSLP proteins may be obtained in a more or less pure form, for example, a preparation of a PmSLP protein or immunogenically equivalent portion thereof having a purity of at least about 60% (w/v), about 70% (w/v), about 80% (w/v), about 90% (w/v), about 95% (w/v), or about 99% (w/v) may be obtained.

Furthermore, it is noted that the recombinant production of the PmSLP proteins or immunogenically equivalent portions thereof in host cell system permits the production thereof in a manner in which the PmSLP proteins or immunogenically equivalent portions are substantially free from other P. multocida constituent materials, such as other P. multocida proteins, membrane materials, lipopolysaccharides, and the like, naturally associated with PmSLP proteins.

It is noted that the cells, in some embodiments, may secrete a portion of the produced PmSLP protein or immunogenically equivalent portion thereof the in the cell growth medium, thus a portion of the produced PmSLP protein or immunogenically equivalent portion thereof may be recovered from the cells and a further portion of the PmSLP protein or immunogenically equivalent portion thereof may be recovered from the growth medium.

It is further noted that the veterinary vaccine formulations of the present disclosure may comprise PmSLP proteins or an immunogenically equivalent portion thereof in more or less pure form. Thus, in accordance herewith, a substantially pure PmSLP protein or immunogenically equivalent portion thereof may be obtained and used to prepare veterinary vaccine formulations. Thus, for example, in some embodiments, the PmSLP protein may be substantially free of other host cell constituent materials, such as host cell proteins, membrane materials, lipopolysaccharides, and the like. In other embodiments, more crude preparations comprising PmSLP protein or immunogenically equivalent portion thereof may be obtained and used to prepare vaccine formulations. Thus, for example, in such embodiments, host cells, host cell lysates or host cell fractions comprising the PmSLP protein or immunogenically equivalent portion thereof may be used to prepare the vaccine formulations.

It is noted that any PmSLP or immunogenically equivalent portion thereof may be used to formulate the veterinary vaccine formulations of the present disclosure, including a PmSLP protein having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof.

Furthermore, in an aspect of the present disclosure, when preparing a vaccine formulation, preferably consideration is given to the P. multocida strain causing the infection in the food production animal, when a PmSLP protein is selected.

In particular, in preparing a vaccine formulation to treat an animal infected by a P. multocida strain possessing a PmSLP protein belonging to a specific phylogenetic cluster, the vaccine is preferably prepared by selecting a PmSLP polypeptide belonging to the same phylogenetic cluster, including the same PmSLP polypeptide or another PmSLP polypeptide belonging to the same phylogenetic cluster, and including the thus selected PmSLP polypeptides in the vaccine formulation. Thus, for example, referring to FIG. 2, in preparing a vaccine formulation, preferably, to treat an animal infected by a P. multocida strain possessing a PmSLP polypeptide belonging to a phylogenetic cluster PmSLP-1, PmSLP-2, PmSLP-3, PmSLP-4.1, or PmSLP-4.2, a PmSLP polypeptide from the same phylogenetic cluster is selected to prepare the vaccine. Thus, by way of example, if the animal is infected by a P. multocida strain possessing a PmSLP polypeptide belonging to phylogenetic cluster PmSLP-1, for example, SEQ. ID NO: 2, the vaccine formulation preferably is prepared by selecting a PmSLP polypeptide or an immunogenically equivalent portion thereof belonging to phylogenetic cluster PmSLP-1, for example, SEQ. ID NO: 2, or another PmSLP polypeptide belonging to phylogenetic cluster PmSLP-1, and included the same in the vaccine preparation; and, by way of further example, if the animal is infected by a P. multocida strain possessing a PmSLP protein belonging to a phylogenetic cluster PmSLP-3, for example, SEQ. ID NO: 6, the vaccine formulation preferably is prepared by selecting a PmSLP polypeptide or an immunogenically equivalent portion thereof, belonging to phylogenetic cluster PmSLP-3, for example SEQ. ID NO: 6, for example, SEQ. ID NO: 6, or another PmSLP polypeptide belonging to phylogenetic cluster PmSLP-3, and included the same in the vaccine preparation. The strain from which the PmSLP protein is selected can be the same P. multocida strain, or another strain than the P. multocida infecting strain, provided however, said another strain belongs to the same phylogenetic cluster. Thus, the PmSLP protein does not need to be selected from the same P. multocida strain as the infecting P. multocida infecting strain provided however, said another strain belongs to the same phylogenetic cluster.

Furthermore, preferably, in selecting a PmSLP polypeptide consideration is given to the host and clinical indication. In particular, in preparing a vaccine formulation to treat an animal host infected by a P. multocida strain causing a particular indication, the vaccine is preferably prepared by selecting a PmSLP polypeptide from a P. multocida strain capable of causing the indication in the host, including the same PmSLP polypeptide or another PmSLP polypeptide, isolated from a P. multocida strain capable of causing the indication in the host, and including the thus selected PmSLP polypeptides in the vaccine formulation. Thus, for example, referring again to FIG. 2, in preparing a vaccine formulation, preferably, to treat an animal infected by a P. multocida strain possessing a PmSLP polypeptide belonging to a phylogenetic cluster PmSLP-1, PmSLP-2, PmSLP-3, PmSLP-4.1, or PmSLP-4.2, a PmSLP polypeptide from the same phylogenetic cluster is selected to prepare the vaccine, wherein furthermore the PmSLP is obtainable from a P. multocida strain, known to cause the indication to be treated in the host species to be treated. Thus, by way of example, if a bovine animal is diagnosed with BRD caused by infection of a P. multocida strain possessing a PmSLP polypeptide belonging to phylogenetic cluster PmSLP-2, for example, SEQ. ID NO: 4, the vaccine formulation preferably is prepared by selecting a PmSLP polypeptide or an immunogenically equivalent portion thereof belonging to phylogenetic cluster PmSLP-2, for example, SEQ. ID NO: 4, or another PmSLP polypeptide belonging to phylogenetic cluster PmSLP-2, wherein further the PmSLP-2 polypeptide further preferably is obtainable from a P. multocida strain known to cause BRD in bovine animals, and not, for example, from porcine animals, which also can be infected by P. multocida strains contain PmSLP-2, as can be appreciated from FIG. 2.

In one example embodiment, the food production animal can be a ruminant animal susceptible to infection by a P. multocida strain causing a respiratory tract disease, and the vaccine formulation is prepared to comprise at least one P. multocida PmSLP protein or an immunogenically equivalent portion thereof selected from the group of phylogenetic clusters consisting of PmSLP-1, PmSLP-2, PmSLP-3, and PmSLP-4.2, wherein the selected P. multocida PmSLP protein or an immunogenically equivalent portion thereof belongs to the same phylogenetic cluster as a PmSLP protein present in the infecting P. multocida strain.

In one example embodiment, the food production animal can be a bovine animal susceptible to infection by a P. multocida strain causing BRD, and the vaccine formulation is prepared to comprise at least one P. multocida PmSLP protein or an immunogenically equivalent portion thereof selected from the group of phylogenetic clusters consisting of PmSLP-1, PmSLP2, and PmSLP-4.2, wherein the selected P. multocida PmSLP protein or an immunogenically equivalent portion thereof belongs to the same phylogenetic cluster as a PmSLP protein present in the infecting P. multocida strain.

In one example embodiment, the food production animal can be a bovine animal susceptible to infection by a P. multocida strain causing HS, and the vaccine formulation is prepared to comprise at least one P. multocida PmSLP protein or an immunogenically equivalent portion thereof selected from the group of phylogenetic clusters consisting of PmSLP-3, wherein the selected P. multocida PmSLP protein or an immunogenically equivalent portion thereof belongs to the same phylogenetic cluster as a PmSLP protein present in the infecting P. multocida strain.

In one example embodiment, the food production animal can be a porcine animal susceptible to infection by a P. multocida strain causing porcine atrophic rhinitis (PAR), and the vaccine formulation is prepared to comprise at least one P. multocida PmSLP protein or an immunogenically equivalent portion thereof selected from the group of phylogenetic clusters consisting of PmSLP-2, PmSLP-4.1, and PmSLP-4.2, wherein the selected P. multocida PmSLP protein or an immunogenically equivalent portion thereof belongs to the same phylogenetic cluster as a PmSLP protein present in the infecting P. multocida strain.

In one example embodiment, the food production animal can be a porcine animal susceptible to infection by a P. multocida strain causing pneumonic pasteurellosis, and the vaccine formulation is prepared to comprise at least one P. multocida PmSLP protein or an immunogenically equivalent portion thereof selected from the group of phylogenetic clusters consisting of PmSLP-2, PmSLP-4.1, and PmSLP-4.2, wherein the selected P. multocida PmSLP protein or an immunogenically equivalent portion thereof belongs to the same phylogenetic cluster as a PmSLP protein present in the infecting P. multocida strain.

In one example embodiment, the food production animal can be an avian animal susceptible to infection by a P. multocida strain causing fowl cholera, and the vaccine formulation is prepared to comprise a P. multocida PmSLP protein or an immunogenically equivalent portion thereof selected from the group of phylogenetic clusters consisting of PmSLP-3, and PmSLP-4.2, wherein the selected P. multocida PmSLP protein or an immunogenically equivalent portion thereof belongs to the same phylogenetic cluster as a PmSLP protein present in the infecting P. multocida strain.

It is noted that a P. multocida strain may be isolated from infected food production animals and cultured in order to identify the P. multocida strain, and the sequence of the PmSLP polypeptide present therein may be determined using techniques known to those of ordinary skill in the art, see e.g., Pasteurellaceae: Biology, Genomics and Molecular Aspects. Publisher: Caister Academic Press Edited by: Peter Kuhnert (1) and Henrik Christensen (2). Institute of Veterinary Bacteriology, Vetsuisse Faculty University of Bern, Länggass-Str. 122, 3001 Bern, Switzerland (1) and Department of Veterinary Pathobiology, Faculty of Life Science, Copenhagen University, Dyrlaegevej 88, 1870 Frederiksberg, Denmark (2).

Furthermore, in an example embodiment, in preparing a vaccine formulation, in order to treat an animal infected by a P. multocida strain belonging to a specific serogroup, the vaccine is preferably prepared using a PmSLP polypeptide obtained from a P. multocida strain belonging to the same serogroup. Thus, for example, in preparing a vaccine formulation, preferably, in order to treat an animal infected by a P. multocida strain belonging to serogroup A, B, C, D, E, or F, a vaccine formulation comprising a PmSLP polypeptide obtained from a P. multocida strain from the same serogroup is selected for inclusion in the vaccine. Thus, by way of example, if the animal is infected by a P. multocida strain of serogroup A, the vaccine formulation preferably is prepared by selection and inclusion in the formulation of a PmSLP polypeptide or an immunogenically equivalent portion thereof, obtained from P. multocida strain of serogroup A; and, by way of further example, if the animal is infected by a P. multocida strain belonging to serogroup F, the vaccine formulation preferably is prepared by selection and inclusion in the formulation of a PmSLP polypeptide or an immunogenically equivalent portion thereof, obtained from a P. multocida strain belonging to serogroup F. The strain can be the same strain, or another strain, provided said another strain belongs to the same serogroup.

In further preferred example embodiments, consideration is given to both phylogenetic cluster and serogroup. Thus, preferably, in preparing a vaccine formulation, in order to treat an animal infected by a P. multocida strain containing a PmSLP polypeptide belonging to a specific phylogenetic cluster and a specific serogroup, the vaccine formulation is preferably prepared by selecting and using a PmSLP polypeptide belonging to the same phylogenetic cluster and a strain belonging to the same serogroup as the infectious strain. Thus, referring again to FIG. 2, for example, in preparing a vaccine formulation, preferably, in order to treat an animal infected by a P. multocida strain containing PmSLP protein belonging to phylogenetic cluster PmSLP-1, PmSLP-2, PmSLP-3, PmSLP-4.1, or PmSLP-4.2 and the P. multocida strain belonging to serogroup A, B, D, E, or F, a vaccine formulation comprising a PmSLP polypeptide from the same phylogenetic cluster and obtained from strain of the same serogroup is selected for inclusion in the vaccine. The PmSLP polypeptide can be identical to the PmSLP contained in the infectious strain, or different, provided however, the PmSLP polypeptide belongs to the same phylogenetic cluster and is obtained from a strain of the same serogroup as the infectious strain. Thus, by way of example, if the animal is infected by a P. multocida strain containing a PmSLP polypeptide belonging to phylogenetic cluster PmSLP-2 and the infectious strain belongs to serogroup A, the vaccine formulation preferably is prepared to include a PmSLP polypeptide or an immunogenically equivalent portion thereof, obtained from P. multocida strain containing a PmSLP polypeptide belonging to phylogenetic cluster PmSLP-2 and a strain belonging to serogroup A; if the animal is infected by a P. multocida strain containing a PmSLP polypeptide belonging to phylogenetic cluster PmSLP-3 and the strain belongs to serogroup F, the vaccine formulation preferably is prepared to include a PmSLP polypeptide or an immunogenically equivalent portion thereof, obtained from a P. multocida strain containing a PmSLP polypeptide belonging to phylogenetic cluster PmSLP-3, and a strain belonging to serogroup F. The strain can be the same strain, or another strain, provided said another strain belongs to the same phylogenetic cluster and the same serogroup.

Thus, it will now be clear one embodiment, the vaccine formulation can comprises a P. multocida PmSLP protein or an immunogenically equivalent portion thereof elected from the group of phylogenetic clusters consisting of PmSLP-1, PmSLP-2, PmSLP-3, PmSLP-4.1, and PmSLP-4.2, wherein the selected P. multocida PmSLP protein or an immunogenically equivalent portion thereof belongs to the same phylogenetic cluster as a PmSLP protein present in the infecting P. multocida strain, wherein the selected P. multocida PmSLP protein or an immunogenically equivalent portion thereof further is of a P. multocida strain belonging to a serogroup selected from the group consisting of serogroup A, B, D, E, and F, wherein the serogroup is the same as the serogroup of the infecting P. multocida strain.

Additionally, the PmSLP protein can be a PmSLP protein obtained from a P. multocida strain selected from serotype 1, 2, 3, 4, 5, 6, 7, 8, 9, 10, 11, 12, 13, 14, 15, and 16 P. multocida strains, the food production animal is susceptible to infection by a P. multocida strain of the selected serotype. Thus, in one embodiment, in preparing a vaccine formulation, in order to treat an animal infected by a P. multocida strain containing a PmSLP protein belonging to a specific phylogenetic cluster, and a specific serogroup, and a specific serotype, the vaccine is preferably prepared using a PmSLP polypeptide belonging to the same phylogenetic cluster, the same serogroup, and the same serotype. Thus, for example, when a P. multocida infection causes hemorrhagic septicemia among bovine species, and the strain is a B: 2 strain, the PmSLP protein may be obtained from the B: 2 strain; or, for example, when a P. multocida infection causes progressive atrophic rhinitis among porcine species, and the strain belongs to serogroup D, the PmSLP protein may be obtained from a P. multocida strain belonging to serogroup D, and so on. It is noted that specific P. multocida strains can be obtained (e.g., from a collection of microbial species, such as the American Type Culture Collection (ATCC)) or isolated, for example, from infected food production animals, and the serogroup and/or serotype of a strain, can be determined using methods known to those of skill in the art (see: for example, Wilson, M. et al., 1992, J. Clin. Microbiol, 1518-1524; Arumugam, N., et al. 2011, Tropical Biomed. 28 (1) 55-63).

In a further example embodiment, the food production animal can be selected to be a ruminant species, and a vaccine formulation can be prepared for administration to the ruminant species, wherein the P. multocida infection causes a respiratory tract disease, and wherein the PmSLP protein comprises SEQ. ID NO: 2, SEQ. ID NO: 4, SEQ. ID NO: 6, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 65, SEQ. ID NO: 67, SEQ. ID NO: 69, SEQ. ID NO: 71, SEQ. ID NO: 73, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, or SEQ. ID NO: 91, or an immunogenically equivalent portion thereof, to prevent or ameliorate a P. multocida infection causing the respiratory tract disease.

In a further example embodiment, the food production animal can be a selected to be bovine species, and a vaccine formulation can be prepared for administration to the bovine species, wherein the P. multocida infection causes BRD, and wherein the PmSLP protein comprises SEQ. ID NO: 2, SEQ. ID NO: 4, SEQ. ID NO: 10, SEQ. ID NO: 12, SEQ. ID NO: 14 SEQ. ID NO: 16, SEQ. ID NO: 18, SEQ. ID. NO: 22, SEQ. ID. NO: 26, SEQ. ID. NO: 28, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 65, SEQ. ID NO: 67, SEQ. ID NO: 69, SEQ. ID NO: 71, SEQ. ID NO: 73, SEQ. ID NO: 77, or SEQ. ID NO: 91, or an immunogenically equivalent portion thereof, to prevent or ameliorate a P. multocida infection causing BRD.

In a further example embodiment, the food production animal can be selected to be a bovine species, and a vaccine formulation can be prepared for administration to the bovine species, wherein the P. multocida infection causes HS, and wherein the PmSLP protein comprises SEQ. ID NO: 6, SEQ. ID NO: 20, SEQ. ID NO: 24, SEQ. ID NO: 30, SEQ. ID NO: 32, SEQ. ID NO: 34, SEQ. ID NO: 36, SEQ. ID NO: 53, SEQ. ID NO: 79, SEQ. ID NO: 81, SEQ. ID NO: 83, SEQ. ID NO: 85, SEQ. ID NO: 87, or SEQ. ID NO: 89, or an immunogenically equivalent portion thereof, to prevent or ameliorate a P. multocida infection causing HS.

In a further example embodiment, the food production animal can be selected to be a porcine species, and a vaccine formulation can be prepared for administration to the porcine species, wherein the P. multocida infection causes pneumonic pasteurellosis, and wherein the PmSLP protein comprises SEQ. ID NO: 4, SEQ. ID NO: 8, SEQ. ID NO: 10, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 75, SEQ. ID NO: 77, or SEQ. ID NO: 91, or an immunogenically equivalent portion thereof, to prevent or ameliorate a P. multocida infection causing pneumonic pasteurellosis.

In a further example embodiment, the food production animal can be a porcine species, and a vaccine formulation can be prepared for administration to the porcine species, wherein the P. multocida infection causes PAR, and wherein the PmSLP protein comprises SEQ. ID NO: 4, SEQ. ID NO: 8, SEQ. ID NO: 10, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 75, SEQ. ID NO: 77, or SEQ. ID NO: 91, or an immunogenically equivalent portion thereof, to prevent or ameliorate a P. multocida infection causing PAR.

In yet a further example embodiment, the food production animal can be an avian species, and the vaccine formulation can be prepared for administration to the avian species, wherein the P. multocida infection causes fowl cholera, and wherein the PmSLP protein comprises SEQ. ID NO: 6, SEQ. ID NO: 10, SEQ. ID NO: 20, SEQ. ID NO: 24, SEQ. ID NO: 30, SEQ. ID NO: 32, SEQ. ID NO: 34, SEQ. ID NO: 36, SEQ. ID NO: 40, SEQ. ID NO: 53, SEQ. ID NO: 55, SEQ. ID NO: 61, SEQ. ID NO: 77, SEQ. ID NO: 79, SEQ. ID NO: 81, SEQ. ID NO: 83, SEQ. ID NO: 85, SEQ. ID NO: 87, or SEQ. ID NO: 89, or an immunogenically equivalent portion thereof, to prevent or ameliorate a P. multocida infection causing pneumonic pasteurellosis.

It is noted, however, that the vaccine formulations of the present disclosure can be used to ameliorate or prevent infections by P. multocida strains, other than the strains from which the PmSLP protein included in a veterinary vaccine formulation is selected. Thus, the vaccine formulations of the present disclosure, surprisingly, do not necessarily need to include PmSLPs from a plurality of P. multocida strains in order to be used for the treatment of a food production animal, even if the food production animal can be or has been exposed to, or can be or has been infected by a plurality of P. multocida strains. Thus, for example, a vaccine formulation comprising a PmSLP protein obtained from a P. multocida strain of a first serotype, may be used to treat a food production animal for an infection caused by a P. multocida strain of another serotype. In this respect, the veterinary vaccine formulations, even if they include a single PmSLP or immunogenically equivalent portion thereof, can be said to be cross-protective, and may confer homologous or heterologous protection.

Notwithstanding the foregoing, in some embodiments, vaccine formulations may comprise two or more PmSLP proteins. Thus, in example embodiments, vaccine formulations may comprise PmSLP proteins obtained from two or more P. multocida strains, each belonging to a single serogroup selected from serogroup A, B, D, E, or F, or veterinary vaccine formulations may comprise PmSLP proteins obtained from two P. multocida strains, belonging to two or more serogroups selected from A, B, D, E, or F. In such embodiments, the vaccine can prevent or ameliorate infection by strains of two or more P. multocida strains, and such vaccines can be said to be cross-protective.

In further example embodiments, vaccine formulations may comprise two or more PmSLP proteins belonging to two or more strains, each belonging to a single phylogenetic cluster PmSLP-1, PmSLP-2, PmSLP-3, PmSLP-4.1, and PmSLP-4.2, or veterinary vaccine formulations may comprise PmSLP proteins obtained from two or more P. multocida strains, belonging to two or more phylogenetic clusters PmSLP-1, PmSLP-2, PmSLP-3, PmSLP-4.1, and PmSLP-4.2. In such embodiments, the vaccine can prevent or ameliorate infection by strains of two or more P. multocida strains, and such vaccines can be said to be cross-protective.

In further example embodiments, vaccine formulations may comprise two or more PmSLP proteins obtained from two P. multocida strains, each belonging to a single serotype selected from serotype 1, 2, 3, 4, 5, 6, 7, 8, 9, 10, 11, 12, 13, 14, 15, or 16, or veterinary vaccine formulations may comprise PmSLP proteins obtained from two or more P. multocida strains, belonging to two or more serotypes selected from 1, 2, 3, 4, 5, 6, 7, 8, 9, 10, 11, 12, 13, 14, 15, or 16. In such embodiments, the vaccine can prevent or ameliorate infection by strains of two or more P. multocida strains, and such vaccines can be said to be cross-protective.

In further example embodiments, vaccine formulations may comprise at 2 or least 2, 3 or at least 3, 4 or at least 4, 5 or at least 5 PmSLP proteins protein having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or immunogenically equivalent portions thereof.

Thus, to briefly recap, in accordance with the present disclosure P. multocida PmSLP polypeptides or an immunogenically equivalent portion thereof, may be selected for inclusion in veterinary vaccine formulations to treat food production animals infected by P. multocida. Preferably, the selected P. multocida PmSLP polypeptide belongs to the same phylogenetic cluster as a PmSLP polypeptide contained by the infecting P. multocida strain.

Turning next to the preparation of vaccine formulations, in an aspect hereof, in order to prepare a vaccine formulation, a preparation comprising a PmSLP protein or immunogenically equivalent portion thereof may be combined with at least one other veterinary pharmaceutically acceptable in ingredient, including, but not limited to, a diluent, an excipient, a carrier, an adjuvant, or mixtures thereof, whereby the PmSLP protein or immunogenically equivalent portion thereof and at least one other ingredient are mixed together or blended or homogenized or otherwise prepared until the vaccine formulation is formed.

The amount of PmSLP protein or immunogenically equivalent portion thereof in the veterinary vaccine formulation may vary. In general, consideration is given to the dose to be administered to an animal. Doses for the PmSLP protein or an immunogenically equivalent portion thereof may be formulated to include PmSLP protein or an immunogenically equivalent portion thereof, in quantities ranging from about 1 μg/kg of animal body weight to about 0.25 mg/kg of animal body weight, preferably about 1 μg/kg of animal body weight to about 100 μg/kg of animal body weight. Furthermore, vaccine formulations are preferably formulated so that a dose comprises at least about 0.001% by weight or volume, at least 0.025% or about 0.025%, at least 0.05% or about 0.05%, at least 0.1% or about 0.1%, at least 0.5 or about 0.5%, at least 1% or about 1%, at least 5% or about 5%, at least 10% or about 10%, at least 15% or about 15%, at least 20% or about 20%, or at least 25% or about 25%, by weight of the PmSLP protein, so that the ratio of PmSLP to other vaccine constituents (e.g., adjuvants, diluents, carriers, excipients) of the vaccine formulation by weight or volume is at least 0.001:99.999, 0.025:99.975, 0.05:99, 95, 0.01:99.99, 0.5:99.5, 1:99, 5:95, 15:85, 20:80, or 25:75, respectively, by weight or volume. The exact amount necessary, however, will vary depending on the species, age, and general condition of the recipient animal to be treated, the severity of the condition being treated, the particular preparation delivered, the site of administration, the subject biological species, as well as other factors. In this respect, veterinary vaccine formulations may, in particular, vary with respect to the quantity of PmSLP protein or immunogenically equivalent portion thereof included in a dose, depending on the species of the food production animal to which the vaccine formulation is administered. A suitable effective amount can be readily determined by one of skill in the art. Thus, a therapeutically effective amount of the PmSLP protein or immunogenically equivalent portion to be included in the veterinary vaccine formulations of the present disclosure will be an amount sufficient to bring about amelioration or prevention of disease or condition symptoms and will fall in a relatively broad range that can be determined through routine trials.

Veterinary vaccine formulations comprising the PmSLP protein or immunogenically equivalent portion thereof of the present disclosure preferably further are prepared by combining the PmSLP protein or immunogenically equivalent portion thereof with e.g., carriers, excipients, diluents, and auxiliary substances, such as wetting or emulsifying agents, pH buffering substances and the like. These carriers, excipients, diluents, and auxiliary substances are veterinary pharmaceutically acceptable ingredients. Veterinary pharmaceutically acceptable excipients include, but are not limited to, liquids such as water, saline, polyethyleneglycol, hyaluronic acid, glycerol, and ethanol. Veterinary pharmaceutically acceptable salts can also be included in the formulation, for example, mineral acid salts such as hydrochlorides, phosphates, sulfates, and the like; and the salts of organic acids such as acetates, propionates, benzoates, and the like. It is also preferred, although not required, that the vaccine formulation will contain a veterinary pharmaceutically acceptable carrier that serves as a stabilizer, particularly in order to stabilize the polypeptides of the present disclosure. Examples of suitable carriers that also act as stabilizers for peptides include, without limitation, veterinary pharmaceutical grades of dextrose, sucrose, lactose, sorbitol, inositol, dextran, and the like. Other suitable carriers include, again without limitation, starch, cellulose, sodium or calcium phosphates, citric acid, glycine, polyethylene glycols (PEGs), and combinations thereof. Carriers, excipients, diluents, may constitute, for example, from about 10% to about 95% by weight or volume of the vaccine formulation.

Further, auxiliary agents such as freeze-drying stabilizers, wetting or emulsifying agents, pH buffering agents, gelling or viscosity enhancing additives, and preservatives may also be included in the vaccine formulations of the present disclosure. Vaccine formulations generally comprise less than about 5% by weight of such auxiliary agents.

In order to augment an immune response in an animal, the veterinary vaccine formulations provided herein further preferably include one or more adjuvants, such as pharmacological agents, cytokines, or the like. Suitable adjuvants include any substance that enhances the immune response of the recipient animal to the immunogenic PmSLP protein or immunogenically equivalent portion thereof of the disclosure. Non-limiting examples of adjuvants include cytokines, e.g., IL-1, IL-2, IL-12, IL-6, and further include inorganic salts, e.g., aluminum hydroxide, aluminum phosphate, and calcium phosphate; oil emulsions, e.g., mineral oil, MF59, QS-21, Montanide™ ISA51, Montanide™ ISA61, Montanide™ ISA 61 VG, Montanide™ Gel 02, Montanide™ ISA-720, or Emulsigen D®; Isocoms, e.g., ISCOMATRIX; microbial derivatives, e.g., monophosphoryl lipid A (MPLA), macrophage-activating protein-2, virosomes, LT/CT, CpG; natural polymers, e.g., polysaccharides; and synthetic polymers, e.g., polyanhydrides and polyesters, or nucleic acid analogs, such as Poly I:C. Adjuvants may be administered, for example, as proteins or other macromolecules at the same time (e.g., by inclusion in the vaccine formulation), prior to, or subsequent to, administration of the polypeptide antigens. When included in a vaccine formulation, adjuvants may constitute, for example, from 0.1% or about 0.1% to 50% or about 50%, from 0.1% or about 0.1% to 20% or about 20%, or from 1% or about 1% to 10% or about 10% by weight or volume of a vaccine formulation.

In light of the foregoing, it will be understood that the present disclosure provides, in another aspect, methods for preparing vaccine formulations. In this respect, the present disclosure provides, in one example embodiment, a method for preparing a veterinary vaccine formulation for the prevention or amelioration of P. multocida infection in a food production animal susceptible to P. multocida infection, the method comprising:
(i) diagnosing a P. multocida infection in a food production animal;
(ii) identifying the phylogenetic cluster to a which a PmSLP protein contained in the infecting P. multocida belongs, the phylogenetic cluster being selected from PmSLP-1, PmSLP-2, PmSLP-3, PmSLP-4.1 or PmSLP-4.2; and
(iii) preparing a vaccine formulation comprising a P. multocida PmSLP protein or immunogenically equivalent portion thereof which belongs to the identified phylogenetic cluster together with a veterinary pharmaceutically acceptable adjuvant to form a veterinary vaccine formulation comprising an effective amount of the P. multocida PmSLP protein or the immunogenically equivalent portion thereof to treat a food production animal susceptible to P. multocida infection.

In one embodiment, in an aspect, the method additionally can comprise identifying the serogroup of the infecting P. multocida strain, the serogroup being selected from the group consisting of serogroup A, B, D, E, and F, and the vaccine being prepared using a P. multocida PmSLP protein or an immunogenically equivalent portion thereof from the same or another P. multocida strain belonging to the selected serogroup.

In a further example embodiment, the present disclosure provides a method for preparing a veterinary vaccine formulation comprising a PmSLP protein or an immunogenically equivalent portion thereof, the method comprising:
(a) providing a chimeric nucleic acid sequence comprising as operably linked components:
   (i) a nucleic acid sequence encoding a PmSLP protein or an immunogenically equivalent portion thereof; and
   (ii) one or more nucleic acid sequences capable of controlling expression in a host cell;
(b) introducing the chimeric nucleic acid sequence into a host cell;
(c) growing the host cell to produce the PmSLP protein or the immunogenically equivalent portion thereof; and
(d) recovering the PmSLP protein or the immunogenically equivalent portion thereof; and
(e) formulating the PmSLP protein or the immunogenically equivalent portion thereof together with an adjuvant to form a veterinary vaccine formulation comprising an effective amount of the PmSLP protein or the immunogenically equivalent portion thereof to treat a food production animal susceptible to P. multocida infection.

The vaccine formulations of the present disclosure may be used to prevent infection or disease caused by pathogenic infectious P. multocida in food production animals. The vaccine formulations may be used to immunize any food production animal, including any bovine species, porcine species, or avian species.

The veterinary vaccine formulations of the present may be administered to a food production animal using any convenient administration means. Thus, for example, the veterinary vaccine formulations may be injected, for example, intramuscularly or subcutaneously, or the vaccine formulations may be orally administered to the animal, for example, as a feed supplement. It will be understood that, in this respect, the administration means and techniques, such as, for example, in the case of injections, the gauge of the injection needle, may vary depending on the animal. The dosage of the veterinary vaccine formulation will be dependent upon the disease, the route of administration, the animal species, body weight, and other standard factors. In this respect, a person of ordinary skill in the art can readily titrate the appropriate dosage for an effective amount as well as select a suitable method of administration.

It is further noted that the veterinary vaccine formulations of the present disclosure may be administered prophylactically, i.e., in order to prevent P. multocida infection in a food production animal, or in order to ameliorate symptoms associated with P. multocida infection following the occurrence of an infection in a food production animal.

The administration of the veterinary vaccine formulations of the present disclosure generally elicits an immune response in the subject food production animal. Thus, antibodies against the PmSLP protein included in the vaccine formulation may be formed by the food production animal. In some embodiments, anti-PmSLP antibodies can be detected in the blood serum of the food production animal at least 7 days, at least 2 weeks, at least 5 weeks, at least 10 weeks, at least 13 weeks, at least 26 weeks, or at least 52 weeks following administration of the vaccine formulation.

In light of the foregoing, it will now be understood that, in another aspect, the present disclosure provides, in an example embodiment, a use of PmSLP protein or an immunogenically equivalent portion thereof to prepare a veterinary vaccine formulation comprising the PmSLP protein or an immunogenically equivalent portion thereof together with a veterinary pharmaceutically acceptable adjuvant.

In light of the foregoing, it will now be understood that, in another aspect, the present disclosure provides, in an example embodiment, a use of a veterinary vaccine formulation comprising a PmSLP protein or an immunogenically equivalent portion thereof together with a veterinary pharmaceutically acceptable adjuvant to ameliorate or prevent a P. multocida infection in a food production animal susceptible to P. multocida infection.

As can now be understood, veterinary vaccine formulations comprising a PmSLP protein, or an immunogenically equivalent portion thereof may be prepared. The veterinary vaccine formulations can be administered to a food production animal to ameliorate or prevent infection of the animal by P. multocida.

Of course, the above-described example embodiments of the present disclosure are intended to be illustrative and in no way limiting. The embodiments are susceptible to many modifications or composition, details, and order of operation. The invention and this disclosure are intended to encompass all such modifications within its scope, as defined by the claims, which should be given a broad interpretation consistent with the description as a whole.

### SUMMARY OF SEQUENCES

SEQ. ID NO: 1 and SEQ. ID NO: 2 set forth the polynucleotide sequence and deduced amino acid, respectively, of a mature PmSLP-1 of P. multocida (belonging to phylogenetic cluster 1).

SEQ. ID NO: 3 and SEQ. ID NO: 4 set forth the polynucleotide sequence and deduced amino acid, respectively, of a mature PmSLP-2 of P. multocida (belonging to phylogenetic cluster 2).

SEQ. ID NO: 5 and SEQ. ID NO: 6 set forth the polynucleotide sequence and deduced amino acid, respectively, of a mature PmSLP-3 of P. multocida (belonging to phylogenetic cluster 3).

SEQ. ID NO: 7 and SEQ. ID NO: 8 set forth the polynucleotide sequence and deduced amino acid, respectively, of a mature PmSLP-4 of P. multocida (belonging to phylogenetic cluster 4.1).

SEQ. ID NO: 9 and SEQ. ID NO: 10 set forth the polynucleotide sequence and deduced amino acid, respectively, of a mature PmSLP-4 of P. multocida (belonging to phylogenetic cluster 4.2).

SEQ. ID NO: 11 and SEQ. ID NO: 12 set forth the polynucleotide sequence and deduced amino acid, respectively, of a PmSLP-1 of P. multocida with a 14-residue anchor truncation of PmSLP, a thrombin cleavage site, and a C terminal polyhistidine tag (belonging to phylogenetic cluster 1).

SEQ. ID NO: 13 and SEQ. ID NO: 14 set forth the polynucleotide sequence and deduced amino acid, respectively, of a PmSLP-1 of P. multocida with a 14-residue anchor truncation (belonging to phylogenetic cluster 1).

SEQ. ID NO: 15 and SEQ. ID NO: 16 set forth the polynucleotide sequence and deduced amino acid, respectively, of a PmSLP-1 of P. multocida with a 126-residue anchor truncation, a thrombin cleavage site, and C terminal polyhistidine tag (belonging to phylogenetic cluster 1).

SEQ. ID NO: 17 and SEQ. ID NO: 18 set forth the polynucleotide sequence and deduced amino acid, respectively, of a PmSLP-1 of P. multocida with a 94-residue anchor truncation, introduced SERp1 mutations, a thrombin cleavage site, and C terminal polyhistidine tag (belonging to phylogenetic cluster 1).

SEQ. ID NO: 19 and SEQ. ID NO: 20 set forth the polynucleotide sequence and deduced amino acid, respectively, of a PmSLP-3 with a 15-residue anchor truncation and a mutation of the 16th residue from valine to methionine of P. multocida (belonging to phylogenetic cluster 3).

SEQ. ID NO: 21 and SEQ. ID NO: 22 set forth the polynucleotide sequence and deduced amino acid, respectively, of a PmSLP-1 from P. multocida challenge strain H246 (belonging to phylogenetic cluster 1).

SEQ. ID NO: 23 and SEQ. ID NO: 24 set forth the polynucleotide sequence and deduced amino acid, respectively, of a PmSLP-3 from P. multocida challenge strain H229 (belonging to phylogenetic cluster 3).

SEQ. ID NO: 25 and SEQ. ID NO: 26 set forth the polynucleotide sequence and deduced amino acid, respectively, of a PmSLP-1 with a C-terminal FLAG tag from P. multocida (belonging to phylogenetic cluster 1).

SEQ. ID NO: 27 and SEQ. ID NO: 28 set forth the polynucleotide sequence and deduced amino acid, respectively, of a PmSLP-1 from P. multocida with a 14-residue anchor truncation, a thrombin cleavage site, and C terminal polyhistidine tag from P. multocida (belonging to phylogenetic cluster 1).

SEQ. ID NO: 29 and SEQ. ID NO: 30 set forth the polynucleotide sequence and deduced amino acid, respectively, of a PmSLP-3 from P. multocida with a 15-residue anchor truncation, a mutation of the 16th residue from valine to methionine, a thrombin cleavage site, and C terminal polyhistidine tag from P. multocida (belonging to phylogenetic cluster 3).

SEQ. ID NO: 31 and SEQ. ID NO: 32 set forth the polynucleotide sequence and deduced amino acid, respectively, of a PmSLP-1 from P. multocida (belonging to phylogenetic cluster 1) with a 14-residue anchor truncation as a fusion protein to PmSLP-3 (belonging to phylogenetic cluster 3) with a 15-residue anchor truncation and a mutation of the 16th residue from valine to methionine. This construct lacks a signal peptide and contains a thrombin cleavage site between the two PmSLP proteins, as well as a second thrombin cleavage site on the C terminus, followed by a polyhistidine tag.

SEQ. ID NO: 33 and SEQ. ID NO: 34 set forth the polynucleotide sequence and deduced amino acid, respectively, of a PmSLP-1 from P. multocida (belonging to phylogenetic cluster 1) with a 14-residue anchor truncation as a fusion protein to PmSLP-3 (belonging to phylogenetic cluster 3) with a 15-residue anchor truncation and a mutation of the 16th residue from valine to methionine. This construct lacks a signal peptide, contains a serine-alanine linker peptide between the two PmSLP proteins, a C terminal thrombin cleavage site, followed by a polyhistidine tag.

SEQ. ID NO: 35 and SEQ. ID NO: 36 set forth the polynucleotide sequence and deduced amino acid, respectively, of a full length PmSLP-1 protein from P. multocida belonging to phylogenetic cluster 1), including the endogenous signal peptide as a fusion protein to PmSLP-3 (belonging to phylogenetic cluster 3) with a 15-residue anchor truncation and a mutation of the 16th residue from valine to methionine. This construct contains a serine-alanine linker peptide between the two PmSLP proteins, a C terminal thrombin cleavage site, followed by a polyhistidine tag.

SEQ. ID NO: 37 and SEQ. ID NO: 38 set forth the polynucleotide sequence and deduced amino acid, respectively, of a mature PmSLP from P. multocida (belonging to phylogenetic cluster 1) from strain 1500E.

SEQ. ID NO: 39 and SEQ. ID NO: 40 set forth the polynucleotide sequence and deduced amino acid, respectively, of a PmSLP-4 from P. multocida (belonging to phylogenetic cluster 4.2) from strain HS-Canada1.

SEQ. ID NO: 41 sets forth a thrombin cleavage site contained once within SEQ. ID NO: 12, SEQ. ID NO: 16, SEQ. ID NO: 18, SEQ. ID NO: 28, SEQ. ID NO: 30, SEQ. ID NO: 34, SEQ. ID NO: 36, SEQ. ID NO: 51, SEQ. ID NO: 53, SEQ. ID NO: 55, SEQ. ID NO: 57, and contained twice within SEQ. ID NO: 32.

SEQ. ID NO: 42 sets forth the endogenous signal sequence contained within SEQ. ID NO: 36.

SEQ. ID NO: 43 sets forth a polypeptide portion contained within SEQ. ID NO: 34 and SEQ. ID NO: 36 comprising a serine-alanine (SA) linker.

SEQ. ID NO: 44 sets forth a first nucleic acid primer sequence.

SEQ. ID NO: 45 sets forth a second nucleic acid primer sequence.

SEQ. ID NO: 46 sets forth a third nucleic acid primer sequence.

SEQ. ID NO: 47 sets forth a fourth nucleic acid primer sequence.

SEQ. ID NO: 48 sets forth a fifth nucleic acid primer sequence.

SEQ. ID NO: 49 sets forth a sixth nucleic acid primer sequence.

SEQ. ID NO: 50 and SEQ. ID NO 51 set forth the polynucleotide sequence and deduced amino acid, respectively, of a PmSLP-1 from P. multocida (belonging to phylogenetic cluster 1) with a 14-residue anchor truncation as a fusion protein to PmSLP-2 (belonging to phylogenetic cluster 2) with a 14-residue anchor truncation and a mutation of the 15^{th} residue from lysine to methionine. This construct lacks a signal peptide, contains a serine-alanine linker peptide between the two PmSLP proteins, a C terminal thrombin cleavage site, followed by a polyhistidine tag.

SEQ. ID NO: 52 and SEQ. ID NO: 53 set forth the polynucleotide sequence and deduced amino acid, respectively, of a PmSLP-1 from P. multocida (belonging to phylogenetic cluster 1) with a 14-residue anchor truncation as a fusion protein to PmSLP-2 (belonging to phylogenetic cluster 2) with a 14-residue anchor truncation and a mutation of the 15^{th} residue from lysine to methionine, as a fusion protein to the PmSLP-3 (belonging to phylogenetic cluster 3) with a 15-residue anchor truncation and a mutation of the 16^{th} residue from valine to methionine. This construct contains a serine-alanine linker peptide between each of the three PmSLP proteins, a C terminal thrombin cleavage site, followed by a polyhistidine tag.

SEQ. ID NO: 54 and SEQ. ID NO: 55 set forth the polynucleotide sequence and deduced amino acid, respectively, of a PmSLP-1 from P. multocida (belonging to phylogenetic cluster 1) with a 14-residue anchor truncation as a fusion protein to PmSLP-2 (belonging to phylogenetic cluster 2) with a 14-residue anchor truncation and a mutation of the 15^{th} residue from lysine to methionine, as a fusion protein to the PmSLP-4 (belonging to phylogenetic cluster 4.2) with a 41-residue N-terminal truncation and a mutation of the 42^{nd} residue from alanine to methionine. This construct contains a serine-alanine linker peptide between each PmSLP protein, a C terminal thrombin cleavage site, followed by a polyhistidine tag.

SEQ. ID NO: 56 and SEQ. ID NO: 57 set forth the polynucleotide sequence and deduced amino acid, respectively, of a PmSLP-2 from P. multocida (belonging to phylogenetic cluster 2) of P. multocida with a 14-residue anchor truncation and a mutation of the 15^{th} residue from lysine to methionine. This construct contains a C terminal thrombin cleavage site followed by a polyhistidine tag.

SEQ. ID NO: 58 and SEQ. ID NO: 59 set forth the polynucleotide sequence and deduced amino acid, respectively, of a PmSLP-2 from P. multocida (belonging to phylogenetic cluster 2) with a 14-residue anchor truncation and a mutation of the 15^{th} residue from lysine to methionine.

SEQ. ID NO: 60 and SEQ. ID NO: 61 set forth the polynucleotide sequence and deduced amino acid, respectively, of a PmSLP-4 from P. multocida (belonging to phylogenetic cluster 4.2) with a 15-residue anchor truncation.

SEQ. ID NO: 62 and SEQ. ID NO: 63 set forth the polynucleotide sequence and deduced amino acid, respectively, of a PmSLP-4 from P. multocida (belonging to phylogenetic cluster 4.1 with a 15-residue anchor truncation.

SEQ. ID NO: 64 and SEQ. ID NO: 65 set forth the polynucleotide sequence and deduced amino acid, respectively, of a PmSLP-1 from P. multocida (belonging to phylogenetic cluster 1) with a 14-residue anchor truncation, a mutation of the 214^{th} residue from valine to aspartic acid, a C terminal thrombin cleavage site, followed by a polyhistidine tag.

SEQ. ID NO: 66 and SEQ. ID NO: 67 set forth the polynucleotide sequence and deduced amino acid, respectively, of a PmSLP-1 from P. multocida (belonging to phylogenetic cluster 1) with a 14-residue anchor truncation, a mutation of the 240^{th} residue from glutamic acid to alanine, a C terminal thrombin cleavage site, followed by a polyhistidine tag.

SEQ. ID NO: 68 and SEQ. ID NO: 69 set forth the polynucleotide sequence and deduced amino acid, respectively, of a mature PmSLP-4 from P. multocida (belonging to an unclassified phylogenetic cluster).

SEQ. ID NO: 70 and SEQ. ID NO: 71 set forth the polynucleotide sequence and deduced amino acid, respectively, of a mature PmSLP-1 from P. multocida (belonging to phylogenetic cluster 1).

SEQ. ID NO: 72 and SEQ. ID NO: 73 set forth the polynucleotide sequence and deduced amino acid, respectively, of a mature PmSLP-1 from P. multocida (belonging to phylogenetic cluster 1).

SEQ. ID NO: 74 and SEQ. ID NO: 75 set forth the polynucleotide sequence and deduced amino acid, respectively, of a mature PmSLP-4 from P. multocida (belonging to phylogenetic cluster 4.1).

SEQ. ID NO: 76 and SEQ. ID NO: 77 set forth the polynucleotide sequence and deduced amino acid, respectively, of a mature PmSLP-4 from P. multocida (belonging to phylogenetic cluster 4.2).

SEQ. ID NO: 78 and SEQ. ID NO: 79 set forth the polynucleotide sequence and deduced amino acid, respectively, of a mature PmSLP-3 from P. multocida (belonging to phylogenetic cluster 3).

SEQ. ID NO: 80 and SEQ. ID NO: 81 set forth the polynucleotide sequence and deduced amino acid, respectively, of a mature PmSLP-3 from P. multocida (belonging to phylogenetic cluster 3).

SEQ. ID NO: 82 and SEQ. ID NO: 83 set forth the polynucleotide sequence and deduced amino acid, respectively, of a mature PmSLP-3 from P. multocida (belonging to phylogenetic cluster 3).

SEQ. ID NO: 84 and SEQ. ID NO: 85 set forth the polynucleotide sequence and deduced amino acid, respectively, of a mature PmSLP-3 from P. multocida (belonging to phylogenetic cluster 3).

SEQ. ID NO: 86 and SEQ. ID NO: 87 set forth the polynucleotide sequence and deduced amino acid, respectively, of a mature PmSLP-3 from P. multocida (belonging to phylogenetic cluster 3).

SEQ. ID NO: 88 and SEQ. ID NO: 89 set forth the polynucleotide sequence and deduced amino acid, respectively, of a mature PmSLP-3 from P. multocida (belonging to phylogenetic cluster 3).

SEQ. ID NO: 90 and SEQ. ID NO: 91 set forth the polynucleotide sequence and deduced amino acid, respectively, of a mature PmSLP-2 from P. multocida (belonging to phylogenetic cluster 2).

SEQ. ID NO: 92 and SEQ. ID NO: 93 set forth the polynucleotide sequence and deduced amino acid, respectively, of a PmSLP-1 from P. multocida (belonging to phylogenetic cluster 1).

SEQ. ID NO: 94 and SEQ. ID NO: 95 set forth the polynucleotide sequence and deduced amino acid, respectively, of a mature PmSLP-2 from P. multocida (belonging to phylogenetic cluster 2).

SEQ. ID NO: 96 and SEQ. ID NO: 97 set forth the polynucleotide sequence and deduced amino acid, respectively, of a mature PmSLP-4 from P. multocida (belonging to phylogenetic cluster 4.2).

Hereinafter are provided examples of specific implementations for performing the methods of the present disclosure, as well as implementations representing the compositions of the present disclosure. The examples are provided for illustrative purposes only and are not intended to limit the scope of the present disclosure in any way.

### EXAMPLES

### Example 1—Genetic Cloning, Protein Production, and Purification of PmSLP

This Example illustrates a general workflow involved in the production of recombinant PmSLP proteins in E. coli.

### Cloning and Site Directed Mutagenesis

Amino-terminal truncations of PmSLP (SEQ. ID NO: 12) for protein expression and purification were designed based on key structural residues as predicted by XtalPred (Slabinski et al., 2007) and synthesized by restriction-free cloning (van den Ent & Löwe, 2006). All primers were synthesized from Sigma Aldrich and Taq DNA polymerase (Thermo Fisher Scientific) was used in PCR reactions in conditions as recommended by the manufacturers. Forward primers (15 amino acid truncation amplified with primer (SEQ. ID NO: 44), 95 amino acid truncation amplified with primer (SEQ. ID NO: 45), and 121 amino acid truncation amplified with primer (SEQ. ID NO: 46) with complementarity to the truncated amino-terminus and reverse primers (reverse primer (SEQ. ID NO: 47) to the vector plasmid pET52b were used to amplify megaprimers from pET52b containing PmSLP with a carboxy-terminal thrombin cleavage site and decahistidine tag. Megaprimers were subsequently purified by gel electrophoresis and excision following manufacturer instructions (Geneaid). Purified gene fragments were used in a linear amplification reaction around empty pET52b plasmids template. The reaction was incubated with 1U Dpn1 (Thermo Fisher Scientific) at 37° C. for 1 hr to degrade the methylated template. 5 μL of the reaction was used to transform E. coli MM294 cells by heat shock and 1 hr recovery in LB at 37° C. shaking. Transformants were selected on LB agar with 100 μg/mL ampicillin and grown overnight at 37° C. Single colonies were used to inoculate 5 mL LB cultures and were incubated at 37° C. overnight shaking. Plasmids were subsequently purified using MiniPrep kit following manufacturer instructions (Geneaid). Charged residues to mutate in PmSLP were predicted by the UCLA MBI SERp server (Goldschmidt, Cooper, Derewenda, & Eisenberg, 2007). Megaprimers with the mutations were amplified using forward primers (SEQ. ID NO: 48) with ≥10 nt complementarity flanking the mutation and reverse primers (SEQ. ID NO: 49) to the vector. Secondary PCR was performed with purified megaprimers and construct-containing pET52b templates. Constructs were transformed into and propagated in E. coli MM294 and were extracted by MiniPrep kit, as described above. Mutagenesis was confirmed by Sanger sequencing (TCAG) with T7 forward and reverse primers. The SERp1 mutant form of PmSLP-194 is presented in SEQ. ID NO: 17.

### Expression and Purification of Recombinant PmSLP

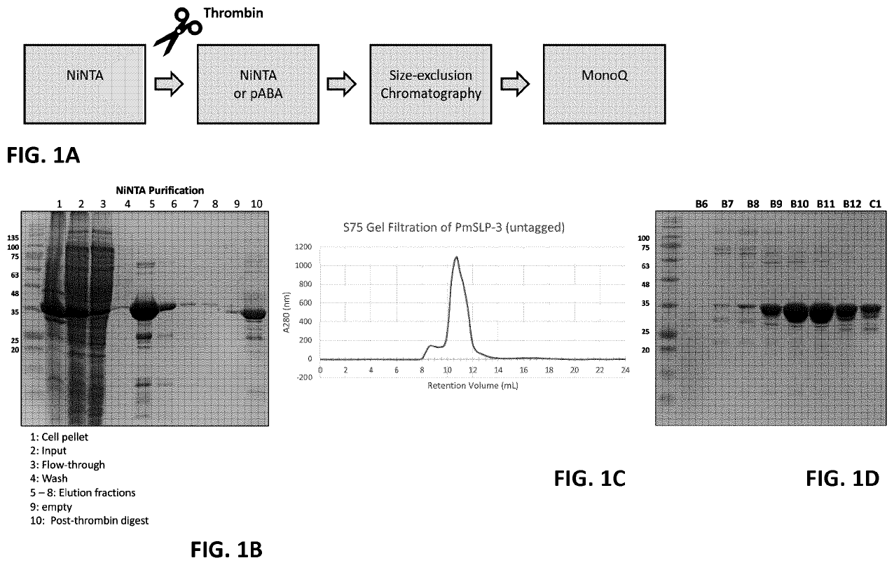

Plasmids containing the PmSLP construct were transformed into E. coli T7 express via 45 second heat shock and 1 hr recovery in in LB at 37° C. shaking. Transformants were selected on LB agar with 100 μg/mL ampicillin. Multiple colonies were used to inoculate starter cultures in 20 mL LB with 100 μg/mL ampicillin and grown at 37° C. shaking for 16 hrs. These overnight cultures were centrifuged at 4,500×g for 4 min and the pelleted cells were used to inoculate 2 L of LB and this larger culture was grown at 37° C. while shaking for approximately 3 hrs or until OD600=0.5. Protein expression was induced with the addition of Isopropyl β-d-1-thiogalactopyranoside (IPTG) to a final concentration of 5 mM and cells continued to grow overnight, shaking at 20° C. Cells were pelleted at 4500×g and resuspended in 40 mL of lysis buffer (50 mM Tris-HCl [pH 8.0], 300 mM NaCl) with 10 mM imidazole, 1 mM phenylmethylsulfonyl fluoride (PMSF), 1 mM benzamidine, 1 mg/ml lysozyme, 0.03 mg/mL DNase I. Cells are lysed by sonication (Branson) for 2.5 minutes and centrifuged for 45 min at 30,000×g to remove cell debris. The supernatant was passed through a 0.45 μm syringe filter and was incubated at 4° C. overnight shaking with 2 mL HisPur Ni-NTA resin (ThermoFisher Scientific). Beads were pelleted for 2 min at 700×g, loaded onto a gravity flow column (Econo-Pac® Bio Rad), and washed with 100 mL cold wash buffer (lysis buffer with 20 mM imidazole). Protein was eluted in 12 mL cold elution buffer (lysis buffer with 400 mM imidazole) and incubated with 2U bovine thrombin (Sigma Aldrich, cat #T4648) in dialysis against 500 mL of 25 mM Tris-HCl [pH 8.0] and 100 mM NaCl at 4° C. overnight. Dialysed sample was incubated with 100 μL HisPur Ni-NTA resin (Thermo Fisher Scientific) and 100 μL p-aminobenamidine-agarose (Sigma Aldrich) for 1 hr shaking at 22° C. Cleaved proteins were 0.22 μm syringe filtered, concentrated to 20 mg/mL by 10K MWCO concentrator (Thermo Fisher Scientific), and purified by size exclusion chromatography (Superdex 75 10/300 GL, GE Healthcare). For antigen studies, PmSLP protein was further purified on a strong anion exchange chromatography column (MonoQ 5/50 GL, GE Healthcare) to remove endotoxins (for workflow, see: FIG. 1A).

Selenomethionine incorporated PmSLP was expressed and purified as above, with a notable difference in the growth medium. The starter culture was inoculated in 50 mL of 0.02 mg/ml methionine supplemented minimal media (M9 with final 0.2% glucose, 1 mM MgSO₄, 1 mM thiamin, 0.02 mg/mL essential L-amino acids) with 100 μg/mL ampicillin, which was pelleted and subcultured into 2 L of minimal media supplemented with 0.02 mg/ml of selenomethionine (ACROS, Thermo Fisher Scientific).

FIG. 1 shows a production and purification workflow for PmSLP proteins along with example gels of purified PmSLP-3. In particular, FIG. 1A depicts is flowchart showing a schematic workflow for the purification of recombinant PmSLP. FIG. 1B depicts a SDS PAGE gel in which samples collected at different stages of Nickel NTA purification of his-tagged PmSLP-3 (SEQ. ID NO: 12) from E. coli lysate are shown (lane 1: whole cell lysate; lane 2: cell pellet; lane 3: soluble fraction; lane 4 flow through fraction; lane 5: wash fraction; lane 6: elution fraction). FIG. 1C shows a S75 gel filtration chromatogram of size exclusion chromatography after removal of the poly-histidine tag of PmSLP-3 (SEQ. ID NO: 14). FIG. 1D shows an SDS-PAGE gel of different fractions (B9, B10, B11, B12, C1, C2) of purified PmSLP-3 after the performance of a polishing step with the MonoQ column. PmSLP-3 migrates within the gel at approximately 35 kDa after tag removal.

### Example 2—Phylogenetic Analysis of PmSLP Clusters and Associated Disease Types

This Example illustrates the PmSLP sequence variability in P. multocida isolates from bovine respiratory disease, bovine hemorrhagic septicemia, swine pasteurellosis and fowl cholera infections in birds, as well as from other host species, by constructing phylogenetic tree based on multiple PmSLP sequences. By evaluation of the phylogenetic tree, PmSLP polypeptides or immunogenic fragments, can be selected for inclusion in a veterinary vaccine formulation.

pmSLP genes were obtained from 263 publicly available assembled P. multocida genomes obtained from public repositories on Jun. 24, 2020 that originated from China (n=45), the USA (n=56), Iran (n=1), India (n=9), the UK (n=6), Thailand (n=3), Pakistan (n=9), Bangladesh (n=1), Canada (n=1), France (n=16), Sri Lanka (n=1), Kazakhstan (n=3), and Myanmar (n=1). Where available, information about what host animal the P. multocida strain was obtained from was included, including cattle (n=87), bison/buffalo (n=17), alpaca (n=2), chicken (n=10), duck (n=11), turkey (n=10), dog (n=1), human (n=12), sheep (n=4), goat (n=2), rabbit (n=20), rodent (n=2), wolf (n=2), cat (n=2), horse (n=2), boar (n=2), pig (n=59), and goose/Anatidae/avian (n=8). For sequences originating from bovine samples, sequences were stratified by disease where available (BRD vs HS) as well as by capsular serotype where annotated.

PmSLP protein sequences were aligned with MAFFT (v7.450) using the G-INS-I algorithm. ProtTest (v3.4.2) was used to identify the most appropriate model of evolution, which was found to be WAG+I+G+F. A phylogenetic tree was generated using PhyML (v3.3.20190909) and is shown in FIG. 2.

Referring to FIG. 2, the phylogenetic tree of PmSLP variants can be seen to separate into five discrete clusters (PmSLP-1, PmSLP-2, PmSLP-3, and PmSLP-4.1, and PmSLP-4.2). On the phylogenetic tree, variants originating from bovine species are shown as the larger dark circles. Furthermore, indicated in the four ring structures in FIG. 2, are from inner to outer ring structure: (1) host species, (2) disease, (3) geographical region, and (4) capsule type. As can be seen, BRD-PmSLP variants represent two of the four major clusters (PmSLP-1 and PmSLP-2) while HS-PmSLP variants represent a single cluster (PmSLP-3). BRD-PmSLP variants were predominantly isolated from samples originating from North America or Europe, while HS-PmSLP variants were primarily isolated from samples originating from Asia and Africa. PmSLP sequences for each cluster include PmSLP-1 (e.g., SEQ. ID NO: 2) from cluster PmSLP-1, PmSLP-2 (e.g., SEQ. ID NO: 4) from cluster PmSLP-2, PmSLP-3 (e.g., SEQ. ID NO: 6) from cluster PmSLP-3, PmSLP-4.1 (e.g., SEQ. ID NO: 8) from cluster PmSLP-4.1, and PmSLP-4.2 (e.g., SEQ. ID NO: 10) from cluster PmSLP-4.2. PmSLP-1 is at minimum 99.7% identical to the cluster PmSLP-1 sequences. PmSLP-2 is at minimum 99.7% identical to the cluster PmSLP-2 sequences and on average 100.0% identical to the cluster PmSLP-2 sequences. PmSLP-3 is at minimum 93.9% identical to cluster PmSLP-3 sequences and on average 99.4% identical to the cluster PmSLP-3 sequences. PmSLP-4.1 is at minimum 99.7% identical to the cluster PmSLP-4.1 sequences and on average 99.8% identical to the cluster PmSLP-4.1 sequences. PmSLP-4.2 is at minimum 96.8% identical to the cluster PmSLP-4.2 sequences and on average 97.2% identical to the cluster PmSLP-4.2 sequences.

### Example 3—Evaluation of IgG Responses and Duration of Immunogenicity Using Various PmSLP-1 Containing Vaccine Formulations in Mice

This Example demonstrates that PmSLP proteins are immunogenic in a mammalian host and can be formulated with various types of vaccine adjuvants to elicit robust, long term antibody responses.

Vaccine formulations consisting of purified PmSLP-1 protein (SEQ. ID NO: 14) formulated with various adjuvants were administered to 4-6-week-old C57BL/6 mice (Charles River) to assess immunogenicity. Each formulation had a total volume of 100 μL/dose and consisted of 20 μg of PmSLP-1 protein formulated with (i) 20% (v/v) Montanide™ Gel 02 (Seppic)+3 μg Poly(I:C) (Invivogen), (ii) 20% (v/v) Montanide™ Gel 02 (Seppic), (iii) 100 μg Aluminum Hydroxide (Invivogen), (iv) 20% (v/v) Emulsigen D® (MVP Adjuvants), (v) 50% (v/v) Montanide™ ISA61 (Seppic). Two doses of each vaccine were administered via sub-cutaneous injection, 21 days apart. Serum samples were collected 20 days post Dose 1, 2 weeks post Dose 2, and at regular intervals for up to 26 weeks post Dose 2.

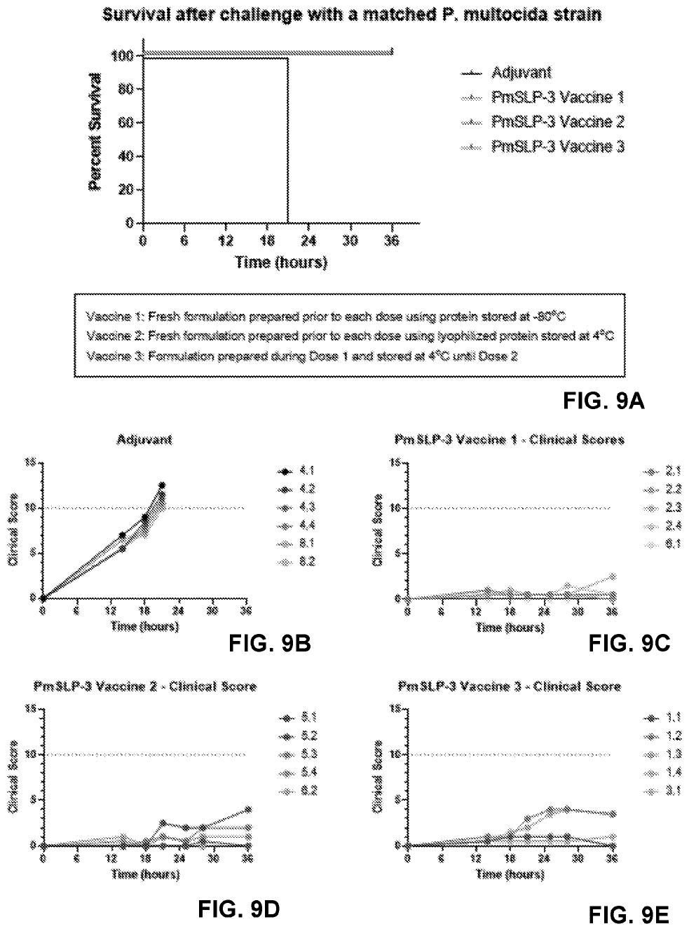

Serum antibody titre against PmSLP-1 protein was measured using ELISA (enzyme linked immunosorbent assay). Total anti-PmSLP-1 IgG, as well as anti-PmSLP-1 IgG subclasses (IgG1, IgG2b, IgG2c) were measured in samples collected after administering one dose and two doses of vaccine (FIG. 9). All five vaccine formulations were able to elicit PmSLP-1 specific IgG in serum after just one dose, and the IgG titre was further augmented after the second dose. PmSLP-1 specific IgG was not detected in unvaccinated naïve control mice. This suggests that PmSLP-1 antigen is immunogenic in vivo and can be used in conjunction with a wide variety of adjuvants. Antibody subclass analysis showed that the predominant subclass elicited in mice is IgG1, followed by IgG2b, while IgG2c antibodies were only consistently elicited in groups that received vaccines containing Montanide™ Gel02+Poly (I:C) or Montanide™ ISA61 adjuvants. Since different IgG subclasses have different effector functions, this suggests that the type of immune response elicited by PmSLP-1 vaccines can be further regulated through adjuvant selection.

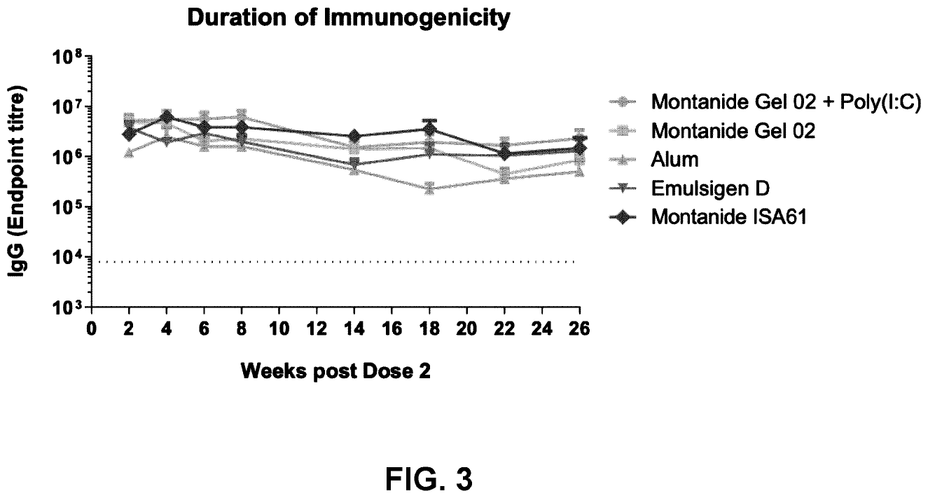

The duration of immunogenicity after administering two doses of the five different PmSLP-1 formulations was assessed for up to 26 weeks (FIG. 3). PmSLP-1 specific IgG titre in serum had remained stable over the duration assessed for all groups, without any significant signs of waning. This suggests that long-lasting antibody responses can be elicited by a PmSLP-1 based vaccine.

### Example 4—Evaluation of PmSLP-1 Efficacy in a Mouse Model of Invasive Infection Against an Antigen-Matched Bovine P. multocida Isolate

This Example illustrates the efficacy of a vaccine formulation comprising PmSLP-1. The vaccine formulation was used to immunize and then challenge mice with a P. multocida isolate that harbours a different PmSLP gene but from the same phylogenetic cluster as the PmSLP-1 protein included in the vaccine. In this Example, a bovine respiratory disease (BRD) P. multocida isolate was used for the challenge. This example further illustrates vaccine formulations can be prepared that are cross-protective against P. multocida infections by different strains.

PmSLP-1 (SEQ. ID NO: 14) was formulated as a vaccine using 20 μg of protein mixed with Montanide™ Gel 02 (Seppic) combined with Poly (I:C) (Invivogen) to a final volume of 100 μl per dose. 4-6-week-old male C57BI/6 mice (Charles River) were given two doses of vaccine at three-week intervals via sub-cutaneous injection. Pre-challenge bleeds were collected prior to infection.

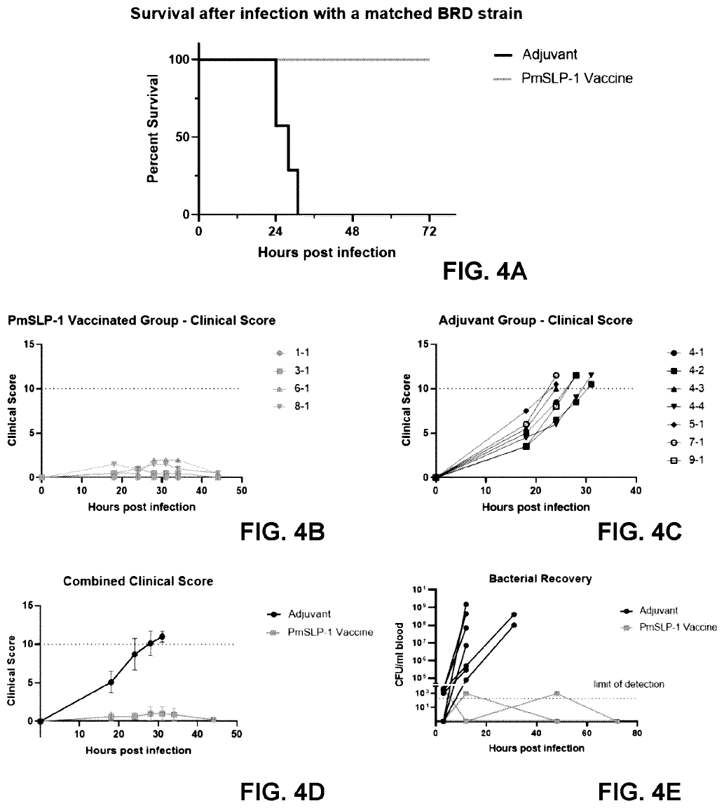

Two weeks after the second dose, anaesthetized animals were challenged via intra-peritoneal injection with approximately 10⁴ CFU of log-phase P. multocida strain H246 (antigen-matched BRD isolate, the confirmed sequence of the PmSLP in this strain is defined in SEQ. ID NO: 22. Strain H246 is a serogroup A strain.). It is noted that SEQ. ID NO: 14 and SEQ. ID NO: 22 exhibit a sequence identity of 100% over the portion of the antigen contained in the immunizing protein. Mice were monitored every 6-12 hours post infection for clinical symptoms including lethargy, breathing, movement, dehydration, diarrhea, posture, and weight loss. A combined clinical score of 10 or higher was considered as clinical endpoint, at which point mice were humanely euthanized (FIGS. 4B, 4C and 4D). Tail vein bleeds were taken at 3, 24, 48, and 72 hours post infection, or at clinical endpoint, and bacteria was enumerated by plating on selective media (FIG. 4E). All mice that received the PmSLP-1 vaccine survived the infection, compared to all animals that received only adjuvant which reached endpoint between 24 and 32 hours post infection (FIG. 4A). Mice that were immunized with adjuvant alone were highly bacteremic, whereas mice that received PmSLP-1 either never had detectable bacteremia or were able to clear any bacteremia by 72 hours post infection.

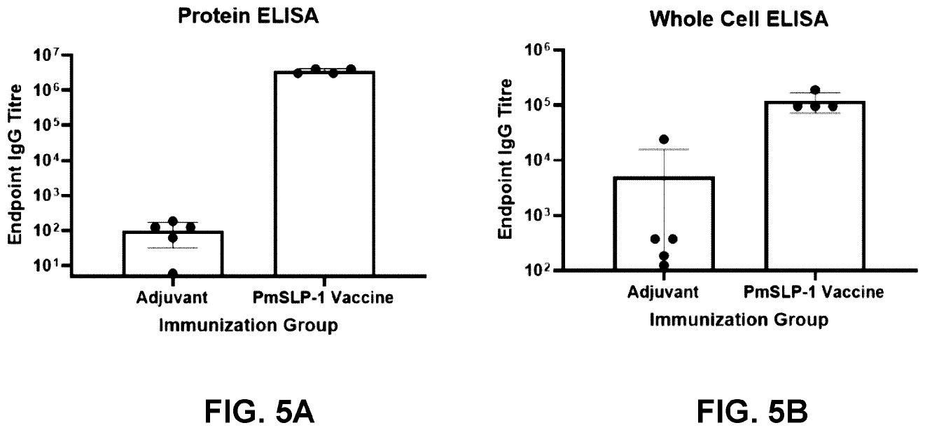

Pre-challenge serum samples were evaluated against purified PmSLP-1 (FIG. 5A) or against heat killed whole P. multocida strain H246 (FIG. 5B). All mice that received PmSLP-1 vaccine had high levels of anti-PmSLP-1 and anti-P. multocida serum IgG prior to the challenge, while mice that received only adjuvant had low levels of serum IgG against either the protein antigen or the whole bacteria.

These results suggest that purified PmSLP-1 protein is a highly effective vaccine antigen against P. multocida strains that harbour the PmSLP gene from the same phylogenetic cluster.

### Example 5—PmSLP-3 Protection in an Acute Mouse Infection Model Against an Antigen-Matched Porcine P. multocida Isolate

This Example illustrates the efficacy of a vaccine formulation comprising PmSLP-3. The vaccine formulation was used to immunize and then challenge mice with a P. multocida isolate that harbours a different PmSLP gene but from the same phylogenetic cluster as the PmSLP-3 protein included in the vaccine. In this Example, a porcine P. multocida isolate was used for the challenge. This example further illustrates vaccine formulations can be prepared that are cross-protective against P. multocida infections by different strains. PmSLP-3 (SEQ. ID NO: 20) was formulated as a vaccine using 20 μg of protein mixed with Montanide™ Gel 02 (Seppic) combined with Poly (I:C) (Invivogen) to a final volume of 100 μl per dose. 4-6-week-old male C57BI/6 mice (Charles River) were given two doses of vaccine at three-week intervals via sub-cutaneous injection. Pre-challenge bleeds were collected prior to infection.

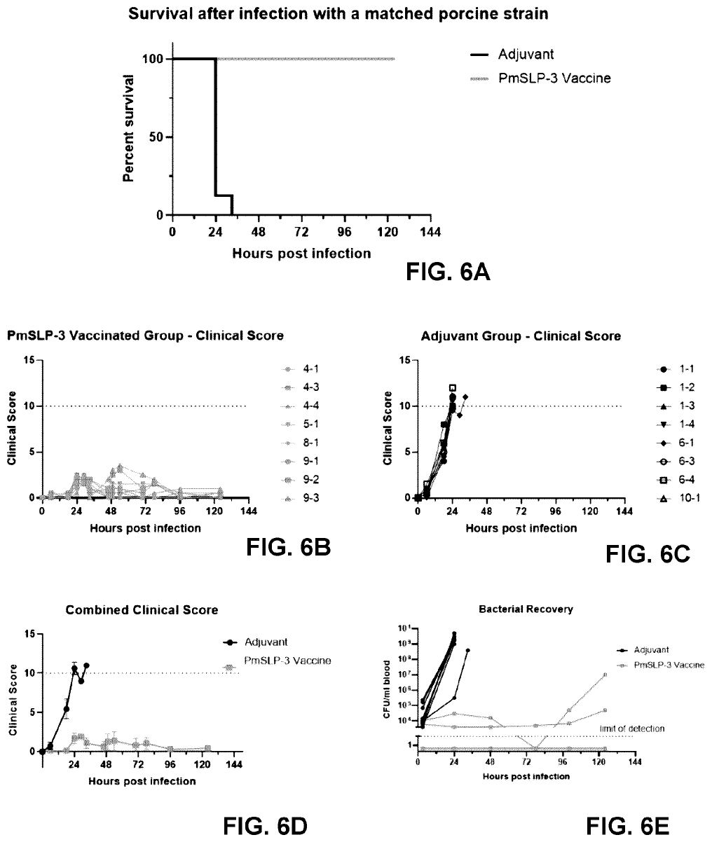

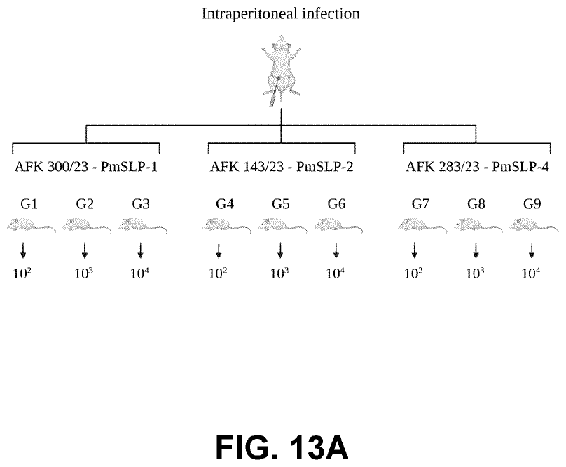

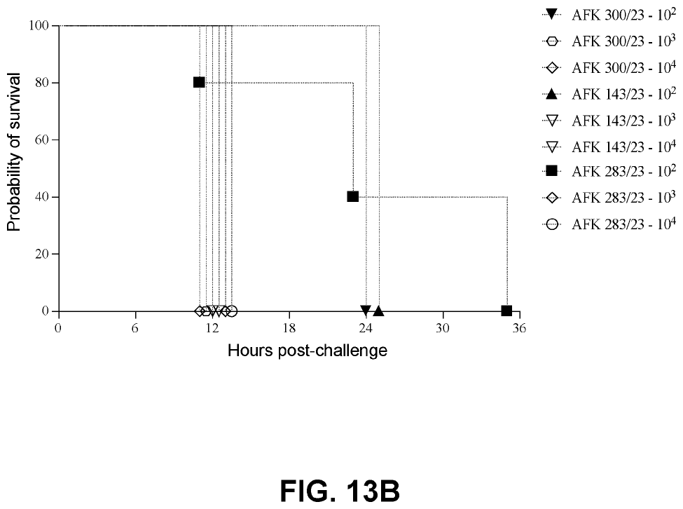

Two weeks after the second dose, anaesthetized animals were challenged via intra-peritoneal injection with approximately 10⁴ CFU of log-phase P. multocida strain H229 (antigen-matched porcine isolate, the confirmed sequence of the PmSLP in this strain is defined in SEQ. ID NO: 24. H229 is a serogroup A strain). It is noted that SEQ. ID NO: 20 and SEQ. ID NO: 24 exhibit a sequence identity of 99.4% over the portion of the antigen contained in the immunizing protein. Mice were monitored every 6-12 hours post infection for clinical symptoms including lethargy, breathing, movement, dehydration, diarrhea, posture, and weight loss. A combined clinical score of 10 or higher was considered as clinical endpoint, at which point mice were humanely euthanized (FIGS. 6B, 6C, and 6D). Tail vein bleeds were taken at 3, 24, 48, 78, 100 and 124 hours post infection, or at clinical endpoint, and bacteria was enumerated by plating on selective media (FIG. 13E). All mice that received PmSLP-3 vaccine survived the infection, compared to all animals that received only adjuvant which reached endpoint between 24 and 32 hours post infection (FIG. 6A). Mice that were immunized with adjuvant alone were highly bacteremic, whereas mice that received PmSLP-3 vaccine either never had detectable bacteremia, or only subclinical bacteremia.

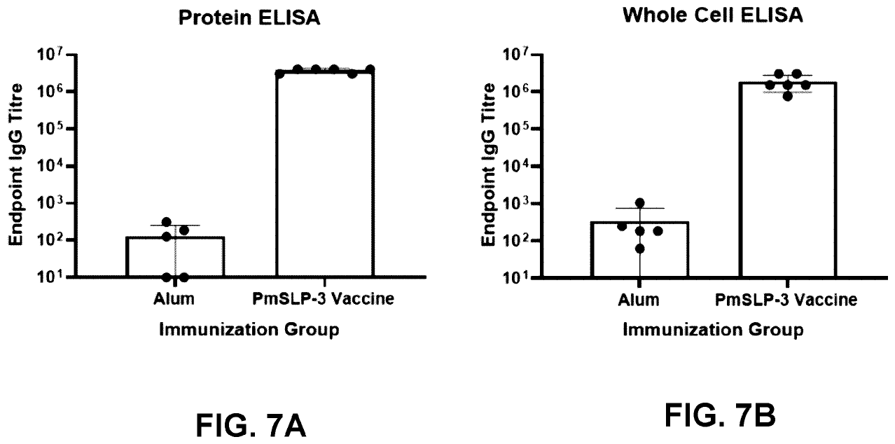

Pre-challenge serum samples were evaluated against purified PmSLP-3 (FIG. 7A) or against heat killed whole P. multocida strain H229 (FIG. 7B). All mice that received PmSLP-3 vaccine had high levels of anti-PmSLP-3 and anti-P. multocida serum IgG prior to challenge, while mice that received only adjuvant had low levels of serum IgG against either the protein antigen or the whole bacteria.

These results suggest that purified PmSLP-3 protein is a highly effective vaccine antigen against P. multocida strains that harbour the PmSLP gene from the same phylogenetic cluster.

### Example 6—Stability and Efficacy of PmSLP Antigens Under Different Storage Conditions

This Example takes into consideration practical aspects of utilizing PmSLPs as vaccine antigens and examines the effect of different storage conditions and lyophilization of the protein. Stability and efficacy of PmSLP antigens were evaluated by using both biophysical methods as well as in protection studies in a mouse model.

For the biophysical approach, stability of purified PmSLP-3 protein (SEQ. ID NO: 20) stored for one year after lyophilization at either 4° C. or at room temperature was evaluated by thermal denaturation utilizing the Tycho (NanoTemper) and compared to reference antigen. This approach involves measuring the intrinsic fluorescence (detected at both 350 nm and 330 nm) given off by tryptophan and tyrosine residues as a thermal ramp is applied to the sample and the proteins begin to unfold. Changes in fluorescence signal (Ratio 350 nm/330 nm) indicate transitions in the folding status of a protein and the temperature at which a transition occurs is called the inflection temperature (Ti). Comparison of the thermal denaturation profile and Ti of samples stored under different conditions provides a rapid biophysical assessment of the structural integrity of the protein.

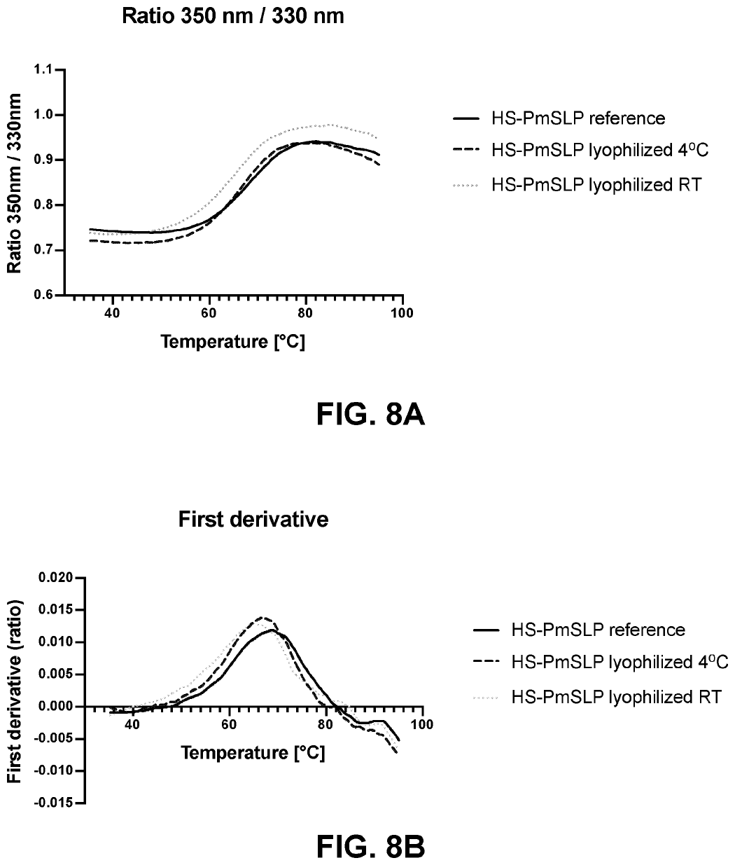

PmSLP-3 protein that was lyophilized and stored at 4° C. or room temperature (RT) for one year was compared to reference PmSLP-3 protein, a freshly prepared sample at a concentration of 100 μg/mL in buffer (phosphate buffered saline, pH 7.4). Lyophilized proteins were reconstituted at 100 μg/mL in the same buffer prior to analysis. The thermal denaturation profiles and Tis showed no appreciable change after storage at either room temperature or 4° C. for one year when evaluating the fluorescence signals (FIG. 8A; FIG. 8B), indicating that the lyophilized proteins maintained structural integrity for this duration.

The effect of antigen lyophilization and storage at 4° C. was further evaluated in vivo in a mouse infection model. PmSLP-3 protein (SEQ. ID NO: 20) was formulated as a vaccine using 20 μg of protein mixed with Montanide™ Gel 02 (Seppic) combined with Poly (I:C) (Invivogen) to a final volume of 100 μl per dose. 4-6-week-old male C57BI/6 mice (Charles River) were given two doses of vaccine at three-week intervals via sub-cutaneous injection. Vaccine group 1 received formulation that was prepared fresh prior to each dose using PmSLP-3 protein aliquots that had been stored at −80° C. Vaccine group 2 received formulation that was prepared fresh prior to each dose using PmSLP-3 protein aliquots that had been lyophilized and stored at 4° C. Vaccine group 3 received formulation that was prepared fresh prior to the first dose using PmSLP-3 protein that had been stored at −80° C.; the mixed formulation was then stored at 4° C. for three weeks and administered again during the second dose.

Six weeks after the second dose, anaesthetized animals were challenged via intra-peritoneal injection with approximately 10⁴ CFU of log-phase P. multocida strain H229 (antigen-matched porcine isolate, the confirmed sequence of the PmSLP in this strain is defined in SEQ. ID NO: 24). Mice were monitored every 6-12 hours post infection for clinical symptoms including lethargy, breathing, movement, dehydration, diarrhea, posture, and weight loss. A combined clinical score of 10 or higher was considered as clinical endpoint, at which point mice were humanely euthanized (FIG. 9). All mice that received any of the PmSLP-3 vaccines were fully protected from infection, compared to all animals that received only adjuvant which reached endpoint at 21 hours post infection (FIG. 9A). Clinical scores for individual mice that received adjuvant only (FIG. 9B), Vaccine 1 (freshly prepared vaccine with protein that was stored at −80° C. until immediately prior to each dose; FIG. 9C), Vaccine 2 (freshly prepared vaccine with protein that was lyophilized and stored at 4° C. until immediately prior to each dose; FIG. 9D), or Vaccine 3 (vaccine formulated prior to dose 1 with protein stored at −80° C. and then remaining vaccine stored at 4° C. for three weeks until the second dose; FIG. 9E).

### Example 7—Immunogenicity of PmSLP-1 in Cattle

This Example illustrates the immunogenicity of PmSLP vaccine in a food production animal that is impacted by P. multocida infection. In this Example, two different breeds of cattle were used to evaluate the immunogenicity of a PmSLP-1 vaccine. A PmSLP-1 vaccine was chosen for this Example in the cattle host since this variant is expressed by the vast majority of bovine respiratory disease (BRD) causing P. multocida isolates.

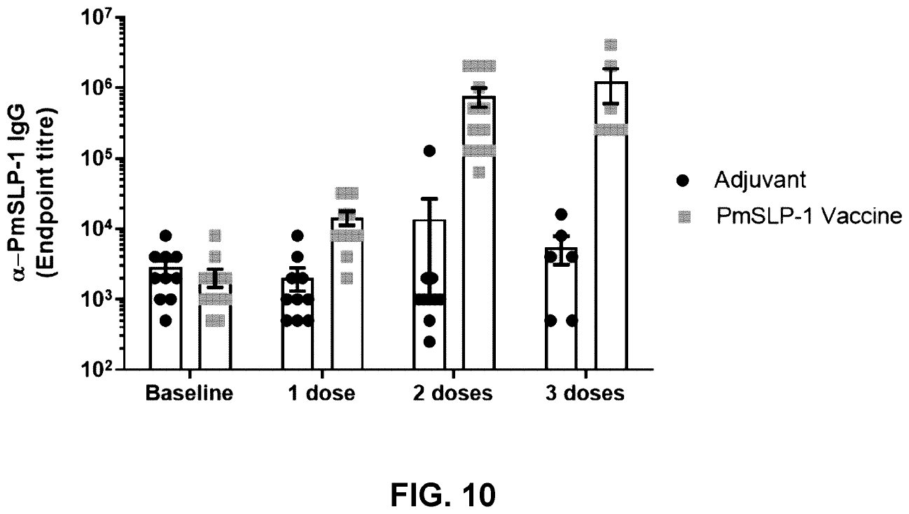

Healthy zebu breed cattle (age 4-6 months old) that were seronegative against all serotypes of P. multocida were randomized into two groups. 200 μg of lyophilized PmSLP-1 protein (SEQ. ID NO: 14) was reconstituted with PBS directly prior to vaccination and formulated with 30 μg of Poly (I:C) (Invivogen) and 20% v/v Montanide™ Gel 02 (Seppic) in a final volume of 2 mL per dose. Animals were randomized into groups and received three doses, three weeks apart, of either PmSLP-1 vaccine or adjuvant alone via sub-cutaneous injection. Blood was collected prior to vaccination (baseline), and approximately 2-3 weeks after the first, second and third dose. Serum samples were evaluated for the presence a-PmSLP-1 IgG using ELISA (enzyme linked immunosorbent assay). FIG. 10 shows the endpoint titre of cattle immunized with either PmSLP-1 vaccine or adjuvant. As shown, there is a detectable increase in PmSLP-1 specific antibody after one dose, which is substantially increased after two doses. There is minimal boosting effect with a third dose.

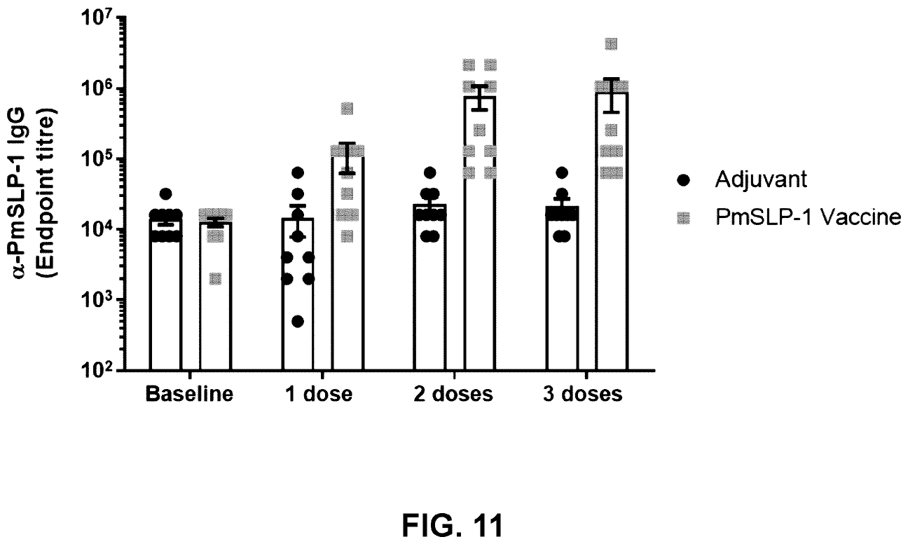

A second cattle immunization was performed with healthy beef cattle (age approximately 10 months old) that were seronegative against all serotypes of P. multocida. Animals were randomized into two groups of 9 animals each. Animals received either three doses of PmSLP-1 (SEQ. ID NO: 14) vaccine or adjuvant. Vaccine was prepared prior to each immunization by mixing 200 μg of PmSLP-1 protein that was stored at −80° C. with 30 μg of Poly (I:C) (Invivogen) and 20% v/v Montanide™ Gel 02 (Seppic) in a final volume of 2 mL per dose. Animals were vaccinated in three-week intervals intra-muscularly and bleeds were taken prior to the first immunization (baseline) and 2-3 weeks after the first, second and third dose. Serum was evaluated for the presence of a-PmSLP-1 IgG. FIG. 11 shows the endpoint titre of cattle immunized with either PmSLP-1 vaccine or adjuvant at baseline and after one, two, or three doses of vaccine. As shown, there is a detectable increase in specific antibody after one dose, which is substantially increased after two doses, with again very minimal boosting effect of a third dose.

Overall, the PmSLP-1 vaccine was immunogenic in both cattle breeds, with titres peaking after 2 doses of vaccine administered either intramuscularly or subcutaneously with this formulation.

### Example 8—Protection of PmSLP-3 in Cattle Against a Serogroup B Strain of P. multocida

This Example illustrates the immunogenicity, safety, and protective efficacy of PmSLP vaccine in a food production animal that is impacted by P. multocida infection. In this Example, zebu breed cattle were used to evaluate the immunogenicity and safety of two PmSLP-3 vaccine formulations, as well as to evaluate the protective efficacy of this vaccine against lethal challenge of Serogroup B P. multocida. A PmSLP-3 vaccine was chosen for this Example in the cattle host since this variant is expressed by all of the known hemorrhagic septicemia (HS) causing P. multocida isolates.

Healthy zebu breed cattle (aged 4-6 months old) that were seronegative against all serotypes of P. multocida were randomized into three groups. 200 μg of PmSLP-3 (SEQ. ID NO 20) was reconstituted in PBS directly prior to vaccination and formulated with either 1 mg of aluminum hydroxide (Alhydrogel, Sigma-Aldrich) or with 30 μg of Poly (I:C) (Invivogen) and 20% v/v Montanide™ Gel 02 (Seppic) in a final volume of 2 mL per dose. A control group that received only adjuvant was used as the negative control. Animals were vaccinated twice in three-week intervals sub-cutaneously and bleeds were taken prior to the first immunization (baseline), prior to the second immunization (post first dose), and prior to challenge (post second dose). Serum was evaluated for the presence of a-PmSLP-1 IgG. Injection sites were monitored for local reactions after vaccination. 14 days after the booster dose, cattle were challenged sub-cutaneously with 4.4×10⁴ CFU/mL of a serogroup B strain of P. multocida and monitored for 8 days after infection.

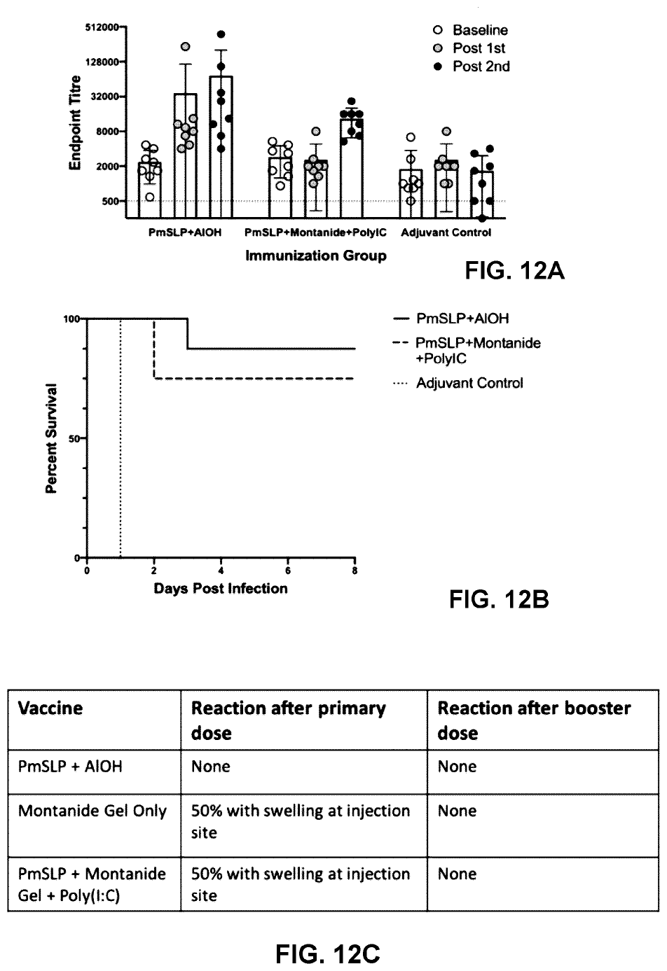

FIG. 12A shows the endpoint titre of cattle immunized with PmSLP-3 formulated with aluminum hydroxide, PmSLP-3 formulated with Montanide Gel 02+Poly (I:C), or with adjuvant alone. As shown, there is detectable serum IgG against PmSLP-3 after two doses of vaccine with both PmSLP-3 vaccine formulations, while PmSLP-3 formulated with aluminum hydroxide elicited detectable serum IgG against PmSLP-3 after the first dose. FIG. 12B shows survival of cattle after challenge of serogroup B P. multocida. As shown, zebu cattle that received adjuvant only were fully vulnerable to infection and succumbed one day post infection. In comparison, 87.5% of cattle (7/8) vaccinated with PmSLP-3 formulated with aluminum hydroxide survived challenge, and 75% of cattle (6/8) vaccinated with PmSLP-3 formulated with Montanide Gel 02+Poly (I:C) survived challenge. FIG. 12C shows a table containing reactogenicity after one or two doses of vaccines. As shown, PmSLP-3 formulated with aluminum hydroxide showed no local reactions following either dose, while PmSLP-3 formulated with Montanide Gel 02+Poly (I;C) or Montanide Gel 02 alone caused local swelling in 50% of animals after the first dose.

Overall, PmSLP-3 vaccines formulated with either aluminum hydroxide or Montanide Gel 02+Poly (I:C) were safe, immunogenic, and protective in zebu cattle after two doses of vaccine when delivered sub-cutaneously.

### Example 9—Evaluation of the Pathogenic Capacity of the Brazilian Field Strain of Pasteurella multocida Serogroup a in Mice

### Mouse Challenging Study

The objective of the study described in this Example 9 was to determine the pathogenicity of two Pasteurella multocida isolates, namely AFK 143/23 Cluster 2 and AFK 283/23 Cluster 4.2 in mice.

### Bacterial Growth and Infectious Dose Formulation

Three AFK isolates of P. multocida serogroup A (AFK AFK 300/23 PmSLP-1, AFK 143/23 PmSLP-2 and AFK 283/23 PmSLP-4) were used in this study. Bacterial isolates were cultivated overnight at 37° C. on defibrinated blood agar plates. From the plate the bacteria were inoculated into an Erlenmeyer containing liquid PPLO (pleuropneumonia-like organisms, Oxoid, USA) media; the initial optical density (OD) was adjusted at 0.1 assessed by spectrophotometry (filter of 660 nm). Thereafter, the bacteria were incubated at 37° C. under constant agitation (180 rpm) until the OD reached 0.5. The bacteria were then collected by centrifugation (4,000×g for 15 min at 4° C.) and the pellet was resuspended in phosphate buffered saline (PBS) pH 7.4. The absolute number of bacteria in the suspension was determined by flow cytometry (Baldasso, D. Z., Guizzo, J. A., Dazzi, C. C., Paraboni Frandoloso, G. C., Feronato, C., von Berg, S., Carvalho Guedes, R. M., Wilson, H. L., Kreutz, L. C., Frandoloso, R., 2023. Development and validation of a flow cytometry antibody test for Lawsonia intracellularis. Front Immunol 14, 1145072) and the bacteria; viability was simultaneously assessed by staining with propidium iodine (PI). All of the assays were performed in triplicates. Three infectious doses were prepared containing 10², 10³, and 10⁴ P. multocida. The bacteria were then diluted in PBS (pH 7.4) to a final volume of 0.2 mL.

### Mice Experimental Infection

Forty-five C57BL/6 mice 8-weeks old were used in this study. Mice were randomly assigned equally in 9 groups and housed in the Central Animal Facility of the University of Passo Fundo under a photoperiodism of 12/12 h light/dark, commercial ratio and water at libitum. Nine groups of mice were intraperitoneally infected with P. multocida serogroup A as illustrated in FIG. 13A. The inoculum was suspended in 0.2 mL given by the intraperitoneal route. The number of dead/alive mice was monitored afterwards.

### Results

As illustrated in FIG. 13B, all challenged mice died after the inoculation of assessed strains. Animals challenged with the 10³ and 10⁴ bacteria died significantly (p<0.05) earlier than the animals challenged with the 10² bacteria. The 100% lethal dose (LD₁₀₀) was defined as 10² bacteria for the three strains analyzed.

### Example 10—Clinical Efficacy Study in Mice. Animals Immunized with PmSLP2-Based Vaccine and Challenged with P. multocida Strain 143/23

### Animal Model and Groups

Ten C57BL/6 mice were equally divided into two groups: one group of 5 mice were immunized with a vaccine formulated with PmSLP2 recombinant protein (SEQ. ID NO: 95), and the other 5 mice were inoculated with PBS plus adjuvant.

### Vaccine Production and Immunization Protocol

The immunizing antigen (PmSLP2) was diluted in PBS (pH 7.4) and mixed with Emulsigen D (MVP Adjuvants, Teaneck, USA) to a final concentration of 20% adjuvant and 0.1 mg of antigen/mL. The antigen plus adjuvant mixture was homogenized on a magnetic agitator (600 rpm) for 2 h and then stored refrigerated (4° C.) until use. The vaccines were packaged in flasks containing 25 doses of 2 mL (final volume). The volume of the dose defined in this step was used in the experiment carried out on the pigs.

Mice immunization was performed according to standard protocols. Briefly, 5 mice were inoculated intraperitoneally with 0.2 mL of the vaccine (20 μg of PmSLP2) which corresponded to 10% of the immunization dose used for pigs; the other 5 mice received sterile PBS plus adjuvant (control group). The immunization protocol was repeated in all mice with 14 days interval (booster injection).

### Blood Sampling

Blood samples were collected from all mice prior to the first immunization (day 0), at the time of revaccination (day 14) and then again at day 28. In addition, at day 28 all mice were inoculated with the challenging Pasteurella multocida serogroup A as described below.

### IgG Titration of Mice Blood

The titres of anti-P. multocida antibodies was evaluated by indirect ELISA. Briefly, plates were sensitized with PmSLP-2 (1 μg/well) overnight (4° C.). Then the wells were washed and blocked (37° C.) for 1 h with 5% skim milk (SK) in PBS containing 0.05% Tween-20 (PBS-T). After washing again, mouse sera were serially diluted (in PBS-T 1% SK) and added to the plates and incubated for 1 h at 37° C. The sera were removed, and the plates were washed once more prior to the addition of peroxidase-conjugated anti-mouse IgG. After incubating for 1 h (37° C.) the plates were washed again prior to the addition of substrate (3,3′,5,5′-Tetramethylbenzidine Liquid Substrate). The reaction was stopped, and the plates were read on an ELISA microplate reader (450 nm filter). Serum samples were considered positive when the OD reading of the sample was at least 2 times higher than the average OD of the negative control samples.

### Bacteria Inoculum and Experimental Challenge

The bacterial isolate (AFK 143/23-Pasteurella multocida serogroup A, PmSLP cluster 2) was cultivated overnight at 37° C. on defibrinated blood agar plates. From the plate the bacteria were inoculated into an Erlenmeyer containing liquid PPLO (pleuropneumonia-like organisms, Oxoid, USA) media; the initial optical density (OD) was adjusted at 0.1 assessed by spectrophotometry (filter of 660 nm). Then, the bacteria were incubated at 37° C. under constant agitation (180 rpm) until the OD reached 0.5. The bacteria were then collected by centrifugation (4,000 g for 15 min at 4° C.) and the pellet resuspended in phosphate buffered saline (PBS) pH 7.4. The absolute number of bacteria in the suspension was determined by flow cytometry (Baldasso et al., 2023) and the bacterial viability was simultaneously assessed by staining with propidium iodine (PI). All of the assays were performed in triplicates. For the mice challenging study, the bacteria were then diluted in PBS (pH 7.4) to contain 102 bacteria per infectious dose.

All mice were challenged at day 28 (14 days after boosting immunization). The challenging inoculum (Lethal Dose—LD_{100%}) was administered intraperitoneally and contained 102 bacteria (AFK 143/23 Cluster 2 of PmSLP from P. multocida) on a final volume of 200 μL. After the challenge the mice were monitored twice daily for up to seven days.

### Results

### Serology

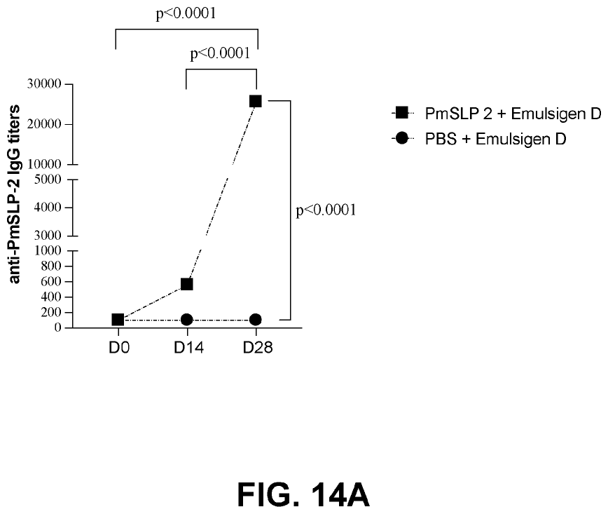

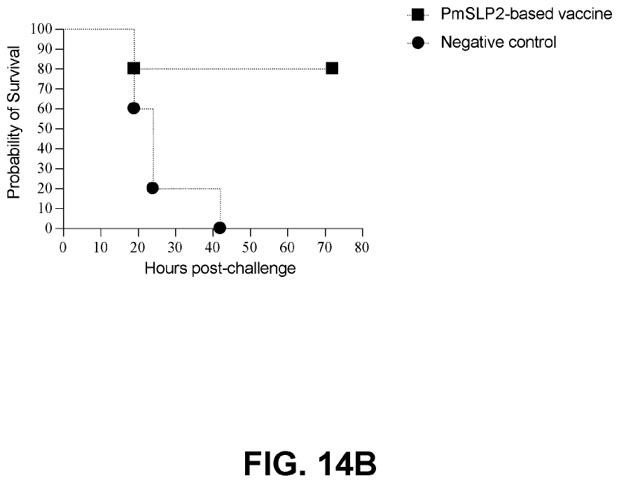

As illustrated in FIG. 14A, the PmSLP2-based vaccine induced anti PmSLP-2 IgG production in mice. Between the first and second doses of the vaccine, a slight increase of anti-PmSLP-2 IgG titers was observed, however, 14 days after the second dose (D28), the IgG titres increased significantly (p>0.0001) in relation to the two previous moments analyzed (DO and D14). At D28 the IgG titers were 45 times higher than those observed at D14. At that same moment, the levels of IgGs were different (p>0.0001) in comparison to non-vaccinated animals. Therefore, the results of this analysis demonstrate that the PmSLP-2 recombinant protein in combination with the oil-based adjuvant Imulsigen D is highly immunogenic in mice.

### Clinical Efficacy

As illustrated in FIG. 14B, the PmSLP2-based vaccine induced clinical protection in 80% of mice challenged with a lethal dose of P. multocida A (strain AFK 143.23); this level of protection was statistically superior in relation to the negative control group (0% survival). These results demonstrate that the PmSLP antigen is protective against lethal challenge with Pasteurella multocida serogroup A.

### Example 11—Evaluation of the Pathogenic Capacity of the Brazilian Field Strain of Pasteurella multocida Serogroup a in Pigs

The objective of the study described in this Example 11 was to determine the pathogenicity of two Pasteurella multocida isolates, AFK 143/23 (PmSLP-2) and AFK 283/23 (PmSLP-4) in pigs.

### Pig Experimental Infection

Twelve colostrum-deprived pigs were used in this study. Colostrum-deprived piglets were produced as described previously (Guizzo, J. A., Chaudhuri, S., Prigol, S. R., Yu, R. H., Dazzi, C. C., Balbinott, N., Frandoloso, G. P., Kreutz, L. C., Frandoloso, R., Schryvers, A. B., 2018. The amino acid selected for generating mutant TbpB antigens defective in binding transferrin can compromise the in vivo protective capacity. Scientific Reports 8, 7372). For the challenging study all pigs (63 days-old) were anesthetized with a cocktail of 0.3 mg/kg of acepromazine (Syntec do Brasil, Brazil), 0.3 mg of midazolam (Laboratório Teuto Brasileiro, Brazil) and 15 mg/kg of Ketamine (Ceva Santé Animale, Brazil), administered by the intramuscular route. Then, the anesthetized pigs were held in an upright position and intranasally inoculated. Each P. multocida isolate was used to inoculated 6 pigs: 3 pigs with 108 and 3 pigs with 109 bacteria in a final volume 3 ml of PBS, pH 7.4. After challenging pigs were monitored for the presence of clinical signs up to 10 days post challenging.

### Results

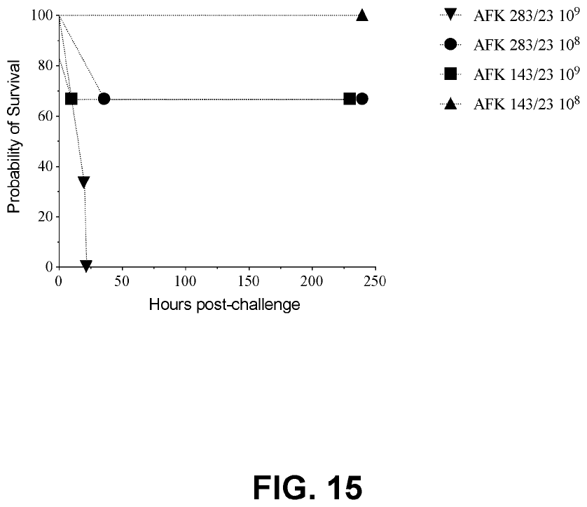

Two out of the three pigs inoculated with P. multocida AFK 283/23 (109 bacteria/pig) were dead by 20 h post challenging; the other pig of this group died 2 hours later with severe respiratory distress. Pigs challenged with the lower dose (108 bacteria/pig) of the same strain had severe dyspnea within 12 hours post challenging and one piglet was dead by 36 h post challenging. The other two pigs had severe respiratory distress up to 5 days post challenging only and ameliorated afterwards (see: FIG. 15).

Within the cohort of pigs challenged with AFK143/23 strain, only one animal inoculated with 109 bacteria died by 20 hours post challenge; the remaining pigs in this group had respiratory distress (dyspnea, coughing, prostration) up to 4 days post challenging. Clinical signs regressed afterwards (See: FIG. 15).

During necropsy, all animals that died after the experimental infection or that were euthanized at the end of the study showed severe bronchopneumonia with extensive necro-hemorrhagic areas in the diaphragmatic lobes. Intense pleurisy was observed in all animals that were euthanized at the end of the study. Pasteurella multocida A was recovered in pure and abundant culture from the lungs of all challenged animals.

Considering the infection profile of P. multocida A in the field, we concluded that an appropriate infection dose for the AFK 283/23 strain is 108 bacteria, and for the 143/23 strain, 109 bacteria. Using these doses, it was possible to induce respiratory clinical signs and typical lung macroscopic lesions in the pigs studied.

Thus, this Example 11, demonstrates that AFK 143/23 (PmSLP-2) and AFK 283/23 (PmSLP-4) strains are pathogenic for pig and act as primary agents of severe pulmonary disease in this animal species. Studies published by other Brazilian researchers have described the circulation in Brazil of strains of P. multocida serogroup A that are highly pathogenic and capable of triggering severe pneumonia in swine (Chitarra, C. S., Oliveira Filho, J. X., Mores, N., Silva, M., Candido, S. L., Cezarino, P. G., Nakazato, L., Dutra, V., 2018. Identification of Pasteurella multocida transcribed genes in porcine lungs through RNAseq. Microbial pathogenesis 122, 180-183; Oliveira Filho, J. X., Mores, M. A. Z., Rebellato, R., Kich, J. D., Cantao, M. E., Klein, C. S., Guedes, R. M. C., Coldebella, A., Barcellos, D., Mores, N., 2018. Pathogenic variability among Pasteurella multocida type A isolates from Brazilian pig farms. BMC veterinary research 14, 244).

Additionally, it was evaluated whether sera from convalescent animals had antibodies against PmSLP-2 and PmSLP4 proteins. This analysis was carried out to understand whether these antigens are naturally expressed by P. multocida A during the infection process, and consequently, whether these proteins stimulate the production of antibodies in pigs.

Interestingly, all pigs that survived the challenge with strain AFK 143/23 (n=5) and AFK 283/23 (n=2) had IgG titers against PmSLP proteins. The presence of these antibodies was not observed before the challenge; therefore, the antibodies are specific. This result demonstrates: i) that the PmSLP protein is an antigen that participates in the pathogenesis of P. multocida infection in pig; ii) that it is an antigen naturally accessible to B lymphocytes, and therefore, immunogenic, and antigenic. These results the utility of PmSLP based antigens in the preparation of a vaccine against P. multocida.

### Example 12—Efficacy Study in Mice of Vaccines Based on the PmSLP Proteins

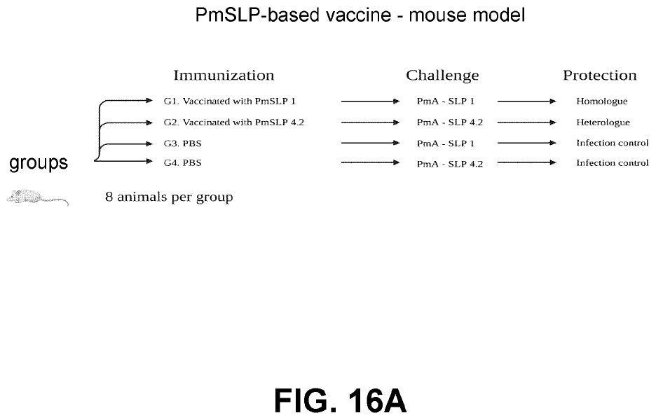

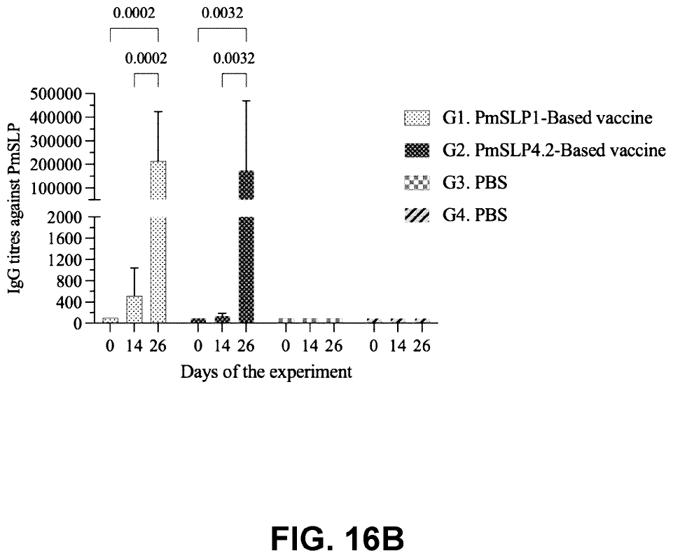

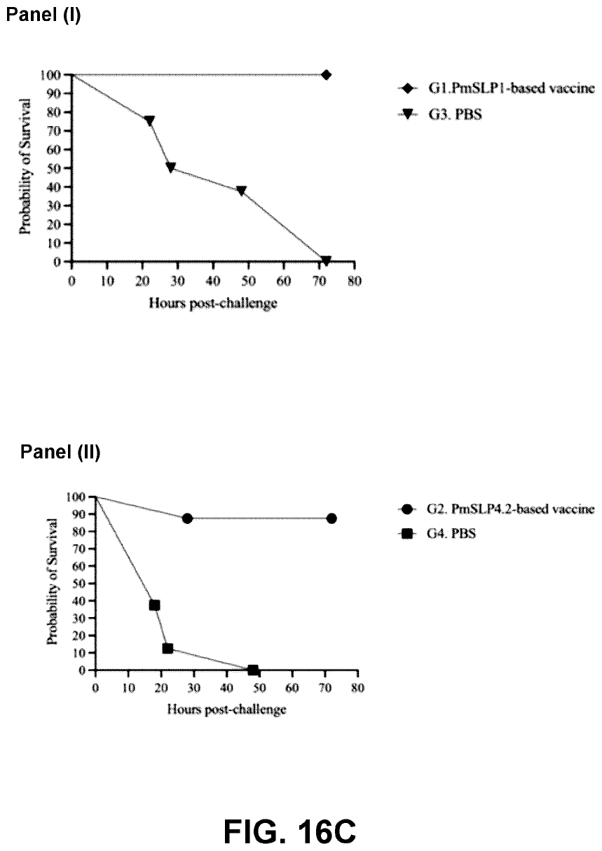

This Example 12 describes the evaluation of the protective capacity of two other vaccines, one based on PmSLP-1 and another in PmSLP-4.2. In this case, as illustrated in FIG. 16A, mice were challenged with P. multocida strains that expressed PmSLPs homologous to those used in the vaccine formulation.

For this purpose, 32 male and female C57BL/6 mice were used, equally distributed in 4 groups (G1 to G4). The mice were housed in the Central Animal Facility of the University of Passo Fundo under a 12/12 light/dark photoperiod regimen. The antigen and immunization protocol that was used are depicted in FIG. 16A. Vaccines were formulated as described in Example 10. Challenges with the homologous PmSLP expressing bacteria were performed at day 28 (14 days after the booster vaccination) and were, furthermore, performed intraperitoneally using 102 bacteria/mouse in a final volume of 0.2 mL.

### Results

As illustrated in FIG. 16B, the two vaccines applied to mice stimulated the production of systemic antibodies (serology conducted as described in Example 10). In this instance, sera samples (pre-challenge moment) were collected 12 days after revaccination, and upon collection, the IgG titers were significantly higher than those observed at the pre-vaccination moment in all animals. Therefore, all vaccines were immunogenic and deemed safe, as none of the animals displayed systemic reactions during the post-vaccination period.

As illustrated in FIG. 16C, P. multocida A strains AFK 300/23 (PmSLP-1) and AFK 283/23 (PmSLP-4.2) were lethal to mice that had been inoculated with PBS (Groups 3 and 4). By contrast, the vaccine based on the PmSLP-1 protein (SEQ. ID NO: 93) conferred 100% protection against the AFK 300/23 strain (Group 1). The vaccine based on the PmSLP-4.2 protein (SEQ. ID NO: 97) protected 87.5% of mice challenged with the AFK 283/23 strain (Group 2). These data, together with those presented in FIG. 14B, demonstrate that PmSLP1, 2 and 4.2 proteins induce >80% protection against challenges using the 100% lethal dose of pathogenic P. multocida A. The protection shown in this study can be said to be homologous, with the vaccine antigen belonging to the same PmSLP cluster of the challenge strain.

1. A veterinary vaccine formulation for the prevention, treatment or amelioration of P. multocida infection in a food production animal susceptible to P. multocida infection, the vaccine formulation comprising an effective amount of at least one PmSLP protein, or an immunogenically equivalent portion thereof, together with a veterinary pharmaceutically acceptable excipient, carrier, or diluent, and, optionally, a veterinary pharmaceutically acceptable adjuvant.

2. (canceled)

3. A veterinary vaccine formulation according to claim 1, wherein the vaccine formulation comprises a P. multocida PmSLP protein, or an immunogenically equivalent portion thereof, selected from the group of phylogenetic clusters consisting of PmSLP-1, PmSLP-2, PmSLP-3, PmSLP-4.1, and PmSLP-4.2, wherein the selected P. multocida PmSLP protein, or an immunogenically equivalent portion thereof, belongs to the same phylogenetic cluster as a PmSLP protein present in the infecting P. multocida strain.

4.-6. (canceled)

7. A veterinary vaccine formulation according to claim 1, wherein the food production animal is a porcine animal susceptible to infection by a P. multocida strain causing porcine atrophic rhinitis (PAR), and the vaccine formulation comprises a P. multocida PmSLP protein from a PAR causing P. multocida strain, or a pneumonic pasteurellosis causing P. multocida strain, respectively, or an immunogenically equivalent portion thereof, wherein the PmSLP protein is selected from the group of phylogenetic clusters consisting of PmSLP-2, PmSLP-4.1, and PmSLP-4.2, wherein the selected P. multocida PmSLP protein, or an immunogenically equivalent portion thereof, belongs to the same phylogenetic cluster as a PmSLP protein present in the infecting P. multocida strain.

8.-9. (canceled)

10. A veterinary vaccine formulation according to claim 1, wherein the vaccine formulation further is of a P. multocida strain belonging to a serogroup selected from the group consisting of serogroup A, B, D, E, and F, wherein the serogroup is the same as the serogroup of the infecting P. multocida strain.

11. A veterinary vaccine formulation according to claim 1, wherein the at least one PmSLP protein, or immunologically equivalent portion thereof, is a protein expressed by a nucleic acid sequence selected from the group of nucleic acid sequences consisting of:
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence having at least 70% identity with any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a chimeric nucleic acid obtained by a fusion between at least two nucleic acid sequences of (a), (b), (c), and (d), or a portion thereof;
(f) a nucleic acid sequence that is complementary to any one of the nucleic acid sequences of (a);
(g) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(h) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereto; and
(i) a nucleic acid sequence that hybridizes under stringent conditions to any one of the nucleic acid sequences set forth in (a), (b), (c), (d), (e), (f), (g), or (h).

12.-19. (canceled)

20. A veterinary vaccine formulation according to claim 1, wherein the food production animal is a porcine species, and the P. multocida infection causes pneumonic pasteurellosis or PAR, and wherein the PmSLP protein comprises SEQ. ID NO: 4, SEQ. ID NO: 8, SEQ. ID NO: 10, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 75, SEQ. ID NO: 77, or SEQ. ID NO: 91, or an immunogenically equivalent portion thereof, to prevent or ameliorate a P. multocida infection causing pneumonic pasteurellosis or PAR.

21.-23. (canceled)

24. A veterinary vaccine formulation according to claim 1, wherein the veterinary vaccine formulation is a cross-protective vaccine formulation comprising a PmSLP protein, or immunogenically equivalent portion thereof, obtained from a first P. multocida strain, and the vaccine formulation is a formulation for the administration to the food production animal to prevent or ameliorate an infection caused by another P. multocida strain.

25. A veterinary vaccine formulation according to claim 1, wherein the vaccine formulation is substantially free of other P. multocida constituents.

26.-28. (canceled)

29. A veterinary vaccine formulation according to claim 1, wherein the vaccine formulation comprises from about 0.001% to about 20% by weight of the PmSLP protein or the immunogenically equivalent portion thereof, and a veterinary pharmaceutically acceptable adjuvant constituting from about 0.1% to about 60% by weight or volume of the vaccine formulation.

30. A veterinary vaccine formulation according to claim 1, wherein the vaccine formulation comprises a second P. multocida PmSLP protein, or immunologically equivalent portion thereof.

31. (canceled)

32. A veterinary vaccine formulation according to claim 30, wherein the second P. multocida PmSLP protein, or immunologically equivalent portion thereof, belongs to a different phylogenetic cluster as the first P. multocida PmSLP protein, or immunologically equivalent portion thereof or, optionally, the same phylogenetic cluster as the first P. multocida PmSLP protein, or immunologically equivalent portion thereof.

33. A veterinary vaccine formulation according to of claim 32, wherein the fusion polypeptide comprises a fusion polypeptide selected from the group consisting of a (i) PmSLP protein, or immunologically equivalent portion thereof, belonging to phylogenetic cluster 1 and a PmSLP protein, or immunologically equivalent portion thereof, belonging to phylogenetic cluster 3; (ii) PmSLP protein, or immunologically equivalent portion thereof, belonging to phylogenetic cluster 1 and a PmSLP protein, or immunologically equivalent portion thereof, belonging to phylogenetic cluster 2; (iii) a PmSLP protein, or immunologically equivalent portion thereof, belonging to phylogenetic cluster 1 and a PmSLP protein, or immunologically equivalent portion thereof, belonging to phylogenetic cluster 4.1; and (iv) a PmSLP protein, or immunologically equivalent portion thereof, belonging to phylogenetic cluster 1 and a PmSLP protein, or immunologically equivalent portion thereof, belonging to phylogenetic cluster 4.2.

34. A veterinary vaccine formulation according to claim 33, wherein the second P. multocida PmSLP protein, or immunologically equivalent portion thereof, is obtained from a P. multocida strain belonging to the same or a different serogroup as the P. multocida strain of the first P. multocida PmSLP protein, or immunologically equivalent portion thereof.

35.-37. (canceled)

38. A method for prevention, treatment or amelioration of Pasteurella multocida (P. multocida) infection in of a food production animal susceptible to P. multocida infection, the method comprising administering to the food production animal a veterinary vaccine formulation comprising a P. multocida PmSLP protein, or an immunogenically equivalent portion thereof, wherein the vaccine formulation is administered in an effective amount to prevent, treat or ameliorate the P. multocida infection.

39.-45. (canceled)

46. The method according to claim 38, wherein the food production animal susceptible to infection by a P. multocida strain is a porcine animal, and wherein the veterinary vaccine formulation administered in an effective amount to the porcine animal comprises at least one PmSLP protein from a P. multocida causing porcine atrophic rhinitis (PAR) or pneumonic pasteurellosis, or an immunogenically equivalent portion thereof.

47. (canceled)

48. A method according to claim 38, the food production animal is a porcine animal susceptible to infection by a P. multocida strain causing porcine atrophic rhinitis (PAR) or pneumonic pasteurellosis, and the vaccine formulation comprises a P. multocida PmSLP protein from a PAR causing P. multocida strain or a pneumonic pasteurellosis causing P. multocida strain, respectively, or an immunogenically equivalent portion thereof, wherein the PmSLP protein is selected from the group of phylogenetic clusters consisting of PmSLP-1, PmSLP-2, PmSLP-3, PmSLP-4.1, and PmSLP-4.2, wherein the selected P. multocida PmSLP protein, or an immunogenically equivalent portion thereof, belongs to the same phylogenetic cluster as a PmSLP protein present in the infecting P. multocida strain.

49.-50. (canceled)

51. A method according to claim 38, wherein the vaccine formulation comprises at least one P. multocida PmSLP protein, or an immunogenically equivalent portion thereof, selected from a protein encoded by a nucleic acid sequence selected from the group of nucleic acid sequences consisting of:
(a) SEQ. ID NO: 1, SEQ. ID NO: 3, SEQ. ID NO: 5, SEQ. ID. NO: 7, SEQ. ID NO: 9, SEQ. ID NO: 11, SEQ. ID NO: 13, SEQ. ID NO: 15, SEQ. ID NO: 17, SEQ. ID. NO: 19, SEQ. ID. NO: 21, SEQ. ID. NO: 23, SEQ. ID. NO: 25, SEQ. ID. NO: 27, SEQ. ID NO: 29, SEQ. ID NO: 31, SEQ. ID NO: 33, SEQ. ID NO: 35, SEQ. ID NO: 37, SEQ. ID NO: 39, SEQ. ID NO: 50, SEQ. ID NO: 52, SEQ. ID NO: 54, SEQ. ID NO: 56, SEQ. ID NO: 58, SEQ. ID NO: 60, SEQ. ID NO: 62, SEQ. ID NO: 64, SEQ. ID NO: 66, SEQ. ID NO: 68, SEQ. ID NO: 70, SEQ. ID NO: 72, SEQ. ID NO: 74, SEQ. ID NO: 76, SEQ. ID NO: 78, SEQ. ID NO: 80, SEQ. ID NO: 82, SEQ. ID NO: 84, SEQ. ID NO: 86, SEQ. ID NO: 88, SEQ. ID NO: 90, SEQ. ID NO: 92, SEQ. ID NO: 94, or SEQ. ID NO: 96;
(b) a nucleic acid sequence having at least 70% identity with any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a chimeric nucleic acid obtained by a fusion between at least two nucleic acid sequences of (a), (b), (c), and (d), or a portion thereof;
(f) a nucleic acid sequence that is complementary to any one of the nucleic acid sequences of (a);
(g) a nucleic acid sequence encoding a polypeptide having any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereof;
(h) a nucleic acid sequence that encodes a functional variant of any one of the amino acid sequences set forth in SEQ. ID NO: 2, SEQ. ID NO: 4, SEQ. ID NO: 6, SEQ. ID. NO: 8, SEQ. ID NO: 10, SEQ. ID NO: 12, SEQ. ID NO: 14 SEQ. ID NO: 16, SEQ. ID NO: 18, SEQ. ID. NO: 20, SEQ. ID. NO: 22, SEQ. ID. NO: 24, SEQ. ID. NO: 26, SEQ. ID. NO: 28, SEQ. ID NO: 30, SEQ. ID NO: 32, SEQ. ID NO: 34, SEQ. ID NO: 36, SEQ. ID NO: 38, SEQ. ID NO: 40, SEQ. ID NO: 51, SEQ. ID NO: 53, SEQ. ID NO: 55, SEQ. ID NO: 57, SEQ. ID NO: 59, SEQ. ID NO: 61, SEQ. ID NO: 63, SEQ. ID NO: 65, SEQ. ID NO: 67, SEQ. ID NO: 69, SEQ. ID NO: 71, SEQ. ID NO: 73, SEQ. ID NO: 75, SEQ. ID NO: 77, SEQ. ID NO: 79, SEQ. ID NO: 81, SEQ. ID NO: 83, SEQ. ID NO: 85, SEQ. ID NO: 87, SEQ. ID NO: 89, SEQ. ID NO: 91, SEQ. ID NO: 93, SEQ. ID NO: 95, or SEQ. ID NO: 97, or an immunogenically equivalent portion thereto; and
(i) a nucleic acid sequence that hybridizes under stringent conditions to any one of the nucleic acid sequences set forth in (a), (b), (c), (d), (e), (f), (g), or (h).

52.-56. (canceled)

57. A method according to claim 38, wherein the vaccine formulation comprises a veterinary pharmaceutically acceptable excipient, carrier, or diluent and, optionally, a veterinary pharmaceutically acceptable adjuvant.

58. A method for preparing a veterinary vaccine formulation for the prevention, treatment or amelioration of P. multocida infection in a food production animal susceptible to P. multocida infection, the method comprising:
(i) diagnosing a P. multocida infection in a food production animal;
(ii) identifying the phylogenetic cluster to a which a PmSLP protein present in the infecting P. multocida belongs, the phylogenetic cluster being selected from PmSLP-1, PmSLP-2, PmSLP-3, PmSLP-4.1 or PmSLP-4.2; and
(iii) preparing a vaccine formulation comprising a P. multocida PmSLP protein, or immunogenically equivalent portion thereof, which belongs to the identified phylogenetic cluster together with a veterinary pharmaceutically acceptable adjuvant to form a veterinary vaccine formulation comprising an effective amount of the P. multocida PmSLP protein or the immunogenically equivalent portion thereof to treat a food production animal susceptible to P. multocida infection.

59. A method according to claim 58, wherein the method additionally comprises identifying the serogroup of the infecting P. multocida strain, the serogroup being selected from the group consisting of serogroup A, B, D, E, and F, and the vaccine being prepared using a P. multocida PmSLP protein, or an immunogenically equivalent portion thereof, from the same or another P. multocida strain belonging to the selected serogroup.

60.-66. (canceled)


SEQUENCE LISTING (97 sequences)

SEQ ID NO: 1 - DNA, 954 bp, Pasteurella multocida
  source 1..954: mol_type = genomic DNA
tgtagtggtggtgggggtaataacaatccatctcactctcctatggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaa

SEQ ID NO: 2 - protein, 318 aa, Pasteurella multocida
CSGGGGNNNPSHSPMEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQ

SEQ ID NO: 3 - DNA, 879 bp, Pasteurella multocida
  source 1..879: mol_type = genomic DNA
tgtagtggtggtgggggtggtaataatccatcacaatatcctaaagaagtacgccctgtggtaactgaacaagccaaaacagctaaagaagtgattttacctacaccctctattggatcagaacagtcatctcaaaacgtaatagctaaagatgctttcaatgtgagcgaaaaaacgcatcaacactccaaaccagaaaaaattgcaaaagatattaattggacaggtagtgctgtgtcgtctataggctctacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaatctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccag

SEQ ID NO: 4 - protein, 293 aa, Pasteurella multocida
CSGGGGGNNPSQYPKEVRPVVTEQAKTAKEVILPTPSIGSEQSSQNVIAKDAFNVSEKTHQHSKPEKIAKDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLIYREGKVSGEIFGQNNVKHFSFMNSNKIDPNSIVIVPEEAHRYLSRNDKMFLEMHFINGENGEKYKYIVGSGKTDKYYGALFATKQENQ

SEQ ID NO: 5 - DNA, 978 bp, Pasteurella multocida
  source 1..978: mol_type = genomic DNA
tgtagtggcggtgggggcggtaataacaatgtaccacaccctcctgtagaaaaacgtactgtggcgacaacacaacaagttgctgcaaaaccaactccgccttctgaaagtctagttaaacgagtactcaataattcagagaataacccaccatcaagagagtcagagaaaaaagcctcacaaactccaccacaaacagatagcgcgaaatcaacaatacaatctactcccgctgttataccagatcgtacaaactctttaacatctaagaaaggacaaattcaaatgtctccagaatggattgggaaagaagaaaaaaagtatgcgcaatattcatgggaacatacgccagaatctattccagtatttaaattaattgaaaataaccaatacaaatatgttgatgataagtactttacacttgaatcaatcaatcttaatttgactaaagaaaatcaagttaaagaaggttcttatcaatttagtctattagactctggagtctattatggccattacttaagctcaaacgatggtattcatccagaatataactttgtgattgcttttgacaaaaacagagaatacacactgaaagatatcacagctgaatactataattcggagggtttcaattatgctataagtgatagaatgaaaggtgactatatctggcaagtgggtgatgtgcgtttattctatacaaatggttctgtgcatggcgaaattgtagaagttaacgatggtagtaaaacagctctatttcgttttgaaaacacagcagatagaaacccaaatcaaattgttattgtcccagaaagagataatcgtcatggattatctcctcgaggagatcgcatgataatggatatgcattttattaatggtagtgatggagaaaaatacaaatatgtagttggtcacggtaattcagatcgatactacggtacactatttgcaaccaaaaaagataaagaa

SEQ ID NO: 6 - protein, 326 aa, Pasteurella multocida
CSGGGGGNNNVPHPPVEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKE

SEQ ID NO: 7 - DNA, 933 bp, Pasteurella multocida
  source 1..933: mol_type = genomic DNA
tgtagtggtggtgggggaggtggaagtaataacagcaatcatcaagctcatcctgttaatcaagtgcctgcgcccgtacttcatgtcgctgcaaaaccacaacaacatgttgaacaagaagttattgagaagaaacctccagttccagtgactagaacagcaagtcaatctagtttctattcagcaaaagcgccatcacaagtatctcacgataaaagatcggtacactggaaaggagaatcagtttctgaaaaagaacatcttgacttctcttacagtaaagatgccgtatttacccgtcatttggtaacaaatccaaacgctgtttatagcactgatcctaacctgattagtaaagacattaaatatattacattaaccactactggatataatcaagataataagagcggtccaaactatgagttaaatttgctcgatgaaaatatttattacggttactatcgtgattcacaagatatgaatcatgttgaaaatatctatgtatatgggttcaaaaaagatgcagaaaatcaagataatcttcaatttctaacagcaaattaccaaggagaatttttattctcaacggcaacaaacccaaatgtgccggtactaggtaaagctgtcttaaattacaaagaaggaaaagctaaaggcgaaattctagaacgagatagcaattacaaattatttgatatttatgttaatgagcgccctaatcaagcaattttgaatcctgtagctgaaagactacctacctctgatcttattatgaatacacgtaaaaattcaccagaccgtgtcacaatagatttacactttattaagggtcaagataaccaagaaaataaatatattgtaggtcaagggggtaatgaaaaatattggggcgtattaggtttagagaaaaaagagaccaaggcagaaaaa

SEQ ID NO: 8 - protein, 311 aa, Pasteurella multocida
CSGGGGGGSNNSNHQAHPVNQVPAPVLHVAAKPQQHVEQEVIEKKPPVPVTRTASQSSFYSAKAPSQVSHDKRSVHWKGESVSEKEHLDFSYSKDAVFTRHLVTNPNAVYSTDPNLISKDIKYITLTTTGYNQDNKSGPNYELNLLDENIYYGYYRDSQDMNHVENIYVYGFKKDAENQDNLQFLTANYQGEFLFSTATNPNVPVLGKAVLNYKEGKAKGEILERDSNYKLFDIYVNERPNQAILNPVAERLPTSDLIMNTRKNSPDRVTIDLHFIKGQDNQENKYIVGQGGNEKYWGVLGLEKKETKAEK

SEQ ID NO: 9 - DNA, 945 bp, Pasteurella multocida
  source 1..945: mol_type = genomic DNA
tgtagtggtggtgggggaggtggaagtaataacagcaatcatcaagctcatcctgttaatcaagtgcctgcgcccgtacttcatgttgctgcaaaaccacaacaacatgttgaagaaatccaagccgaagaaaaagttgttgagttagaacctctagtcaatgtagctcaaccaatcgaatcatcaaatttatccctaacagaatcacctaaaaaaccagcagacaataagatttcaaaacaatgggaaggggaatccattgccaaatggagaaaaacagactttaactatagtaaagacgccgtttttacacgtcacttagtggaaaatcccaaagctatttacgacacagacccaaatctgattagcaaagaaatccagtatattaaactacaactaggcgataacgttgataatgtggatgctccaaactatgaattaaatttactcaatgaaaatgcttactatggtttctatcgtgattcacaagatatgaaccatgttgaaaatatctatgtgtatggatttaaaaaagatgcagaaaaccatgaaaatcttcaatccttaaccgcaaattatgaaggagaatttttattttcaactgcgacaaatttaaatgtgactgttacaggaagagtcgtactcaaatatcaagagggtaaagctaaaggtgaaattctagaacgagatatcgattacaaattatttgatatatctgttgataagaaacctaatcaagctattcttaatcctgttgtagaggcattaccaggttcaaatataaaacttgtcaatcgtccaaattcaccagaccgtatcactgtagatttacactttattaagggtcaagataaccaagaaaataaatatattgtaggtcaagggggtaatgaaaaatattggggcgtattaggtttagagaaaaaagagaccagggcagaaaaa

SEQ ID NO: 10 - protein, 315 aa, Pasteurella multocida
CSGGGGGGSNNSNHQAHPVNQVPAPVLHVAAKPQQHVEEIQAEEKVVELEPLVNVAQPIESSNLSLTESPKKPADNKISKQWEGESIAKWRKTDFNYSKDAVFTRHLVENPKAIYDTDPNLISKEIQYIKLQLGDNVDNVDAPNYELNLLNENAYYGFYRDSQDMNHVENIYVYGFKKDAENHENLQSLTANYEGEFLFSTATNLNVTVTGRVVLKYQEGKAKGEILERDIDYKLFDISVDKKPNQAILNPVVEALPGSNIKLVNRPNSPDRITVDLHFIKGQDNQENKYIVGQGGNEKYWGVLGLEKKETRAEK

SEQ ID NO: 11 - protein, 975 aa, Pasteurella multocida
ATGGAAAAACGTACCGTAACTATACCACAAGCACAAAAAGTAGCAGAAAAACCAGCGCTAACAATACAAACAGTGGTAACTTCACCAACTACACCAATTAAAGCGCCCTCACTCGCTCCGACTATTGATACTGCGCCTAAACAAAATCCCGTGCCGACAGCGCAAATAACGGCTACTTTACCAGCCGAACCTGTTAAAGTGCCAGTTCTGATTCCCGAAGTAAACAAAACACTTTTAGAAACATCAACTAATCCAGTAAAAGATTCTTATAATAAAGATGCTGTATTTACATACGAATTAATCGCAAATCCAGATGCAGATTATAGCGATCAAAAATTGATTCTTAAAAAAGAAATTAGTTATATCAAACTTAATCTAGGCATAAATCAAGACAATAAAAATGCACCAAGCTATATTTTTAATTTACTAGATGACAATGTCTATTATGGTTTTTATCGTGATACTCAAGATATGAACAGAATAGAAAATAAATATACTTATGCTTTCAAAAAAGAAGCTGAAAATTTTGATAACCTTCAAAAATTTAATGCGACTTATGAAGGTCAATTTTGGTTCTCTAGTATAGATACTCCAAATGTACCTACTGTTGCAAGAGCATTCTTAACATATAACAATGGTAGAGTGGATGGTGAAATATTGGCTAAACATTGGAATGAAAAATTGTTTCAGATTACTGGATTTGATAATAATCCTCGTAAAGTAGAAATTTTTCCTACTGTAGAATATTTACCTAATTCGGGAACAAGACTAACAAAAGGAGCCACATCACCTCATCGTTTCCAAATGGACTTACATTTCATCAATAGTACAAATGGTGAAAAAAACAAATATCTTGTCGGCCAAGGTAGTACTGAGCAGTACTGGGGTGTTTTAGGTATGGAGAAAAAACAAGAGCTCGCTCTGGTGCCACGCGGTAGTTCCGCTCATCACCACCATCATCACCATCACCACCAC

SEQ ID NO: 12 - protein, 325 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQELALVPRGSSAHHHHHHHHHH

SEQ ID NO: 13 - DNA, 912 bp, Pasteurella multocida
  source 1..912: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaa

SEQ ID NO: 14 - protein, 304 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQ

SEQ ID NO: 15 - DNA, 639 bp, Pasteurella multocida
  source 1..639: mol_type = genomic DNA
ttgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaagagctcgctctggtgccacgcggtagttccgctcatcaccaccatcatcaccatcaccaccac

SEQ ID NO: 16 - protein, 213 aa, Pasteurella multocida
LILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQELALVPRGSSAHHHHHHHHHH

SEQ ID NO: 17 - DNA, 735 bp, Pasteurella multocida
  source 1..735: mol_type = genomic DNA
acatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggcggcagcacaagagctcgctctggtgccacgcggtagttccgctcatcaccaccatcatcaccatcaccaccac

SEQ ID NO: 18 - protein, 245 aa, Pasteurella multocida
TSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMAAAQELALVPRGSSAHHHHHHHHHH

SEQ ID NO: 19 - DNA, 933 bp, Pasteurella multocida
  source 1..933: mol_type = genomic DNA
atggaaaaacgtaccgttgcaaccacacagcaggttgcagcaaaaccgacaccgcctagcgaaagcctggttaaacgtgttctgaataacagcgaaaataatccgcctagccgtgaaagcgaaaaaaaagcaagtcagacccctccgcagaccgatagcgcaaaaagcaccattcagagcacaccggcagttattccggatcgtaccaatagcctgaccagcaaaaaaggtcagattcagatgagtccggaatggattggtaaagaagagaaaaaatacgcccagtatagctgggaacatacaccggaaagtattccggtttttaaactgatcgaaaacaaccagtacaagtacgtggatgacaaatatttcaccctggaaagcattaatctgaacctgacgaaagaaaaccaggtgaaagaaggtagctatcagtttagtctgctggatagcggtgtttattatggtcattatctgagcagcaacgatggtattcatcccgaatataactttgtgatcgccttcgataaaaaccgtgagtataccctgaaagatatcaccgcagaatattacaatagcgagggttttaactatgccattagcgatcgtatgaaaggcgattatatctggcaggttggtgatgttcgtctgttttataccaatggtagcgtgcatggtgaaattgtggaagttaatgatggtagtaaaaccgcactgtttcgctttgaaaataccgcagatcgtaatccgaatcagattgttattgtgccggaacgtgataatcgtcatggtctgagtccgcgtggtgatcgcatgattatggatatgcatttcattaatggcagcgacggcgagaaatacaaatatgttgttggtcatggtaacagcgatcgttattatggcaccctgtttgcaaccaaaaaagataaagag

SEQ ID NO: 20 - protein, 311 aa, Pasteurella multocida
MEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKE

SEQ ID NO: 21 - DNA, 954 bp, Pasteurella multocida; identical to SEQ ID NO: 1
  source 1..954: mol_type = genomic DNA

SEQ ID NO: 22 - protein, 318 aa, Pasteurella multocida; identical to SEQ ID NO: 2

SEQ ID NO: 23 - DNA, 978 bp, Pasteurella multocida
  source 1..978: mol_type = genomic DNA
tgtagtggcggtgggggcggtaataacaatgtaccacaccctcctgtagaaaaacgtactgtggcgacaacacaacaagttgctgcaaaaccaactccgccttctgaaagtctagttaaacgagtactcaataattcagagaataacccaccatcaagagagtcagagaaaaaagcctcacaaactccaccacaaacagatagcgcgaaatcaacaatacaatctactcccgctgttataccagatagtacaaactctttaacatctaagaaaggacaaattcaaatgtctccagaatggattgggaaagaagaaaaaaagtatgcgcaatattcatgggaacatacgccagaatctattccagtatttaaattaattgaaaataaccaatacaaatatgttaatgataagtactttacacttgaatcaatcaatcttaatttgactaaagaaaatcaagttaaagaaggttcttatcaatttagtctattagactctggagtctattatggccattacttaagctcaaacgatggtattcatccagaatataactttgtgattgcttttgacaaaaacagagaatacacactgaaagatatcacagctgaatactataattcggagggtttcaattatgctataagtgatagaatgaaaggtgactatatctggcaagtgggtgatgtgcgtttattctatacaaatggttctgtgcatggcgaaattgtagaagttaacgatggtagtaaaacagctctatttcgttttgaaaacacagcagatagaaacccaaatcaaattgttattgtcccagaaagagataatcgtcatggattatctcctcgaggagatcgcatgataatggatatgcattttattaatggtagtgatggagaaaaatacaaatatgtagttggtcacggtaattcagatcgatactacggtacactatttgcaaccaaaaaagataaagaa

SEQ ID NO: 24 - protein, 326 aa, Pasteurella multocida
CSGGGGGNNNVPHPPVEKRTVATTQQVAAKPTPPSESLVKRVLNNSENNPPSRESEKKASQTPPQTDSAKSTIQSTPAVIPDSTNSLTSKKGQIQMSPEWIGKEEKKYAQYSWEHTPESIPVFKLIENNQYKYVNDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKE

SEQ ID NO: 25 - DNA, 936 bp, Pasteurella multocida
  source 1..936: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaagactacaaggatgacgatgacaag

SEQ ID NO: 26 - protein, 312 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQDYKDDDDK

SEQ ID NO: 27 - DNA, 975 bp, Pasteurella multocida
  source 1..975: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaagagctcgctctggtgccacgcggtagttccgctcatcaccaccatcatcaccatcaccaccac

SEQ ID NO: 28 - protein, 325 aa, Pasteurella multocida; identical to SEQ ID NO: 12

SEQ ID NO: 29 - DNA, 996 bp, Pasteurella multocida
  source 1..996: mol_type = other DNA
atggaaaaacgtaccgttgcaaccacacagcaggttgcagcaaaaccgacaccgcctagcgaaagcctggttaaacgtgttctgaataacagcgaaaataatccgcctagccgtgaaagcgaaaaaaaagcaagtcagacccctccgcagaccgatagcgcaaaaagcaccattcagagcacaccggcagttattccggatcgtaccaatagcctgaccagcaaaaaaggtcagattcagatgagtccggaatggattggtaaagaagagaaaaaatacgcccagtatagctgggaacatacaccggaaagtattccggtttttaaactgatcgaaaacaaccagtacaagtacgtggatgacaaatatttcaccctggaaagcattaatctgaacctgacgaaagaaaaccaggtgaaagaaggtagctatcagtttagtctgctggatagcggtgtttattatggtcattatctgagcagcaacgatggtattcatcccgaatataactttgtgatcgccttcgataaaaaccgtgagtataccctgaaagatatcaccgcagaatattacaatagcgagggttttaactatgccattagcgatcgtatgaaaggcgattatatctggcaggttggtgatgttcgtctgttttataccaatggtagcgtgcatggtgaaattgtggaagttaatgatggtagtaaaaccgcactgtttcgctttgaaaataccgcagatcgtaatccgaatcagattgttattgtgccggaacgtgataatcgtcatggtctgagtccgcgtggtgatcgcatgattatggatatgcatttcattaatggcagcgacggcgagaaatacaaatatgttgttggtcatggtaacagcgatcgttattatggcaccctgtttgcaaccaaaaaagataaagaggagctcgctctggtgccacgcggtagttccgctcatcaccaccatcatcaccatcaccaccac

SEQ ID NO: 30 - protein, 332 aa, Pasteurella multocida
MEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKEELALVPRGSSAHHHHHHHHHH

SEQ ID NO: 31 - DNA, 1941 bp, Pasteurella multocida
  source 1..1941: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaagagctcgctctggtgccacgcggtagttccgctatggaaaaacgtaccgttgcaaccacacagcaggttgcagcaaaaccgacaccgcctagcgaaagcctggttaaacgtgttctgaataacagcgaaaataatccgcctagccgtgaaagcgaaaaaaaagcaagtcagacccctccgcagaccgatagcgcaaaaagcaccattcagagcacaccggcagttattccggatcgtaccaatagcctgaccagcaaaaaaggtcagattcagatgagtccggaatggattggtaaagaagagaaaaaatacgcccagtatagctgggaacatacaccggaaagtattccggtttttaaactgatcgaaaacaaccagtacaagtacgtggatgacaaatatttcaccctggaaagcattaatctgaacctgacgaaagaaaaccaggtgaaagaaggtagctatcagtttagtctgctggatagcggtgtttattatggtcattatctgagcagcaacgatggtattcatcccgaatataactttgtgatcgccttcgataaaaaccgtgagtataccctgaaagatatcaccgcagaatattacaatagcgagggttttaactatgccattagcgatcgtatgaaaggcgattatatctggcaggttggtgatgttcgtctgttttataccaatggtagcgtgcatggtgaaattgtggaagttaatgatggtagtaaaaccgcactgtttcgctttgaaaataccgcagatcgtaatccgaatcagattgttattgtgccggaacgtgataatcgtcatggtctgagtccgcgtggtgatcgcatgattatggatatgcatttcattaatggcagcgacggcgagaaatacaaatatgttgttggtcatggtaacagcgatcgttattatggcaccctgtttgcaaccaaaaaagataaagaggagctcgctctggtgccacgcggtagttccgctcatcaccaccatcatcaccatcaccaccac

SEQ ID NO: 32 - protein, 647 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQELALVPRGSSAMEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKEELALVPRGSSAHHHHHHHHHH

SEQ ID NO: 33 - DNA, 1914 bp, Pasteurella multocida
  source 1..1914: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaatccgctatggaaaaacgtaccgttgcaaccacacagcaggttgcagcaaaaccgacaccgcctagcgaaagcctggttaaacgtgttctgaataacagcgaaaataatccgcctagccgtgaaagcgaaaaaaaagcaagtcagacccctccgcagaccgatagcgcaaaaagcaccattcagagcacaccggcagttattccggatcgtaccaatagcctgaccagcaaaaaaggtcagattcagatgagtccggaatggattggtaaagaagagaaaaaatacgcccagtatagctgggaacatacaccggaaagtattccggtttttaaactgatcgaaaacaaccagtacaagtacgtggatgacaaatatttcaccctggaaagcattaatctgaacctgacgaaagaaaaccaggtgaaagaaggtagctatcagtttagtctgctggatagcggtgtttattatggtcattatctgagcagcaacgatggtattcatcccgaatataactttgtgatcgccttcgataaaaaccgtgagtataccctgaaagatatcaccgcagaatattacaatagcgagggttttaactatgccattagcgatcgtatgaaaggcgattatatctggcaggttggtgatgttcgtctgttttataccaatggtagcgtgcatggtgaaattgtggaagttaatgatggtagtaaaaccgcactgtttcgctttgaaaataccgcagatcgtaatccgaatcagattgttattgtgccggaacgtgataatcgtcatggtctgagtccgcgtggtgatcgcatgattatggatatgcatttcattaatggcagcgacggcgagaaatacaaatatgttgttggtcatggtaacagcgatcgttattatggcaccctgtttgcaaccaaaaaagataaagaggagctcgctctggtgccacgcggtagttccgctcatcaccaccatcatcaccatcaccaccac

SEQ ID NO: 34 - protein, 638 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQSAMEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKEELALVPRGSSAHHHHHHHHHH

SEQ ID NO: 35 - DNA, 2016 bp, Pasteurella multocida
  source 1..2016: mol_type = genomic DNA
atgcagtattttgacatcaaaaaatctctccctgttttttgttctcttctgattaccgcttgtagtggtggtgggggtaataacaatccatctcactctcctatggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaatccgctatggaaaaacgtaccgttgcaaccacacagcaggttgcagcaaaaccgacaccgcctagcgaaagcctggttaaacgtgttctgaataacagcgaaaataatccgcctagccgtgaaagcgaaaaaaaagcaagtcagacccctccgcagaccgatagcgcaaaaagcaccattcagagcacaccggcagttattccggatcgtaccaatagcctgaccagcaaaaaaggtcagattcagatgagtccggaatggattggtaaagaagagaaaaaatacgcccagtatagctgggaacatacaccggaaagtattccggtttttaaactgatcgaaaacaaccagtacaagtacgtggatgacaaatatttcaccctggaaagcattaatctgaacctgacgaaagaaaaccaggtgaaagaaggtagctatcagtttagtctgctggatagcggtgtttattatggtcattatctgagcagcaacgatggtattcatcccgaatataactttgtgatcgccttcgataaaaaccgtgagtataccctgaaagatatcaccgcagaatattacaatagcgagggttttaactatgccattagcgatcgtatgaaaggcgattatatctggcaggttggtgatgttcgtctgttttataccaatggtagcgtgcatggtgaaattgtggaagttaatgatggtagtaaaaccgcactgtttcgctttgaaaataccgcagatcgtaatccgaatcagattgttattgtgccggaacgtgataatcgtcatggtctgagtccgcgtggtgatcgcatgattatggatatgcatttcattaatggcagcgacggcgagaaatacaaatatgttgttggtcatggtaacagcgatcgttattatggcaccctgtttgcaaccaaaaaagataaagaggagctcgctctggtgccacgcggtagttccgctcatcaccaccatcatcaccatcaccaccac

SEQ ID NO: 36 - protein, 672 aa, Pasteurella multocida
MQYFDIKKSLPVFCSLLITACSGGGGNNNPSHSPMEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQSAMEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKEELALVPRGSSAHHHHHHHHHH

SEQ ID NO: 37 - DNA, 705 bp, Pasteurella multocida
  source 1..705: mol_type = genomic DNA
tgtagtggtggtgggggtaataacaatccatctcactctcctatggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggaa

SEQ ID NO: 38 - protein, 235 aa, Pasteurella multocida
CSGGGGNNNPSHSPMEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILE

SEQ ID NO: 39 - DNA, 945 bp, Pasteurella multocida
  source 1..945: mol_type = genomic DNA
tgtagtggtggtgggggaggtggaagtaataacagcaatcatcaagctcatcctgttaatcaagtgcctgcgcccgtacttcatgttgctgcaaaaccacaacaacatgttgaagaaatccaagtcgaagaaaaagttgttgagttagaacctctagtcaatgtagctcaaccaatcgaatcatcaaatttatccctaacagaatcacctaaaaaaccagcagacaataagatttcaaaacaatgggaaggggaatccattgccaaatggagaaaaacagactttaactatagtaaagacgccgtttttacacgtcacttagtggaaaatcccaaagctatttacgacacagacccaaatctgattagcaaagaaatccagtatattaaactacaactaggcgataacgttgataatgtggatgctccaaactatgaattaaatttactcaatgaaaatgcttactatggtttctatcgtgattcacaagatatgaaccatgttgaaaatatctatgtgtatggatttaaaaaagatgcagaaaaccatgaaaatcttcaatccttaaccgcaaattatgaaggagaatttttattttcaactgcgacaaatttaaatgtgactgttacaggaagagtcgtactcaaatatcaagagggtaaagctaaaggtgaaattctagaacgagatatcgattacaaattatttgatatatctgttgataagaaacctaatcaagctattcttaatcctgttgtagaggcattaccaggttcaaatataaaacttgtcaatcgtccaaattcaccagaccgtatcactgtagatttacactttattaagggtcaagataaccaagaaaataaatatattgtaggtcaagggggtaatgaaaaatattggggcgtattaggtctagagaaaaaagagaccaaggcagaaaaa

SEQ ID NO: 40 - protein, 315 aa, Pasteurella multocida
CSGGGGGGSNNSNHQAHPVNQVPAPVLHVAAKPQQHVEEIQVEEKVVELEPLVNVAQPIESSNLSLTESPKKPADNKISKQWEGESIAKWRKTDFNYSKDAVFTRHLVENPKAIYDTDPNLISKEIQYIKLQLGDNVDNVDAPNYELNLLNENAYYGFYRDSQDMNHVENIYVYGFKKDAENHENLQSLTANYEGEFLFSTATNLNVTVTGRVVLKYQEGKAKGEILERDIDYKLFDISVDKKPNQAILNPVVEALPGSNIKLVNRPNSPDRITVDLHFIKGQDNQENKYIVGQGGNEKYWGVLGLEKKETKAEK

SEQ ID NO: 41 - protein, 11 aa, synthetic construct
ELALVPRGSSA

SEQ ID NO: 42 - protein, 20 aa, synthetic construct
MQYFDIKKSLPVFCSLLITA

SEQ ID NO: 43 - protein, 14 aa, synthetic construct
VLGMEKKQSAMEKR

SEQ ID NO: 44 - DNA, 50 bp, synthetic construct
  source 1..50: mol_type = other DNA
taactttaagaaggagatatacatatggaaaaacgtaccgtaactatacc

SEQ ID NO: 45 - DNA, 50 bp, synthetic construct
  source 1..50: mol_type = other DNA
taactttaagaaggagatatacatatgacatcaactaatccagtaaaaga

SEQ ID NO: 46 - DNA, 48 bp, synthetic construct
  source 1..48: mol_type = other DNA
taactttaagaaggagatatacatatggattatagcgatcaaaaattg

SEQ ID NO: 47 - DNA, 53 bp, synthetic construct
  source 1..53: mol_type = other DNA
actaccgcgtggcaccagagcgagctcttgttttttctccatacctaaaacac

SEQ ID NO: 48 - DNA, 50 bp, synthetic construct
  source 1..50: mol_type = other DNA
ggtgttttaggtatggcggcagcacaagagctcgctctggtgccacgcgg

SEQ ID NO: 49 - DNA, 52 bp, synthetic construct
  source 1..52: mol_type = other DNA
caccagagcgagctcttgtgctgccgccatacctaaaacaccccagtactgc

SEQ ID NO: 50 - DNA, 1818 bp, Pasteurella multocida
  source 1..1818: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaatccgctatggaagtacgccctgtggtaactgaacaagccaaaacagctaaagaagtgattttacctacaccctctattggatcagaacagtcatctcaaaacgtaatagctaaagatgctttcaatgtgagcgaaaaaacgcatcaacactccaaaccagaaaaaattgcaaaagatattaattggacaggtagtgctgtgtcgtctataggctctacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaatctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccaggagctcgctctggtgccacgcggtagttccgctcatcaccaccatcatcaccatcaccaccac

SEQ ID NO: 51 - protein, 606 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQSAMEVRPVVTEQAKTAKEVILPTPSIGSEQSSQNVIAKDAFNVSEKTHQHSKPEKIAKDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLIYREGKVSGEIFGQNNVKHFSFMNSNKIDPNSIVIVPEEAHRYLSRNDKMFLEMHFINGENGEKYKYIVGSGKTDKYYGALFATKQENQELALVPRGSSAHHHHHHHHHH

SEQ ID NO: 52 - DNA, 2757 bp, Pasteurella multocida
  source 1..2757: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaatccgctatggaagtacgccctgtggtaactgaacaagccaaaacagctaaagaagtgattttacctacaccctctattggatcagaacagtcatctcaaaacgtaatagctaaagatgctttcaatgtgagcgaaaaaacgcatcaacactccaaaccagaaaaaattgcaaaagatattaattggacaggtagtgctgtgtcgtctataggctctacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaatctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccagtccgctatggaaaaacgtaccgttgcaaccacacagcaggttgcagcaaaaccgacaccgcctagcgaaagcctggttaaacgtgttctgaataacagcgaaaataatccgcctagccgtgaaagcgaaaaaaaagcaagtcagacccctccgcagaccgatagcgcaaaaagcaccattcagagcacaccggcagttattccggatcgtaccaatagcctgaccagcaaaaaaggtcagattcagatgagtccggaatggattggtaaagaagagaaaaaatacgcccagtatagctgggaacatacaccggaaagtattccggtttttaaactgatcgaaaacaaccagtacaagtacgtggatgacaaatatttcaccctggaaagcattaatctgaacctgacgaaagaaaaccaggtgaaagaaggtagctatcagtttagtctgctggatagcggtgtttattatggtcattatctgagcagcaacgatggtattcatcccgaatataactttgtgatcgccttcgataaaaaccgtgagtataccctgaaagatatcaccgcagaatattacaatagcgagggttttaactatgccattagcgatcgtatgaaaggcgattatatctggcaggttggtgatgttcgtctgttttataccaatggtagcgtgcatggtgaaattgtggaagttaatgatggtagtaaaaccgcactgtttcgctttgaaaataccgcagatcgtaatccgaatcagattgttattgtgccggaacgtgataatcgtcatggtctgagtccgcgtggtgatcgcatgattatggatatgcatttcattaatggcagcgacggcgagaaatacaaatatgttgttggtcatggtaacagcgatcgttattatggcaccctgtttgcaaccaaaaaagataaagaggagctcgctctggtgccacgcggtagttccgctcatcaccaccatcatcaccatcaccaccac

SEQ ID NO: 53 - protein, 919 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQSAMEVRPVVTEQAKTAKEVILPTPSIGSEQSSQNVIAKDAFNVSEKTHQHSKPEKIAKDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLIYREGKVSGEIFGQNNVKHFSFMNSNKIDPNSIVIVPEEAHRYLSRNDKMFLEMHFINGENGEKYKYIVGSGKTDKYYGALFATKQENQSAMEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKEELALVPRGSSAHHHHHHHHHH

SEQ ID NO: 54 - DNA, 2646 bp, Pasteurella multocida
  source 1..2646: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaatccgctatggaagtacgccctgtggtaactgaacaagccaaaacagctaaagaagtgattttacctacaccctctattggatcagaacagtcatctcaaaacgtaatagctaaagatgctttcaatgtgagcgaaaaaacgcatcaacactccaaaccagaaaaaattgcaaaagatattaattggacaggtagtgctgtgtcgtctataggctctacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaatctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccagtccgctatggaagaaaaagttgttgagttagaacctctagtcaatgtagctcaaccaatcgaatcatcaaatttatccctaacagaatcacctaaaaaaccagcagacaataagatttcaaaacaatgggaaggggaatccattgccaaatggagaaaaacagactttaactatagtaaagacgccgtttttacacgtcacttagtggaaaatcccaaagctatttacgacacagacccaaatctgattagcaaagaaatccagtatattaaactacaactaggcgataacgttgataatgtggatgctccaaactatgaattaaatttactcaatgaaaatgcttactatggtttctatcgtgattcacaagatatgaaccatgttgaaaatatctatgtgtatggatttaaaaaagatgcagaaaaccatgaaaatcttcaatccttaaccgcaaattatgaaggagaatttttattttcaactgcgacaaatttaaatgtgactgttacaggaagagtcgtactcaaatatcaagagggtaaagctaaaggtgaaattctagaacgagatatcgattacaaattatttgatatatctgttgataagaaacctaatcaagctattcttaatcctgttgtagaggcattaccaggttcaaatataaaacttgtcaatcgtccaaattcaccagaccgtatcactgtagatttacactttattaagggtcaagataaccaagaaaataaatatattgtaggtcaagggggtaatgaaaaatattggggcgtattaggtttagagaaaaaagagaccagggcagaaaaagagctcgctctggtgccacgcggtagttccgctcatcaccaccatcatcaccatcaccaccac

SEQ ID NO: 55 - protein, 882 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQSAMEVRPVVTEQAKTAKEVILPTPSIGSEQSSQNVIAKDAFNVSEKTHQHSKPEKIAKDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLIYREGKVSGEIFGQNNVKHFSFMNSNKIDPNSIVIVPEEAHRYLSRNDKMFLEMHFINGENGEKYKYIVGSGKTDKYYGALFATKQENQSAMEEKVVELEPLVNVAQPIESSNLSLTESPKKPADNKISKQWEGESIAKWRKTDFNYSKDAVFTRHLVENPKAIYDTDPNLISKEIQYIKLQLGDNVDNVDAPNYELNLLNENAYYGFYRDSQDMNHVENIYVYGFKKDAENHENLQSLTANYEGEFLFSTATNLNVTVTGRVVLKYQEGKAKGEILERDIDYKLFDISVDKKPNQAILNPVVEALPGSNIKLVNRPNSPDRITVDLHFIKGQDNQENKYIVGQGGNEKYWGVLGLEKKETRAEKELALVPRGSSAHHHHHHHHHH

SEQ ID NO: 56 - DNA, 900 bp, Pasteurella multocida
  source 1..900: mol_type = genomic DNA
atggaagtacgccctgtggtaactgaacaagccaaaacagctaaagaagtgattttacctacaccctctattggatcagaacagtcatctcaaaacgtaatagctaaagatgctttcaatgtgagcgaaaaaacgcatcaacactccaaaccagaaaaaattgcaaaagatattaattggacaggtagtgctgtgtcgtctataggctctacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaatctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccaggagctcgctctggtgccacgcggtagttccgctcatcaccaccatcatcaccatcaccaccac

SEQ ID NO: 57 - protein, 300 aa, Pasteurella multocida
MEVRPVVTEQAKTAKEVILPTPSIGSEQSSQNVIAKDAFNVSEKTHQHSKPEKIAKDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLIYREGKVSGEIFGQNNVKHFSFMNSNKIDPNSIVIVPEEAHRYLSRNDKMFLEMHFINGENGEKYKYIVGSGKTDKYYGALFATKQENQELALVPRGSSAHHHHHHHHHH

SEQ ID NO: 58 - DNA, 837 bp, Pasteurella multocida
  source 1..837: mol_type = genomic DNA
atggaagtacgccctgtggtaactgaacaagccaaaacagctaaagaagtgattttacctacaccctctattggatcagaacagtcatctcaaaacgtaatagctaaagatgctttcaatgtgagcgaaaaaacgcatcaacactccaaaccagaaaaaattgcaaaagatattaattggacaggtagtgctgtgtcgtctataggctctacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaatctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccag

SEQ ID NO: 59 - protein, 279 aa, Pasteurella multocida
MEVRPVVTEQAKTAKEVILPTPSIGSEQSSQNVIAKDAFNVSEKTHQHSKPEKIAKDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLIYREGKVSGEIFGQNNVKHFSFMNSNKIDPNSIVIVPEEAHRYLSRNDKMFLEMHFINGENGEKYKYIVGSGKTDKYYGALFATKQENQ

SEQ ID NO: 60 - DNA, 900 bp, Pasteurella multocida
  source 1..900: mol_type = genomic DNA
gctcatcctgttaatcaagtgcctgcgcccgtacttcatgttgctgcaaaaccacaacaacatgttgaagaaatccaagccgaagaaaaagttgttgagttagaacctctagtcaatgtagctcaaccaatcgaatcatcaaatttatccctaacagaatcacctaaaaaaccagcagacaataagatttcaaaacaatgggaaggggaatccattgccaaatggagaaaaacagactttaactatagtaaagacgccgtttttacacgtcacttagtggaaaatcccaaagctatttacgacacagacccaaatctgattagcaaagaaatccagtatattaaactacaactaggcgataacgttgataatgtggatgctccaaactatgaattaaatttactcaatgaaaatgcttactatggtttctatcgtgattcacaagatatgaaccatgttgaaaatatctatgtgtatggatttaaaaaagatgcagaaaaccatgaaaatcttcaatccttaaccgcaaattatgaaggagaatttttattttcaactgcgacaaatttaaatgtgactgttacaggaagagtcgtactcaaatatcaagagggtaaagctaaaggtgaaattctagaacgagatatcgattacaaattatttgatatatctgttgataagaaacctaatcaagctattcttaatcctgttgtagaggcattaccaggttcaaatataaaacttgtcaatcgtccaaattcaccagaccgtatcacggtagatttacactttattaagggtcaagataaccaagaaaataaatatattgtaggtcaagggggtaatgaaaaatattggggcgtattaggtttagagaaaaaagagaccaaggcagaaaaa

SEQ ID NO: 61 - protein, 300 aa, Pasteurella multocida
AHPVNQVPAPVLHVAAKPQQHVEEIQAEEKVVELEPLVNVAQPIESSNLSLTESPKKPADNKISKQWEGESIAKWRKTDFNYSKDAVFTRHLVENPKAIYDTDPNLISKEIQYIKLQLGDNVDNVDAPNYELNLLNENAYYGFYRDSQDMNHVENIYVYGFKKDAENHENLQSLTANYEGEFLFSTATNLNVTVTGRVVLKYQEGKAKGEILERDIDYKLFDISVDKKPNQAILNPVVEALPGSNIKLVNRPNSPDRITVDLHFIKGQDNQENKYIVGQGGNEKYWGVLGLEKKETKAEK

SEQ ID NO: 62 - DNA, 888 bp, Pasteurella multocida
  source 1..888: mol_type = genomic DNA
gctcatcctgttaatcaagtgcctgcgcccgtacttcatgtcgctgcaaaaccacaacaacatgttgaacaagaagttattgagaagaaacctccagttcgagtgactagaacagcaagtcaatctagtttctattcagcaaaagcgccatcacaagtatctcacgataaaagatcggtacactggaaaggagaatcagtttctgaaaaagaacatcttgacttctcttacagtaaagatgccgtatttacccgtcatttggtaacaaatccaaacgctgtttatagcactgatcctaacctgattagtaaagacattaaatatattacattaaccactactggatataatcaagataataagagcggtccaaactatgagttaaatttgctcgatgaaaatatttattacggttactatcgtgattcacaagatatgaatcatgttgaaaatatctatgtatatgggttcaaaaaagatgcagaaaatcaagataatcttcaatttctaacagcaaattaccaaggagaatttttattctcaacggcaacaaacccaaatgtgccggtactaggtaaagctgtcttaaattacaaagaaggaaaagctaaaggcgaaattctagaacgagatagcaattacaaattatttgatatttatgttaatgagcgccctaatcaagcaattttgaatcctgtagctgaaagactacctacctctgatcttattatgaatacacgtaaaaattcaccagaccgtgtcacaatagatttacactttattaagggtcaagataaccaagaaaataaatatattgtaggtcaagggggtaatgaaaaatattggggcgtattaggtttagagaaaaaagagaccaaggcagaaaaa

SEQ ID NO: 63 - protein, 296 aa, Pasteurella multocida
AHPVNQVPAPVLHVAAKPQQHVEQEVIEKKPPVRVTRTASQSSFYSAKAPSQVSHDKRSVHWKGESVSEKEHLDFSYSKDAVFTRHLVTNPNAVYSTDPNLISKDIKYITLTTTGYNQDNKSGPNYELNLLDENIYYGYYRDSQDMNHVENIYVYGFKKDAENQDNLQFLTANYQGEFLFSTATNPNVPVLGKAVLNYKEGKAKGEILERDSNYKLFDIYVNERPNQAILNPVAERLPTSDLIMNTRKNSPDRVTIDLHFIKGQDNQENKYIVGQGGNEKYWGVLGLEKKETKAEK

SEQ ID NO: 64 - DNA, 975 bp, Pasteurella multocida
  source 1..975: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgatcctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaagagctcgctctggtgccacgcggtagttccgctcatcaccaccatcatcaccatcaccaccac

SEQ ID NO: 65 - protein, 325 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNDPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQELALVPRGSSAHHHHHHHHHH

SEQ ID NO: 66 - DNA, 975 bp, Pasteurella multocida
  source 1..975: mol_type = genomic DNA
atggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgcaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaagagctcgctctggtgccacgcggtagttccgctcatcaccaccatcatcaccatcaccaccac

SEQ ID NO: 67 - protein, 325 aa, Pasteurella multocida
MEKRTVTIPQAQKVAEKPALTIQTVVTSPTTPIKAPSLAPTIDTAPKQNPVPTAQITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNAKLFQITGFDNNPRKVEIFPTVEYLPNSGTRLTKGATSPHRFQMDLHFINSTNGEKNKYLVGQGSTEQYWGVLGMEKKQELALVPRGSSAHHHHHHHHHH

SEQ ID NO: 68 - DNA, 966 bp, Pasteurella multocida
  source 1..966: mol_type = genomic DNA
tgtagtggtgggagtggtggaagtaataacagcaatcatcaagctcatcctgttaatcaagtgcgtactccaatgcctgctaccgtagcaaaacctgaattacccaaaccagtagcacttattgaagaaacagtgcaagctgaagaaaaagtcgtggaagagaaacctttagttgccactgcgactattcaaccagcagttcaatcaaatatctattccgaaaaagcaccaacacaaacatctgataataaaaaatcagtaaactggcaaggagagtctatttcagaaaagggaaatcgcgacttctcttacaataaaaatgctgtatttactcgtcatttagtaacaaatccaaatgccgtttatgacacagatcctaacctgattagtaaagatattaaatatattacattaacggatggatacaaccaagataataagaatggtccaaactatgagttaaatttgctcgatgaaaatgtttattatggctactatcgtgattcacaagatatgaaccatgttgaaaatatctatgtatacgggttcaaaaaagatgcagaaaatcaagatactctccaatttttaacagcaaattatcaaggtgagtttttattttcaacagcgacaaacccaaatgtgcctatattaggtaaagctatcttaaattacaaagaaggaaaagctaaaggtgaaattctagaacgagataacaattacaaattatttgatatttatgttaatgaacgccctaatcaagctattctaagtcctgtagctgaaagattacccgcttctgatattattatggatacacgtaaaaactcatcagaccgaatctcaatagatctgcacttcatcaaaagtaaagataatcaagaaaataaatatatcgtaggtcaaggtggtaacgaaaaatactggggcgttttaggtcttgagaaaaaagatacaccaactgcaaaa

SEQ ID NO: 69 - protein, 322 aa, Pasteurella multocida
CSGGSGGSNNSNHQAHPVNQVRTPMPATVAKPELPKPVALIEETVQAEEKVVEEKPLVATATIQPAVQSNIYSEKAPTQTSDNKKSVNWQGESISEKGNRDFSYNKNAVFTRHLVTNPNAVYDTDPNLISKDIKYITLTDGYNQDNKNGPNYELNLLDENVYYGYYRDSQDMNHVENIYVYGFKKDAENQDTLQFLTANYQGEFLFSTATNPNVPILGKAILNYKEGKAKGEILERDNNYKLFDIYVNERPNQAILSPVAERLPASDIIMDTRKNSSDRISIDLHFIKSKDNQENKYIVGQGGNEKYWGVLGLEKKDTPTAK

SEQ ID NO: 70 - DNA, 954 bp, Pasteurella multocida
  source 1..954: mol_type = genomic DNA
tgtagtggtggtgggggtaataacaatccatctcactctcctatggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgcaaataacggctactttcccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaa

SEQ ID NO: 71 - protein, 318 aa, Pasteurella multocida
CSGGGGNNNPSHSPMEKRTVTIPQAQKVAEKPALTIQTVVTSPTTPIKAPSLAPTIDTAPKQNPVPTAQITATFPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQ

SEQ ID NO: 72 - DNA, 954 bp, Pasteurella multocida
  source 1..954: mol_type = genomic DNA
tgtagtggtggtgggggtaataacaatccatctcactctcctatggaaaaacgtaccgtaactataccacaagcacaaaaagtagcagaaaaaccagcgctaacaatacaaacagtggtaacttcaccaactacaccaattaaagcgccctcactcgctccgactattgatactgcgcctaaacaaaatcccgtgccgacagcgaaaataacggctactttaccagccgaacctgttaaagtgccagttctgattcccgaagtaaacaaaacacttttagaaacatcaactaatccagtaaaagattcttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaa

SEQ ID NO: 73 - protein, 318 aa, Pasteurella multocida
CSGGGGNNNPSHSPMEKRTVTIPQAQKVAEKPALTIQTVVTSPTTPIKAPSLAPTIDTAPKQNPVPTAKITATLPAEPVKVPVLIPEVNKTLLETSTNPVKDSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQ

SEQ ID NO: 74 - DNA, 933 bp, Pasteurella multocida
  source 1..933: mol_type = genomic DNA
tgtagtggtggtgggggaggtggaagtaataacagcaatcatcaagctcatcctgttaatcaagtgcctgcgcccgtacttcatgttgctgcaaaaccacaacaacatgttgaacaagaagttattgagaagaaacctccagttcgagtgactagaacagcaagtcaatctagtttctattcagcaaaagcgccatcacaagtatctcacgataaaagatcggtacactggaaaggagaatcagtttctgaaaaagaacatcttgacttctcttacagtaaagatgccgtatttacccgtcatttggtaacaaatccaaacgctgtttatagcactgatcctaacctgattagtaaagacattaaatatattacattaaccactactggatataatcaagataataagagcggtccaaactatgagttaaatttgctcgatgaaaatatttattacggttactatcgtgattcacaagatatgaatcatgttgaaaatatctatgtatatgggttcaaaaaagatgcagaaaatcaagataatcttcaatttctaacagcaaattaccaaggagaatttttattctcaacggcaacaaacccaaatgtgccggtactaggtaaagctgtcttaaattacaaagaaggaaaagctaaaggcgaaattctagaacgagatagcaattacaaattatttgatatttatgttaatgagcgccctaatcaagcaattttgaatcctgtagctgaaagactacctacctctgatcttattatgaatacacgtaaaaattcaccagaccgtgtcacaatagatttacactttattaagggtcaagataaccaagaaaataaatatattgtaggtcaagggggtaatgaaaaatattggggcgtattaggtttagagaaaaaagagaccaaggcagaaaaa

SEQ ID NO: 75 - protein, 311 aa, Pasteurella multocida
CSGGGGGGSNNSNHQAHPVNQVPAPVLHVAAKPQQHVEQEVIEKKPPVRVTRTASQSSFYSAKAPSQVSHDKRSVHWKGESVSEKEHLDFSYSKDAVFTRHLVTNPNAVYSTDPNLISKDIKYITLTTTGYNQDNKSGPNYELNLLDENIYYGYYRDSQDMNHVENIYVYGFKKDAENQDNLQFLTANYQGEFLFSTATNPNVPVLGKAVLNYKEGKAKGEILERDSNYKLFDIYVNERPNQAILNPVAERLPTSDLIMNTRKNSPDRVTIDLHFIKGQDNQENKYIVGQGGNEKYWGVLGLEKKETKAEK

SEQ ID NO: 76 - DNA, 945 bp, Pasteurella multocida
  source 1..945: mol_type = genomic DNA
tgtagtggtggtgggggaggtggaagtaataacagcaatcatcaagctcatcctgttaatcaagtgcctgcgcccgtacttcatgttgctgcaaaaccacaacaacatgttgaagaaatccaagtcgaagaaaaagttgttgagttagaacctctagtcaatgtagctcaaccaatcgaatcatcaaatttatccctaacagaatcacctaaaaaaccagcagacaataagatttcaaaacaatgggaaggggaatccattgccaaatggagaaaaacagactttaactatagtaaagacgccgtttttacacgtcacttagtggaaaatcccaaagctatttacgacacagacccaaatctgattagcaaagaaatccagtatattaaactacaactaggcgataacgttgataatgtggatgctccaaactatgaattaaatttactcaatgaaaatgcttactatggtttctatcgtgattcacaagatatgaaccatgttgaaaatatctatgtgtatggatttaaaaaagatgcagaaaaccatgaaaatcttcaatccttaaccgcaaattatgaaggagaatttttattttcaactgcgacaaatttaaatgtgactgttacaggaagagtcgtactcaaatatcaagagggtaaagctaaaggtgaaattctagaacgagatatcgattacaaattatttgatatatctgttgataagaaacctaatcaagctattcttaatcctgttgtagaggcattaccaggttcaaatataaaacttgtcaatcgtccaaattcaccagaccgtatcactgtagatttacactttattaagggtcaagataaccaagaaaataaatatattgtaggtcaagggggtaatgaaaaatattggggcgtattaggtttagagaaaaaagagaccaaggcagaaaaa

SEQ ID NO: 77 - protein, 315 aa, Pasteurella multocida; identical to SEQ ID NO: 40

SEQ ID NO: 78 - DNA, 978 bp, Pasteurella multocida
  source 1..978: mol_type = genomic DNA
tgtagtggcggtgggggcggtaataacaatgtaccacaccctcctgtagaaaaacgtactgtggcgacaacacaacaagttgctgcaaaaccaactccgccttctgaaagtctagttaaacgagtactcaataattcagagaataacccaccatcaagagagtcagagaaaaaagcctcacaaactccaccacaaacagatagcgcgaaatcaacaatacaatctactcccgctgttataccagatagtacaaactctttaacatctaagaaaggacaaattcaaatgtctccagaatggattgggaaagaagaaaaaaagtatgcgcaatattcatgggaacatacgccagaatctattccagtatttaaattaattgaaaataaccaatacaaatatgttaatgataagtactttacacttgaatcaatcaatcttaatttgactaaagaaaatcaagttaaagaaggttcttatcaatttagtctattagactctggagtctattatggccattacttaagctcaaacgatggtattcatccagaatataactttgtgattgcttttgacaaaaacagagaatacacactgaaagatatcacagctgaatactataattcggagggtttcaattatgctataagtgatagaatgaaaggtgactatatctggcaagtgggtgatgtgcgtttattctatacaaatggttctgtgcatggcgaaattgtagaagttaacgatggtagtaaaacagctctatttcgttttgaaaacacagcagatagaaacccaaatcaaattgttattgtcccagaaagaaataatcgtcatggattatctcctcgaggagatcgcatgataatggatatgcattttattaatggtagtgatggagaaaaatacaaatatgtagttggtcacggtaattcagatcgatactacggtacactatttgcaaccaaaaaagataaagaa

SEQ ID NO: 79 - protein, 326 aa, Pasteurella multocida
CSGGGGGNNNVPHPPVEKRTVATTQQVAAKPTPPSESLVKRVLNNSENNPPSRESEKKASQTPPQTDSAKSTIQSTPAVIPDSTNSLTSKKGQIQMSPEWIGKEEKKYAQYSWEHTPESIPVFKLIENNQYKYVNDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERNNRHGLSPRGDRMIMDMHFINGSDGEKYKYVVGHGNSDRYYGTLFATKKDKE

SEQ ID NO: 80 - DNA, 978 bp, Pasteurella multocida; identical to SEQ ID NO: 23
  source 1..978: mol_type = genomic DNA

SEQ ID NO: 81 - protein, 326 aa, Pasteurella multocida; identical to SEQ ID NO: 24

SEQ ID NO: 82 - DNA, 978 bp, Pasteurella multocida
  source 1..978: mol_type = genomic DNA
tgtagtggcggtgggggcggtaataacaatgtaccacaccctcctgtagaaaaacgtactgtggcgacaacacaacaagttgctgcaaaaccaactccgccttctgaaagtctagttaaacgagtactcaataattcagagaataacccaccatcaagagagtcagagaaaaaagcctcacaaactccaccacaaacagatagcgcgaaatcaacaatacaatctactcccgctgttataccagatagtacaaactctttaacatctaagaaaggacaaattcaaatgtctccagaatggattgggaaagaagaaaaaaagtatgcgcaatattcatgggaacatacgccagaatctattccagtatttaaattaattgaaaataaccaatacaaatatgttaatgataagtactttacacttgaatcaatcaatcttaatttgactaaagaaaatcaagttaaagaaggttcttatcaatttagtctattagactctggagtctattatggccattacttaagctcaaacgatggtattcatccagaatataactttgtgattgcttttgacaaaaacagagaatacacactgaaagatatcacagctgaatactataattcggagggtttcaattatgctataagtgatagaatgaaaggtgactatatctggcaagtgggtgatgtgcgtttattctatacaaatggttctgtgcatggcgaaattgtagaagttaaggatggtagtaaaacagctctatttcgttttgaaaacacagcagatagaaacccaaatcaaattgttattgtcccagaaagagataatcgtcatggattatctcctcgaggagatcgcatgataatggatatgcattttattaatggtagtgatggagaaaaatacaaatatgtagttggtcacggtaattcagatcgatactacggtacactatttgcaaccaaaaaagataaagaa

SEQ ID NO: 83 - protein, 326 aa, Pasteurella multocida
CSGGGGGNNNVPHPPVEKRTVATTQQVAAKPTPPSESLVKRVLNNSENNPPSRESEKKASQTPPQTDSAKSTIQSTPAVIPDSTNSLTSKKGQIQMSPEWIGKEEKKYAQYSWEHTPESIPVFKLIENNQYKYVNDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVKDGSKTALFRFENTADRNPNQIVIVPERDNRHGLSPRGDRMIMDMHFINGSDGEKYKYVVGHGNSDRYYGTLFATKKDKE

SEQ ID NO: 84 - DNA, 978 bp, Pasteurella multocida
  source 1..978: mol_type = genomic DNA
tgtagtggcggtgggggcggtaataacaatgtaccacaccctcctgtagaaaaacgtactgtggcgacaacacaacaagttgctgcaaaaccaactccgccttctgaaagtctagttaaacgagtactcaataattcagagaataacccaccatcaagagagtcagagaaaaaagcctcacaaactccaccacaagcagatagcgcgaaatcaacaatacaatctactcccgctgttataccagatagtacaaactctttaacatctaagaaaggacaaattcaaatgtctccagaatggattgggaaagaagaaaaaaagtatgcgcaatattcatgggaacatacgccagaatctattccagtatttaaattaattgaaaataaccaatacaaatatgttaatgataagtactttacacttgaaccaatcaatcttaatttgactaaagaaaatcaagttaaagaaggttcttatcaatttagtctattagactctggagtctattatggccattacttaagctcaaacgatggtattcatccagaatataactttgtgattgcttttgacaaaaacagagaatacacactgaaagatatcacagctgaatactataattcggagggtttcaattatgctataagtgatagaatgaaaggtgactatatctggcaagtgggtgatgtgcgtttattctatacaaatggttctgtgcatggcgaaattgtagaagttaacgatggtagtaaaacagctctatttcgttttgaaaacacagcagatagaaacccaaatcaaattgttattgtcccagaaagagataatcgtcatggattatctcctcgaggagatcgcatgataatggatatgcattttattaatggtagtgatggagaaaaatacaaatatgtagttggtcacggtaattcagatcgatactacggtacactatttgcaaccaaaaaagataaagaa

SEQ ID NO: 85 - protein, 326 aa, Pasteurella multocida
CSGGGGGNNNVPHPPVEKRTVATTQQVAAKPTPPSESLVKRVLNNSENNPPSRESEKKASQTPPQADSAKSTIQSTPAVIPDSTNSLTSKKGQIQMSPEWIGKEEKKYAQYSWEHTPESIPVFKLIENNQYKYVNDKYFTLEPINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERDNRHGLSPRGDRMIMDMHFINGSDGEKYKYVVGHGNSDRYYGTLFATKKDKE

SEQ ID NO: 86 - DNA, 978 bp, Pasteurella multocida
  source 1..978: mol_type = genomic DNA
tgtagtggcggtgggggcggtaataaaaaagtaccacaccctcctgtagaaaaacgtactgtggcgacaacacaacaagttgctgcaaaaccaactccgccttctgaaagtctagttaaacgagtactcaataattcagagaataacccaccatcaagagagtcagagaaaaaagcctcacaaactccaccacaaacagatagcgcgaaatcaacaatacaatctactcccgctgttataccagatagtacaaactctttaacatctaagaaaggacaaattcaaatgtctccagaatggattgggaaagaagaaaaaaagtatgcgcaatattcatgggaacatacgccagaatctattccagtatttaaattaattgaaaataaccaatacaaatatgttaatgataagtactttacacttgaatcaatcaatcttaatttgactaaagaaaatcaagttaaagaaggttcttatcaatttagtctattagactctggagtctattatggccattacttaagctcaaacgatggtattcatccagaatataactttgtgattgcttttgacaaaaacagagaatacacactgaaagatatcacagctgaatactataattcggagggtttcaattatgctataagtgatagaatgaaaggtgactatatctggcaagtgggtgatgtgcgtttattctatacaaatggttctgtgcatggcgaaattgtagaagttaacgatggtagtaaaacagctctatttcgttttgaaaacacagcagatagaaacccaaatcaaattgttattgtcccagaaagaaataatcgtcatggattatctcctcgaggagatcgcatgataatggatatgcattttattaatggtagtgatggagaaaaatacaaatatgtagttggtcacggtaattcagatcgatactacggtacactatttgcaaccaaaaaagataaagaa

SEQ ID NO: 87 - protein, 326 aa, Pasteurella multocida
CSGGGGGNKKVPHPPVEKRTVATTQQVAAKPTPPSESLVKRVLNNSENNPPSRESEKKASQTPPQTDSAKSTIQSTPAVIPDSTNSLTSKKGQIQMSPEWIGKEEKKYAQYSWEHTPESIPVFKLIENNQYKYVNDKYFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIVEVNDGSKTALFRFENTADRNPNQIVIVPERNNRHGLSPRGDRMIMDMHFINGSDGEKYKYVVGHGNSDRYYGTLFATKKDKE

SEQ ID NO: 88 - DNA, 978 bp, Pasteurella multocida
  source 1..978: mol_type = genomic DNA
tgtagtggcggtgggggcggtaataacaatgtaccacaccctcctgtagaaaaacgtactgtggcgacaacacaacaagttgctgcaaaaccaactccgccttctgaaagtctagttaaacgagtactcaataattcagagaataacccaccatcaagagagtcagagaaaaaagcctcacaaactccaccacaaacagatagcgcgaaatcaacaatacaatctactcccgctgttataccagatcgtacaaactctttaacatctaagaaaggacaaattcaaatgtctccagaatggattgggaaagaagaaaaaaagtatgcgcaatattcatgggaacatacgccagaatctattccagtatttaaattaattgaaaataaccaatacaaatatgttgatgataagtgctttacacttgaatcaatcaatcttaatttgactaaagaaaatcaagttaaagaaggttcttatcaatttagtctattagactctggagtctattatggccattacttaagctcaaacgatggtattcatccagaatataactttgtgattgcttttgacaaaaacagagaatacacactgaaagatatcacagctgaatactataattcggagggtttcaattatgctataagtgatagaatgaaaggtgactatatctggcaagtgggtgatgtgcgtttattctatacaaatggttctgtgcatggcgaaattatagaagttaacgatggtagtaaaacagctctatttcgttttgaaaacacagcagatagaaacccaaatcaaattgttattgtcccagaaagagataatcgtcatggattatctcctcgaggagatcgcatgataatggatatgcattttattaatggtagtgatggagaaaaatacaaatatgtagttggtcacggtaattcagatcgatactacggtacactatttgcaaccaaaaaagataaagaa

SEQ ID NO: 89 - protein, 326 aa, Pasteurella multocida
CSGGGGGNNNVPHPPVEKRTVATTQQVAAKPTPPSESLVKRVLNNSENNPPSRESEKKASQTPPQTDSAKSTIQSTPAVIPDRTNSLTSKKGQIQMSPEWIGKEEKKYAQYSWEHTPESIPVFKLIENNQYKYVDDKCFTLESINLNLTKENQVKEGSYQFSLLDSGVYYGHYLSSNDGIHPEYNFVIAFDKNREYTLKDITAEYYNSEGFNYAISDRMKGDYIWQVGDVRLFYTNGSVHGEIIEVNDGSKTALFRFENTADRNPNQIVIVPERDNRHGLSPRGDRMIMDMHFINGSDGEKYKYVVGHGNSDRYYGTLFATKKDKE

SEQ ID NO: 90 - DNA, 879 bp, Pasteurella multocida
  source 1..879: mol_type = genomic DNA
tgtagtggtggtgggggtggtaataatccatcacaatatcctaaagaagtacgccctgtggtaactgaacaagccaaaacagctaaagaagtgattttacctacaccctctattggatcagaacagtcatctcaaaacgtaatagctaaagatgctttcaatgtgagcgaaaaaacgcatcaacactccaaaccagaaaaaattgcaaaagatattaattggacaggtagtgctgtgtcgtctataggctccacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaacctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccag

SEQ ID NO: 91 - protein, 293 aa, Pasteurella multocida
CSGGGGGNNPSQYPKEVRPVVTEQAKTAKEVILPTPSIGSEQSSQNVIAKDAFNVSEKTHQHSKPEKIAKDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLTYREGKVSGEIFGQNNVKHFSFMNSNKIDPNSIVIVPEEAHRYLSRNDKMFLEMHFINGENGEKYKYIVGSGKTDKYYGALFATKQENQ

SEQ ID NO: 92 - DNA, 680 bp, Pasteurella multocida
  source 1..680: mol_type = genomic DNA
atgcaccatcatcatcaccatcaccatggatcccttataataaagatgctgtatttacatacgaattaatcgcaaatccagatgcagattatagcgatcaaaaattgattcttaaaaaagaaattagttatatcaaacttaatctaggcataaatcaagacaataaaaatgcaccaagctatatttttaatttactagatgacaatgtctattatggtttttatcgtgatactcaagatatgaacagaatagaaaataaatatacttatgctttcaaaaaagaagctgaaaattttgataaccttcaaaaatttaatgcgacttatgaaggtcaattttggttctctagtatagatactccaaatgtacctactgttgcaagagcattcttaacatataacaatggtagagtggatggtgaaatattggctaaacattggaatgaaaaattgtttcagattactggatttgataataatcctcgtaaagtagaaatttttcctactgtagaatatttacctaattcgggaacaagactaacaaaaggagccacatcacctcatcgtttccaaatggacttacatttcatcaatagtacaaatggtgaaaaaaacaaatatcttgtcggccaaggtagtactgagcagtactggggtgttttaggtatggagaaaaaacaa

SEQ ID NO: 93 - protein, 227 aa, Pasteurella multocida
MHHHHHHHHGSSYNKDAVFTYELIANPDADYSDQKLILKKEISYIKLNLGINQDNKNAPSYIFNLLDDNVYYGFYRDTQDMNRIENKYTYAFKKEAENFDNLQKFNATYEGQFWFSSIDTPNVPTVARAFLTYNNGRVDGEILAKHWNEKLFQITGFDNNPRKVEIFPTVEYLPNSGTRLTKGATSPHRFQMDLHFINSTNGEKNKYLVGQGSTEQYWGVLGMEKKQ

SEQ ID NO: 94 - DNA, 702 bp, Pasteurella multocida
  source 1..702: mol_type = genomic DNA
atgcaccatcatcatcaccatcaccatggatccgatattaattggacaggtagtgctgtgtcgtctataggctctacttggtggcaacataaaccaaataatattcctgtttttacactaattcttaatgaaaaccaaatttactccgatgaaaaaaacttcactctagaacaaaaacatattgatcttacaaagaataatattttggatgaaaagtactttcatttcacattattagactccggaatttattatggaaattatttaagttcatacgatcagatgaacgcagaatctaattatgtttttgcatttgataaagatcgtgaatatacaggaaaagatatcaccgcaaattattatggtaatgaaggatttaaatattctgtaaaattaggtaatacatacctatcacaggtgggtgatgttcacttaatctatcgagaagggaaagtatcaggagaaatttttggtcaaaataacgtaaaacactttagctttatgaatagcaataaaattgacccaaatagcattgttattgtcccagaagaagcacatcgatatctatccagaaatgataaaatgttcttagaaatgcatttcattaatggggaaaatggagaaaaatataaatatattgtgggtagtggtaaaacggacaaatactacggcgcattatttgcaactaaacaagaaaaccag

SEQ ID NO: 95 - protein, 234 aa, Pasteurella multocida
MHHHHHHHHGSDINWTGSAVSSIGSTWWQHKPNNIPVFTLILNENQIYSDEKNFTLEQKHIDLTKNNILDEKYFHFTLLDSGIYYGNYLSSYDQMNAESNYVFAFDKDREYTGKDITANYYGNEGFKYSVKLGNTYLSQVGDVHLIYREGKVSGEIFGQNNVKHFSFMNSNKIDPNSIVIVPEEAHRYLSRNDKMFLEMHFINGENGEKYKYIVGSGKTDKYYGALFATKQENQ

SEQ ID NO: 96 - DNA, 801 bp, Pasteurella multocida
  source 1..801: mol_type = genomic DNA
atgcaccatcatcatcaccatcaccatggatcctctagtttctattcagcaaaagcgccatcacaagtatctcacgataaaagatcggtacactggaaaggagaatcagtttctgaaaaagaacatcttgacttctcttacagtaaagatgccgtatttacccgtcatttggtaacaaatccaaacgctgtttatagcactgatcccaacctgattagtaaagacattaaatatattacattaaccactactggatataatcaagataataagagcggtccaaactatgagttaaatttgctcgatgaaaatatttattacggttactatcgtgattcacaagatatgaatcatgttgaaaatatctatgtatatgggttcaaaaaagatgcagaaaatcaagataatcttcaatttctaacagcaaattaccaaggagaatttttattctcaacggcaacaaacccaaatgtgccggtactaggtaaagctgtcttaaattacaaagaaggaaaagctaaaggcgaaattctagaacgagatagcaattacaaattatttgatatttatgttaatgagcgccctaatcaagcaattttgaatcctgtagctgaaagactacctacctctgatcttattatgaatacacgtaaaaattcaccagaccgtgtcacaatagatttacactttattaagggtcaagataaccaagaaaataaatatattgtaggtcaagggggtaatgaaaaatattggggcgtattaggtttagagaaaaaagagaccaaggcagaaaaatag

SEQ ID NO: 97 - protein, 266 aa, Pasteurella multocida
MHHHHHHHHGSSSFYSAKAPSQVSHDKRSVHWKGESVSEKEHLDFSYSKDAVFTRHLVTNPNAVYSTDPNLISKDIKYITLTTTGYNQDNKSGPNYELNLLDENIYYGYYRDSQDMNHVENIYVYGFKKDAENQDNLQFLTANYQGEFLFSTATNPNVPVLGKAVLNYKEGKAKGEILERDSNYKLFDIYVNERPNQAILNPVAERLPTSDLIMNTRKNSPDRVTIDLHFIKGQDNQENKYIVGQGGNEKYWGVLGLEKKETKAEK